(19) United States
(12) Patent Application Publication
(10) Pub. No.: US 2023/0390027 A1
(43) Pub. Date: Dec. 7, 2023

(54) AUTO-SMILE DESIGN SETUP SYSTEMS

(71) Applicant: VOYAGER DENTAL, INC., EDEN PRAIRIE, MN (US)

(72) Inventors: Michael C. Marshall, Edina, MN (US); John Madden, Minneapolis, MN (US); Stephen B. Siegfried Floe, Chanhassen, MN (US)

(21) Appl. No.: 18/328,625

(22) Filed: Jun. 2, 2023

Related U.S. Application Data
(60) Provisional application No. 63/348,217, filed on Jun. 2, 2022.

(51) Int. Cl. A61C 7/00; A61C 13/34; G06T 19/20; G06T 3/00; G16H 20/30

(52) U.S. Cl. CPC A61C 7/002; A61C 13/34; G06T 19/20; G06T 3/0081; G16H 20/30; A61C 2007/004; G06T 2219/2004; G16H 20/40

(57) ABSTRACT

Disclosed are systems and methods for performing an auto-setup of a smile design for a patient using a digital orthodontics model. A method can include accessing, by a computer system, a digital orthodontics model for a patient and an image of the patient showing the patient's existing teeth, accessing orthodontics rules with permissible degrees of movement for each tooth, determining, based on the rules, tooth movements along an arch form defined in the model to achieve a desired teeth arrangement for the patient, selecting a set of veneers from amongst sets of veneers to achieve the desired teeth arrangement based on the tooth movements, overlaying the digital orthodontics model with teeth of the selected set, augmenting the image of the patient's teeth with the model having the teeth of the selected set, and returning at least the augmented image.

### INCORPORATION BY REFERENCE

This application claims priority to U.S. Provisional Application Ser. No. 63/348,217, entitled “Auto-Denture Design System for Denture Replacement,” filed on Jun. 2, 2022, the disclosure of which is incorporated by reference in its entirety.

### TECHNICAL FIELD

This document generally describes computer-automated technology for designing dental appliances, such as veneers and/or caps, for a patient to improve, adjust, and/or enhance an appearance of the patient's smile.

### BACKGROUND

A patient's smile can be improved with smile design techniques. Smile designing includes creating a straight, white, and/or aesthetically-pleasing smile. Smile design can include cosmetic dental procedures. These procedures can correct imperfections in the patient's teeth, such as worn-down, misaligned, yellowed, and/or gapped teeth. Changing the patient's smile can be achieved with orthodontics, aligners, veneers, caps, or other types of dental appliances.

Veneers can be applied to the patient's teeth to fix stained, chipped, and/or gapped teeth. The veneers can improve an appearance of the patient's smile. The veneers can provide strength and resilience comparable to natural tooth enamel. Veneers can include custom-made shells of tooth-colored materials that can be designed to cover a front surface of the patient's teeth. The veneers can be customized to a shape of the patient's teeth and bonded to the patients' teeth original enamel during in-office procedures. The veneers can be made from porcelain and/or from resin composite materials.

Caps and crowns can be applied to the patient's teeth to improve a shape, size, and/or appearance of the patient's teeth and/or smile. These types of dental appliances can be made of porcelain or other similar materials. These types of dental appliances can encase an entire tooth surface and/or a portion of the tooth surface while also protecting and strengthening the tooth structure. Caps and crowns can be sized to fit measurements of the patient's teeth.

Various types of orthodontics treatment plans can be used to improve the patient's smile. Orthodontics can, for example, align the patient's teeth to resolve for overbite, straighten teeth, remove or fix gaps, or otherwise improve the patient's overall smile and appearance. An orthodontics treatment plan can include veneers, caps, crowns, braces, aligners, retainers, and other similar types of dental appliances. Braces, for example, can be designed to fit the patient's mouth and teeth and shift the teeth using brackets, bands, and/or wires. The braces can help resolve rotated teeth and/or overlapping of teeth. Aligners, as another example, can be a clear material that fits over the teeth and shifts the teeth using a series of custom trays to receive the teeth. The aligners can help resolve crooked and/or crowded teeth. Retainers, as yet another example, can be designed to keep the patient's teeth in a particular position/orientation and therefore prevent the teeth from drifting. Whereas veneers, caps, crowns, and/or braces may be attached to the patient's teeth to affect movement and/or change of appearance, aligners and retainers can be worn by the patient and removed when the patient desires (e.g., the aligners or retainers can be worn during the night while the patient is sleeping and removed during the day).

### SUMMARY

This disclosure generally describes systems, methods, and computer-automated techniques for generating a digital orthodontics model for improving a patient's smile and/or overall appearance of their teeth. The disclosed technology can provide techniques to design and iteratively and automatically adjust teeth in the model within permissible degrees of movement per tooth as defined by orthodontics rules to design veneers and/or orthodontics treatment plans for the patient. The disclosed technology can implement one or more machine learning models and/or artificial intelligence (AI) algorithms to perform operations including but not limited to generating a digital orthodontics model for the patient based on patient scan data and/or image data, defining an arch form using the model, and determining tooth movements along the arch form to achieve a desired teeth arrangement that also complies with permissible degrees of movement per tooth. The disclosed technology can perform operations including selecting veneers based on the tooth movements, overlaying the model with the selected veneers, iteratively adjusting the teeth in the model with the selected veneers, and augmenting the image of the patient with the model having the selected veneers and adjusted teeth. The disclosed technology can also perform operations including determining and returning the augmented image, tooth movement data, and/or a dental orthodontics treatment plan based on the model having the selected veneers and adjusted teeth. The disclosed technology can therefore be used to design an improved smile for the patient with computer-automated and efficient techniques.

The disclosed techniques can be used to improve a shape of the patient's smile and arrangement of their teeth (which can be based on the patient's facial features, positioning of the patient's eyes and eye sockets, a curve of spe, and/or a curve of Wilson, among other factors). For example, an aesthetic appearance of the patient's smile can be achieved by aligning an occlusal plane for the patient's teeth to be parallel with an angle and/or plane of the patient's pupils. Depending on a shape of the patient's mouth and/or lips, the disclosed technology can also provide automated teeth setups to enhance the patient's smile.

Traditionally, a dentist may take pictures of the patient's smile, then overlay the picture with a veneer design to determine what veneers to use for the patient and to show what the patient's new smile may look like. The disclosed technology, on the other hand, provides an automated process for efficiently and accurately determining a best veneer design for the patient using scan data of the patient's teeth. The disclosed technology can automatically determine a preferred set of veneers for the patient and preferred movement of the patient's teeth using the scan data to achieve a desired smile for the patient. Once the veneer design is determined, the disclosed technology can use image-processing techniques to augment an image of the patient with the determined veneer design. The augmented image can provide a realistic depiction of the patient with the veneer design that was uniquely determined for the patient.

Moreover, the disclosed technology accounts for movement of various teeth, not just front teeth, to determine the best veneer design for the particular patient (or orthodontics treatment plan, more generally). Traditionally, dentists may focus on moving the front teeth and not posterior teeth, such as molars. The molars may simply be grinded down because the molars are less visible and therefore less likely to impact the overall appearance of the patient's smile. However, ground-down molars can impact the patient's comfortability, especially making it challenging for the patient to chew food. The disclosed technology, on the other hand, can provide for iteratively adjusting positioning of all the patient's teeth in order to achieve a desired smile and overall teeth arrangement for the patient, without compromising the patient's comfortability and overall appearance.

Digital models of orthodontics can be used to create orthodontic dental appliances for patients. Designing these appliances can be time consuming for a dentist or technician. Each appliance may require a custom design to ensure patient fit and satisfaction. Conventional methods for producing these custom designs can be time-consuming and imprecise, and the results can be technician-dependent since much of the design process may rely on the technician making selections and design choices based on visual inspection of existing images or scans of the patient's teeth. The resulting dental appliances and/or orthodontics treatment plans may require grinding and other types of adjustments, and can be an imperfect fit, thereby impacting the patient experience. Rework and required readjustments to correct the fit of the designed appliances can drive cost and inefficiency for patients, dentists, dental offices, and laboratories.

The disclosed technology can also provide for automatic detection of landmarks and characteristics in a digital orthodontics model produced from a scan of the patient's teeth and automated design of veneers or other similar dental appliances based on the model and detected landmarks and characteristics. The automatic detection of landmarks of teeth in a digital tooth scan can enable efficient and accurate selection of a set of veneers, and placement of the selected veneers in the digital model for use in preparing an orthodontics treatment plan for the patient. The automatic selection can be more efficient than selection by a technician based on visual inspection and can also reduce variability in veneers designed by different technicians. The resulting denture design can be used to design veneers and their placement that is a closer match to a desired appearance for the patient in fit, function, and aesthetics, thereby improving the patient experience and acceptance of the veneers.

For example, the disclosed technology can automatically select an occlusal plane, an arch size and shape, and best set of veneers from a library based on a CT or cone beam computed tomography (CBCT) scan of the patient's teeth. After selection of the veneers, software or other computer systems described herein can determine a position of each tooth in a digital orthodontics model using a closest fit algorithm, such as iterative closest point (ICP). The selected veneers can additionally be down-sampled prior to fitting to the model to enhance efficient processing of the model.

The teeth can be auto-populated in the model by a computer system, and can be displayed in a GUI for further manipulation and/or adjustment by a technician. Using heat maps and/or color maps, fit of the teeth in the model can be efficiently conveyed to the technician to aid in review of the model and further optional adjustments for the placement and design of the veneers. During adjustments of teeth positioning, the GUI can present the teeth in the display and allow for movement of a tooth or a group of teeth relative to one another while avoiding interference with other teeth through overlap or collision. These adjustments can also be made within permissible degrees of movement per tooth type, which can be defined in one or more existing orthodontics rules. In some implementations, the technician follows a process flow in the GUI to accept or adjust the teeth in the model using the ICP algorithms, thereby efficiently completing the design process for the patient's veneers and/or overall orthodontics treatment plan. Accordingly, the technician can accept computer-automated processes to perform iterative operations in adjusting the teeth in the model until a desired veneers design is achieved.

One or more embodiments described herein include a method for performing an auto-setup of a smile design for a patient using a digital orthodontics model, the method including: accessing, by a computer system, (i) a digital orthodontics model for a patient comprising upper teeth and lower teeth and (ii) an image of the patient, the image including at least the patient's existing teeth, accessing, by the computer system and from a data store, orthodontics rules comprising permissible degrees of movement for each tooth, determining, by the computer system and based on the orthodontics rules, tooth movements along an arch form defined in the digital orthodontics model to achieve a desired teeth arrangement for the patient, the tooth movements being made within the permissible degrees of movement for each tooth, selecting, by the computer system and from the data store, a set of veneers from amongst a group of sets of veneers to achieve the desired teeth arrangement for the patient, the set of veneers being selected based at least in part on the determined tooth movements, overlaying, by the computer system, the digital orthodontics model with teeth of the selected set of veneers, augmenting, by the computer system, the image of the patient's existing teeth with the digital orthodontics model having the teeth of the selected set of veneers, the augmented image including a realistic visual depiction of the patient with the teeth of the selected set of veneers, and returning, by the computer system, at least the augmented image.

The embodiments described herein can optionally include one or more of the following features. For example, the method can include generating, by the computer system, tooth movement instructions based on the determined tooth movements and the digital orthodontics model having the teeth of the selected set of veneers. The method can include generating, by the computer system, an orthodontics treatment plan based on the determined tooth movements and the digital orthodontics model having the teeth of the selected set of veneers. The method can include generating and returning veneers information based on the digital orthodontics model having the teeth of the selected set of veneers, the veneers information including a type of the selected set of veneers and placement of the selected set of veneers in the patient's mouth.

As another example, the method can include iteratively adjusting, by the computer system, positions of one or more teeth in the digital orthodontics model based on the orthodontics rules and until the desired teeth arrangement for the patient can be achieved by the iterative adjustments. In some implementations, determining, by the computer system and based on the orthodontics rules, tooth movements along an arch form defined in the digital orthodontics model to achieve a desired teeth arrangement for the patient can include: leveling each tooth relative to an occlusal plane within respective permissible degrees of leveling movement defined by the orthodontics rules for the tooth. Sometimes, determining, by the computer system and based on the orthodontics rules, tooth movements along an arch form defined in the digital orthodontics model to achieve a desired teeth arrangement for the patient can include: rotating at least one tooth around a reference line perpendicular through marginal ridges of the at least one tooth, the rotating being within permissible degrees of rotating movement defined by the orthodontics rules for the at least one tooth.

Sometimes, determining, by the computer system and based on the orthodontics rules, tooth movements along an arch form defined in the digital orthodontics model to achieve a desired teeth arrangement for the patient can include: torqueing at least one tooth using at least one of buccal cusp tips and distal cusp tips of the at least one tooth, the torqueing being within permissible degrees of torqueing movement defined by the orthodontics rules for the at least one tooth. As another example, determining, by the computer system and based on the orthodontics rules, tooth movements along an arch form defined in the digital orthodontics model to achieve a desired teeth arrangement for the patient can include: tipping at least one tooth at least one of mesially and distally using marginal ridges of the at least one tooth, the tipping being within permissible degrees of tipping movement defined by the orthodontics rules for the at least one tooth. As another example, determining, by the computer system and based on the orthodontics rules, tooth movements along an arch form defined in the digital orthodontics model to achieve a desired teeth arrangement for the patient can include: snapping at least one tooth to the arch form, the snapping being within permissible degrees of snapping movement defined by the orthodontics rules for the at least one tooth.

In some implementations, determining, by the computer system and based on the orthodontics rules, tooth movements along an arch form defined in the digital orthodontics model to achieve a desired teeth arrangement for the patient can include: adjusting interproximal (IP) contacts between adjacent teeth, the adjusting being within permissible degrees of IP-contact movement defined by the orthodontics rules for the at least one tooth. Adjusting IP contacts can include: for each tooth of the adjacent teeth, generating a bounding box, identifying a center point of the tooth as a center point in the bounding box, identifying a vector between center points of the adjacent teeth, a length of the vector being equal to or within respective permissible degrees of IP-contact movement for the adjacent teeth, and moving at least one tooth of the adjacent teeth along the vector and within the respective permissible degrees of IP-contact movement for the adjacent teeth to maintain relative orientation, remove overlap, and put the adjacent teeth in contact at a predefined contact point.

Sometimes, selecting, by the computer system and from the data store, a set of veneers from amongst a group of sets of veneers to achieve the desired teeth arrangement for the patient can include: identifying a shape of the patient's existing teeth in the digital orthodontics model, scoring each set of the group of sets of veneers based on at least one of (i) a likelihood that the set achieves the desired teeth arrangement and (ii) the set has a shape similar to the shape of the patient's existing teeth, and selecting the set of veneers from the group of sets of veneers having a highest score. Selecting the set of veneers can include selecting a subset of the group of sets of veneers, wherein each set of veneers in the subset has a score that exceeds a threshold score value.

As another example, determining, by the computer system and based on the orthodontics rules, tooth movements along an arch form defined in the digital orthodontics model to achieve a desired teeth arrangement for the patient can include, for each tooth: determining a starting point of the tooth, identifying, based on the orthodontics rules, a maximum permissible degree of movement for the tooth, determining an offset between the starting point of the tooth and the maximum permissible degree of movement for the tooth, and moving the tooth according to the determined offset.

The method can also include transmitting the augmented image to a user device for presentation in a graphical user interface (GUI) at the user device. The method can include transmitting, by the computer system, the digital orthodontics model with the teeth of the selected set of veneers to a user device for presentation in a GUI at the user device, receiving, by the computer system and from the user device, user input indicating approval of the selected set of veneers, and generating, by the computer system and based on receiving the user input, an orthodontics treatment plan for (i) adjusting the patient's existing teeth according to the determined tooth movements and (ii) applying the teeth of the selected set of veneers to the patient's existing teeth. Sometimes, the method can include receiving, by the computer system and from the user device, user input indicating a request for other sets of veneers from the group of sets of veneers, wherein the other sets of veneers do not include the selected set of veneers, selecting, by the computer system and from amongst the group of sets of veneers, the other sets of veneers, and transmitting, by the computer system to the user device, the other sets of veneers for presentation in the GUI at the user device, the presented other sets of veneers being user-selectable.

One or more embodiments described herein include a method for performing an auto-setup of a smile design for a patient using a digital orthodontics model, the method including: accessing, by a computer system, patient data including (i) a digital orthodontics model for a patient, the digital orthodontics model including upper teeth and lower teeth and (ii) an image of the patient, the image including at least the patient's existing teeth, selecting, by the computer system and from a data store, a set of veneers from amongst a group of sets of veneers to achieve a desired teeth arrangement for the patient, the set of veneers being selected based at least in part on the patient data, overlaying, by the computer system, the digital orthodontics model with teeth of the selected set of veneers, retrieving, by the computer system and from the data store, orthodontics rules having permissible degrees of movement for each tooth, determining, by the computer system and based on the orthodontics rules, tooth movements along an arch form defined in the digital orthodontics model to achieve the desired teeth arrangement for the patient of the digital orthodontics model with the teeth of the selected set of veneers, the tooth movements being made within the permissible degrees of movement for each tooth, for each tooth movement, determining, by the computer system, whether the tooth movement satisfies corresponding permissible degrees of movement for the tooth, generating, by the computer system and based on a determination that the tooth movement satisfies the corresponding permissible degrees of movement for the tooth, tooth movement data, augmenting, by the computer system, the image of the patient's existing teeth with the digital orthodontics model having the teeth of the selected set of veneers and the determined tooth movements, the augmented image including a realistic visual depiction of the patient with the teeth of the selected set of veneers, and returning, by the computer system, at least the tooth movement data and the augmented image.

The method can optionally include one or more of the abovementioned features. The method can also optionally include one or more other features. For example, the method can also include generating, by the computer system and based on the tooth movement data, an orthodontics treatment plan, and returning, by the computer system, the orthodontics treatment plan for adjusting the patient's existing teeth according to the determined tooth movements.

Certain implementations may provide one or more advantages. For example, the disclosed technology can generate improved and better veneer designs over technician-based designs, and can do so in a manner that is computationally efficient (e.g., uses minimal computational resources, such as CPU cycles, RAM, network bandwidth). For example, a challenge that is posed with traditional veneer design is a multitude of potential variation in orientation and positioning of teeth in 3D space relative to each other and to opposing archways—resulting in a near infinite number of possible arrangements and setups for consideration. The disclosed technology can be used to iteratively prune a universe of possible veneer teeth arrangements to efficiently arrive at an appropriate veneer design for a patient in a manner that minimizes the computational resources required to perform such techniques

The disclosed technology also can provide for automating and efficiently combining multiple processes into a streamlined design process. Traditionally, a process for improving the patient's smile can include multiple sub-processes. Each of the sub-processes can be performed by different users. The design process can take a long time to complete, because the overall process can be dependent on completion of the multiple sub-processes. For example, an orthodontic may manually design desired teeth shape for the patient while a technician may receive the designed teeth shapes and further adjust their color, shape, position, etc. before an orthodontics treatment plan can be finalized and used on the patient. The disclosed technology, on the other hand, provides a single, automated design setup process for performing the sub-processes as part of one efficient process. As a result, veneers or other orthodontic treatment plans can be designed efficiently, quickly, accurately, in less time, and at lower costs than traditional processes.

Similarly, using software to automate portions of a smile design process can improve efficiency over technician development of smile designs based on visual identification of smile and teeth landmarks. A CT scanning device, CBCT scanning device, or other scanning device can quickly produce a scan of existing teeth that can be used by a computer program to identify a matching set of veneers (e.g., a veneers library), automatically determine various landmarks in a digital model produced from the scan, and position and align teeth in the model with the set of veneers to achieve a desired smile for the patient. The process for designing a smile can be significantly longer if a technician completes these steps based on visual inspection of the patient's existing teeth (such as in a picture of the patient's smile) or existing teeth scan and manual manipulation of an orthodontics model.

As another example, automating the smile design process can enable standardization of the process of producing a digital smile design. Landmarks and best-fits can be efficiently determined using the disclosed technology and iterative best fit algorithms can be applied to improve accuracy and efficiency in designing the patient's smile. The automatically produced smile designs can be of higher-quality than technician-produced designs, especially since the auto-designed veneers, teeth, or other orthodontic dental appliances can more closely align with the patient's teeth in scan data and/or a desired smile for the patient. The disclosed technology can also reduce variability in smile designs made by different technicians who generate designs by visual and manual inspection. In turn, automating the design process can result in smile designs that are accurate and thus used to treat the patient to achieve a desired fit, function, and appearance of their teeth.

In another example, automating the design process as described herein can significantly speed up the design process by suggesting libraries of veneers that are a close match to the teeth of a desired smile for the patient, and automatically adjusting positions and orientations of teeth in the smile design to achieve the desired fit and appearance of the veneers for the patient. Because the program can make suggestions and automatic adjustments of best-fit teeth positions and selections, the work that is required of a technician can be significantly reduced. The technician can simply review the suggestions and make minor adjustments, if needed, to the digital model rather than create an entire model from scratch. The amount of technician time required for creating a digital smile design can be decreased and throughput of smile designs can be increased. This automated process can also reduce costs and an amount of time required in the overall design process. Similarly, automating the techniques described herein allows for less-skilled technicians or other users to review and design dental appliances.

Similarly, the disclosed technology provides for more computationally efficient (e.g., use fewer CPU cycles, require less RAM for identifying appropriate library) and accurate selection of tooth libraries (e.g., veneers) for a particular patient, which can additionally be done without requiring a user's visual inspection, manual review, and manual manipulation of hundreds if not thousands of tooth libraries from which to select. For example, the disclosed technology can be used to identify an appropriate tooth library for a particular patient and their unique dimensions (e.g., dimensions of their teeth, gums, mouth, face, etc.) so that it fits the patient well and provides a pleasing visual aesthetic for the patient, while also performing these identifications in a manner that is computationally efficient yet still takes into consideration the patient's 3D geometry. Instead of using image analysis techniques that may identify tooth libraries that provide a good visual aesthetic for a patient but which may not account for the patient's full 3D geometry, the disclosed technology can provide both beneficial outcomes and in a manner that can avoid some of the more computationally resource intensive operations involved with image-based analysis. Additionally, the disclosed technology can provide better and more accurate tooth library selection over human-based selection, and in a manner that is more efficient and cost effective.

Moreover, the disclosed technology provides for adjusting all teeth relative to each other, not just front teeth, which may be done with traditional veneer design techniques described above. Here, the teeth maintain their relationship to one another during movement into occlusion in one dimension while they move and pivot in other dimensions. Fewer processing cycles may be used to automatically move a tooth into contact than would be used to generate a user interface and receive user inputs to position a single tooth in contact with another tooth, such as the front teeth visible when the patient smiles.

Another benefit of automatically moving all the teeth into contact is that a resulting arrangement of digital teeth may be more consistently of high quality than an arrangement where each digital tooth is moved into contact by a user and/or only some of the teeth are moved into contact, such as the front teeth visible when the patient smiles. In some implementations, multiple digital teeth may be selected and moved together to improve computing efficiency and accuracy in moving and aligning teeth to achieve a preferred smile design and model. Traditionally, a dentist may spend significant amounts of time visually analyzing patient tooth data with color or other visual maps to determine if neighboring and/or opposing teeth are touching or otherwise in preferred contacts. The disclosed technology, on the other hand, provides for automatically checking and resolving teeth contacts so that the teeth are appropriately aligned/in contact. These automated techniques can speed up the design process while maintaining accuracy and high quality arrangement of teeth for any patient.

Additional advantages will be apparent to the person of skill in the art based on the figures, description, and claims herein.

### DESCRIPTION OF THE DRAWINGS

FIG. 1A is a conceptual diagram of a system for computer-automated design of veneers for a patient in which veneers are selected for the patient based on determining permissible teeth movements for the patient.

FIG. 1B is a conceptual diagram of a system for computer-automated design of veneers for a patient based on determining whether permissible teeth movements can be made to achieve a look created by the veneers.

FIG. 1C is a block diagram of system components for computer-automated smile design and/or orthodontics treatment planning.

FIG. 2 is a flowchart of an example process for automatic smile design.

FIGS. 3A, 3B, and 3C are a flowchart of a process for computer-automated smile design for a patient.

FIGS. 4A and 4B are a flowchart of another process for computer-automated smile design for a patient.

FIG. 5 is a flowchart of a process for computer-automated selection of veneers for smile design without requiring movement of a patient's teeth.

FIG. 6 illustrates example adjustments of rotating, tipping, and/or torqueing one or more teeth in a digital orthodontics model.

FIG. 7A is an example GUI that may be generated by the digital smile design system for identifying an arch of a digital orthodontics model.

FIG. 7B illustrates snapping one or more lower teeth to an arch of a digital orthodontics model.

FIG. 7C illustrates an arch form for both upper and lower teeth of a digital orthodontics model.

FIG. 8 illustrates adjusting teeth in a digital orthodontics model within permissible degrees of movement to resolve IP contacts.

FIG. 9 is a conceptual diagram of system components for selecting veneers.

FIGS. 10A and 10B are a flowchart of a process for automatically leveling teeth in a digital orthodontics model for auto-smile design.

FIG. 11 is a flowchart of a process for automatically snapping teeth to an arch form of a digital orthodontics model for auto-smile design.

FIG. 12 is a flowchart of a process for automatically resolving IP contacts in a digital orthodontics model for auto-smile design.

FIG. 13 is a flowchart of a process for automatically socking teeth in a digital orthodontics model for auto-smile design.

FIGS. 14A and 14B illustrate example GUIs that may be generated during an automated process of positioning selected veneers in a digital orthodontics model and automatically aligning and leveling each tooth in the model according to an orthodontics treatment plan.

FIGS. 15A, 15B, and 15C illustrate example GUIs that may be generated during a process of automatically aligning and leveling each tooth in a digital orthodontics model for auto-smile design.

FIGS. 16A, 16B, and 16C illustrate schematic diagrams of an example digital orthodontics model and example teeth for auto-smile design.

FIG. 17 is an example architecture of a computing device, which can be used to implement aspects according to the present disclosure.

Like reference symbols in various drawings indicate like elements.

### DETAILED DESCRIPTION

The disclosed technology generally provides systems, methods, and techniques for automatically designing a smile for a patient based on digital models developed from scans of the patient's mouth and with iterative fitting algorithms, veneers, caps, crowns, other orthodontics dental appliances, and/or orthodontic treatment plans. The use of iterative fitting algorithms can improve accuracy and efficiency of a process for designing the patient's smile. The speed with which a computer system can identify one or more sets of veneers from a scan of the patient's mouth can be faster than a conventional approach of visual identification of veneers by a technician. The computer system can identify multiple landmarks of teeth in a digital model for the patient to determine permissible teeth movements for achieving a desired smile for the patient, selecting veneers for the patient, and/or performing other orthodontics treatment plans. As a result, the disclosed technology can provide for auto-designing veneers and/or a smile for the patient that more likely achieves fit, function, and appearance criterion for the patient. Technicians can make adjustments to the digital model more efficiently using the disclosed technology, which can allow for manipulation of groups of teeth together while also preventing interference between the teeth and/or other discrepancies in the smile design. The use of the technologies described herein can provide a more efficient process for producing digital smile designs for patients.

Different technicians may make design selections differently or based on different criteria, resulting in variation in smiles designed by different technicians. It can be difficult to distinguish dimensional differences between teeth of varying shapes based on visual inspection, and requiring a technician to make these determinations can create numerous challenges. The guesswork sometimes required of a technician in determining a set of veneers that can achieve the patient's desired smile cause differences in fit and function for the patient. The technician selection of veneers based on visual inspection of the patient's teeth may also introduce variability in the output design. The patient's resulting smile can look different when the selected veneers vary from the smile design, and can fail to meet the expectations of patients. The disclosed technology provides techniques to auto-design smiles using digital orthodontics models and iterative algorithms so that veneers and other orthodontic dental appliances can be efficiently and accurately designed according to a patient's requirements for improving their smile and/or overall appearance.

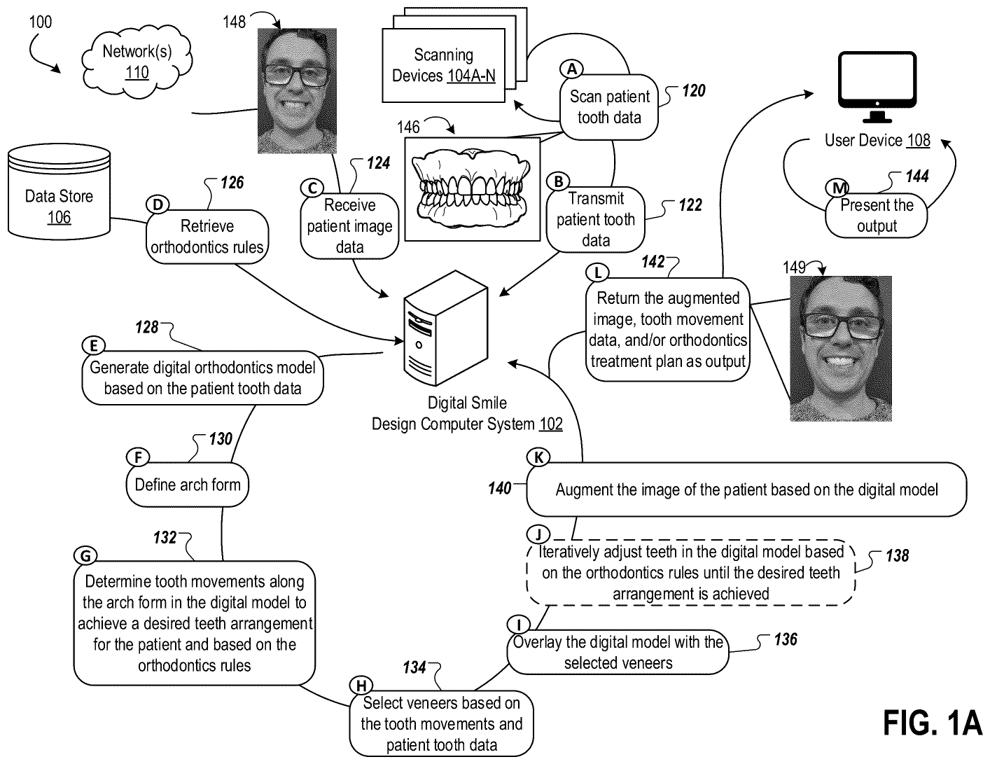

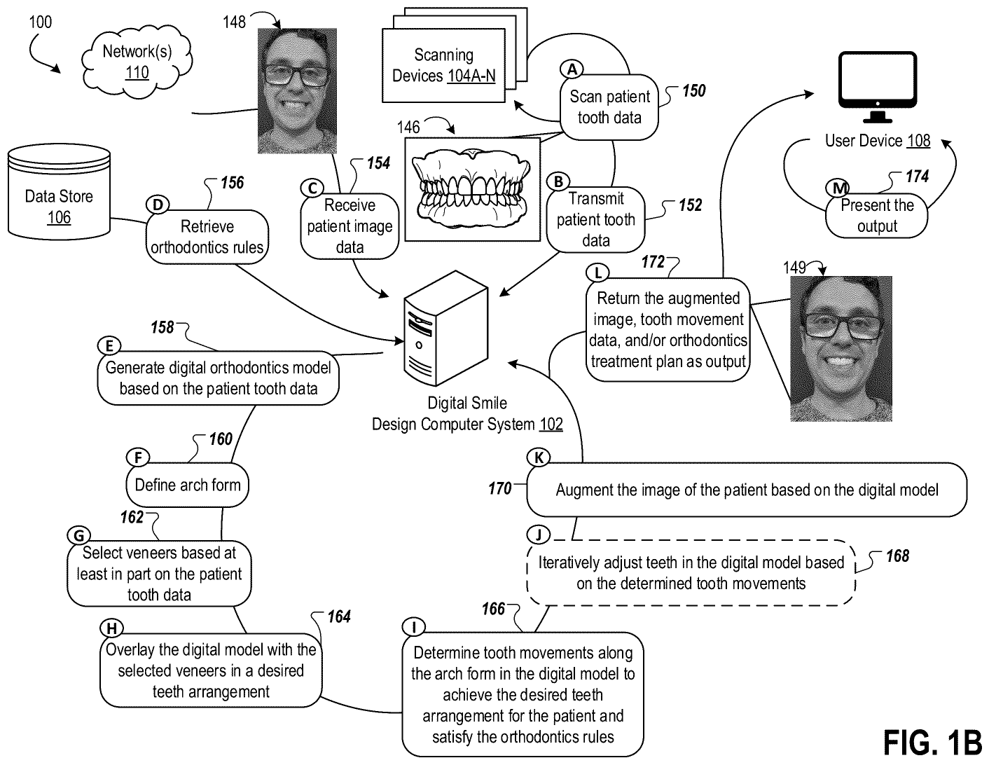

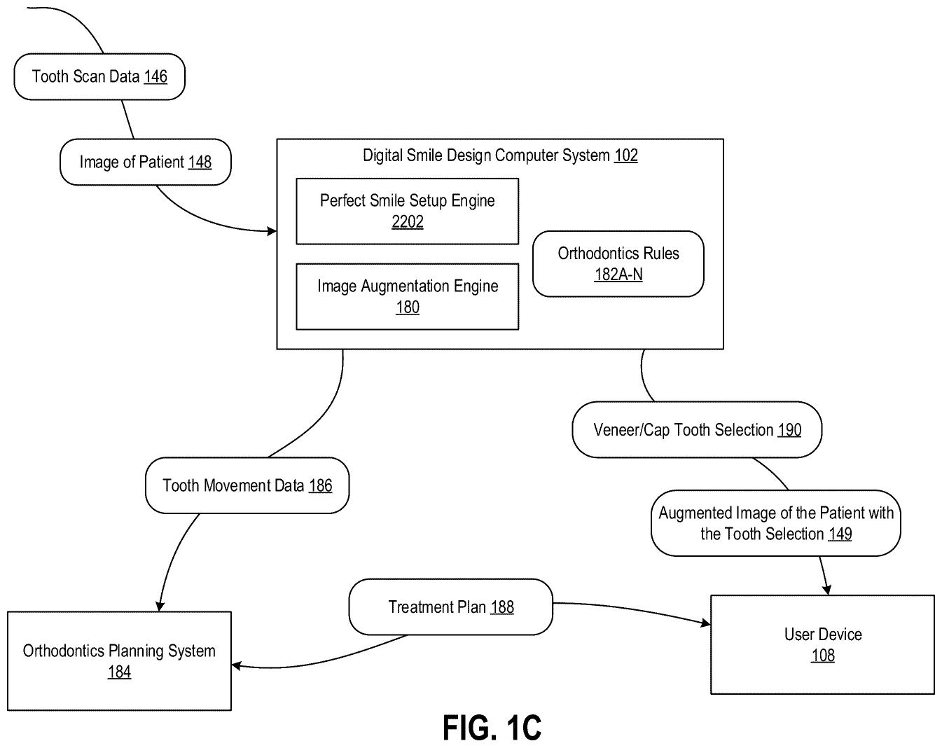

Referring now to the figures, FIG. 1A is a conceptual diagram of a system 100 for computer-automated design of veneers for a patient in which veneers are selected for the patient based on determining permissible teeth movements for the patient. Although FIG. 1A is described in reference to designing the patient's smile with selection of veneers, operations performed in FIG. 1A can also be performed to design the patient's smile with caps, crowns, aligners, retainers, braces, other types of orthodontic dental appliances, and/or other types of orthodontics treatment plans.

The system 100 can include a digital smile design computer system 102 (e.g., the ‘computer system’), scanning devices 104A-N, a data store 106, and/or a user device 108 that communicate (e.g., wired, wirelessly) with each other via network(s) 110.

The computer system 102 can be any type of computing system, cloud-based computing system, computing device, and/or network of computer systems. The computer system 102 can be remote from dental offices or other locations where a patient may go to get fitted for and receive dental appliances, such as veneers, caps, crowns, braces, aligners, retainers, etc. In some implementations, the computer system 102 can be in the dental office and/or part of the user device 108 or other computer system in the dental office. The computer system 102 can be configured to generate dental appliance designs unique to each patient based on processing patient-specific data, selecting tooth libraries, such as sets of veneers, that satisfy one or more selection criteria, generating a digital orthodontics model for the patient, and using the selected tooth libraries to design orthodontics dental appliances for the patient in the model with iterative algorithms.

The scanning devices 104A-N can include but are not limited to intraoral scanners, computed tomography (“CT”) scanning devices, cone beam computed tomography (“CBCT”) scanning devices, and/or other types of 3D imaging devices that may be used to capture images and other scan data of patients' mouths, dentition, jaws, and/or faces, such as patient scan data 146. The scan data 146 can be a 3D image or other type of representation of the patient's mouth and/or existing dentures. In some implementations, the scan data 146 can be previously captured using the scanning device(s) 104A-N (e.g., when the patient visits their dentist's office), then stored in the data store 106.

The data store 106 can be any type of database, data store, data repository, and/or cloud-based storage system. The data store 106 can store many different sets of veneers (e.g., tooth libraries) that have been predefined and/or previously generated, which can be used by the computer system 102 to auto-design smiles for patients. The sets of veneers can be tooth libraries that are generic and applicable to all patients. In some implementations, the sets of veneers can be generated for particular types of teeth, particular groups of teeth, particular purposes (e.g., type of smile, facial proportions, overbite, facial profile appearance, whether lips are pronounced and/or recessed), particular patient demographics (e.g., age, gender, dental condition), etc. The sets of veneers can each contain data or metadata, which further can be used with the disclosed techniques. For example, each set of veneers can include a predefined coordinate system. The coordinate system can then be used by the computer system 102 in determining measurements of a respective tooth and/or arranging and setting up the tooth relative to other teeth for the patient's digital smile design model. Each set of veneers can include additional or other information, including but not limited to tooth measurements, color data, shape data, texture data, etc.

As an illustrative example of the coordinate system, tooth coordinate systems can be used for snapping a respective tooth. Once datums are identified for a tooth, the computer system 102 can construct a coordinate system for the tooth based on the identified datums. The coordinate system can be stored in a respective tooth library in the data store 106. The coordinate system can then be used by the computer system 102 to appropriately align/snap the tooth to the arch form once the computer system 102 selects the tooth library having the coordinate system from the data store 106. The tooth coordinate systems can be predetermined by the computer system 102 and stored with respective tooth libraries in the data store 106, as described herein. When the tooth libraries are retrieved and used for designing the patient's dental appliances, such as dentures, the computer system 102 can load the teeth coordinate systems into the model and use the coordinate systems to appropriately rotate or otherwise adjust each tooth. The coordinate system is especially beneficial for rotating canines since the canines each only have 1 datum. The 1 datum may not provide enough information for determining how to rotate the canine to the arch form.

The data store 106 may also store one or more orthodontics rules. The orthodontics rules can provide ranges of movements (e.g., tip, torque, rotation) that can be performed for each type of tooth in a patient's mouth. The ranges can indicate a maximum degree of movement that can be performed for the particular type of tooth. When designing the patient's smile, various constraints can be applied to tip, torque, rotation, and other types of movement that may be applied to the patient's teeth. This is because the movements are limited by the actual teeth of the patient (whereas when designing dentures, there are greater degrees of freedom in terms of movement because the design is not constrained by positions of actual teeth of the patient). Therefore, the orthodontics rules can define the constraints for teeth movement on a tooth-by-tooth basis. The computer system 102 can utilize the orthodontics rules to determine best ways to position the patient's teeth to achieve a desired smile and given those movement constraints.

The data store 106 may store patient data, including but not limited to patient information (e.g., age, gender, teeth condition, dental appliance type, procedure, and/or history, and/or demographic information), 3D image data (e.g., captured by the scanning devices 104A-N, such as the scan data 146), 2D image data (e.g., converted from the 3D image data by the computer system 102 and/or image 148 captured by imaging devices such as a camera), teeth scans (e.g., as generated or captured by the scanning device(s) 104A-N or other teeth imaging devices), and/or teeth measurements (e.g., as provided by a relevant user and/or automatically determined by the computer system 102). Various other information described herein can also be stored in the data store 106, such as previously made digital orthodontics models and/or historic information about previous orthodontics treatment plans for the patients, any of which can be used by the computer system 102 to auto-design veneers or other orthodontics dental appliances for patients.

The user device 108 can be any type of computing device including but not limited to smartphones, tablets, laptops, computers, mobile phones, mobile devices, and/or wearable devices. The user device 108 can be used by a relevant user, such as a technician, dentist, and/or orthodontist. The user device 108 can be configured to present and output information in GUIs about auto design of veneers for the patient to the relevant user. The user device 108 can also be configured to receive user input indicating selection of sets of veneers and/or manual manipulation of the design of the veneers, as described further herein.

Still referring to the system 100 in FIG. 1, the scanning devices 104A-N can scan patient tooth data, such as the scan data 146 (block A, 120). The scanning can be performed at any time before the computer system 102 auto-designs the veneers for the patient. The scanning devices 104A-N can then transmit the patient tooth data to the computer system 102 in block B (122). In some implementations, the patient tooth data can be stored in the data store 106 and then the computer system 102 can retrieve the patient tooth data at a time at which the computer system 102 auto-designs veneers for a particular patient. In some implementations, the scan data 146 can be transmitted first to the user device 108 and reviewed by the technician before the user device 108 transmits the patient tooth data to the computer system 102 for further processing. Sometimes, the computer system 102 can request the scan data 146 or other patient tooth data once the technician provides user input at the user device 108 indicating a desire to design dentures for a particular patient. Other times, the computer system 102 can automatically receive the patient tooth data when the patient tooth data is generated (e.g., when scans or images are taken of the patient's teeth and/or mouth) and/or at predetermined times (e.g., every 15 minutes, every 30 minutes, every 1 hour, every 5 hours, every 12 hours, every 24 hours).

The computer system 102 can also receive patient image data 148 in block C (124). The image data 148 can be captured by the dentist, technician, and/or patient. The image data 148 can be captured by one or more of the scanning devices 104A-N, such as a camera, mobile phone, smartphone, tablet, laptop, etc. The image data 148 can also be captured by any other type of imaging sensor and/or device. Like the scan data 146, the image data 148 can be captured at any time, stored in the data store 106, and/or later retrieved/accessed by the computer system 102.

In block D, the computer system 102 can retrieve one or more orthodontics rules from the data store 106 (126). The rules can be retrieved, for example, for each type of tooth that may be moved or otherwise adjusted in order to achieve a desired smile design for the patient. The rules, as described herein, can indicate threshold ranges of permissible degrees of movement that can be applied to each tooth in the patient's mouth. These ranges therefore can provide constraints for how much the patient's teeth can be moved to achieve the desired smile design. The orthodontics rules may also indicate what types of movements are permissible for each type of tooth and/or group of teeth (e.g., tipping, torqueing, rotating). The orthodontics rules may indicate one or more constraints on interference, contact, and/or overbite that may be permissible between one or more adjacent teeth.

The computer system 102 can generate a digital orthodontics model for the patient based on the patient tooth data (block E, 128). The model can include the patient's teeth, which were scanned and presented in the tooth data. The model can also include, in some implementations, a set of digital teeth that can be manipulated/adjusted in order to achieve the desired smile design for the patient.

Using the model, the computer system 102 can define an arch form in block F (130). The arch form can be automatically generated by the computer system 102. In some implementations, as described herein, the relevant user, such as a technician, may adjust the arch form on the digital model. For example, a curved line can be visually displayed over lower (or upper) teeth of the model. One or more selectable points/spheres (e.g., nodes) can also be presented as part of the curved line over the lower teeth of the model. The user can select, click, and/or drag any of the selectable points to adjust a shape of the curved line. The shape of the curved line corresponds to the defined arch form. The user can, for example, drag one or more of the selectable points along the curved line to set a portion of the curved line to go through a buccal side of anterior teeth in the model and to go through buccal cusps of posterior teeth in the model. When the user adjusts one point on one side of the curved line, the computer system 102 can automatically adjust another point opposite the user-adjusted point on an opposite side of the curved line. Therefore, the teeth of the model can be adjusted to be symmetrical. In some implementations, if desired, the user can select an option for an asymmetrical curve and thus adjustments on the one side of the curved line may not be mirrored on the opposite side of the curved line.

The computer system 102 can define a lower arch form first. The lower arch form can be mirrored for an upper arch form for inner arch coordination. Once mirrored, the user can optionally adjust the upper arch form. For example, the user or the computer system 102 can move the upper arch form to be positioned over an inside of a midline of upper teeth. The user and/or the computer system 102 can also adjust the upper arch form such that the corresponding curved line goes down through center grooves of posterior teeth in the upper arch. In some implementations, the lower and upper arch forms can be defined separately and/or differently. In some implementations, the upper arch form can be defined first, then used to define the lower arch form.

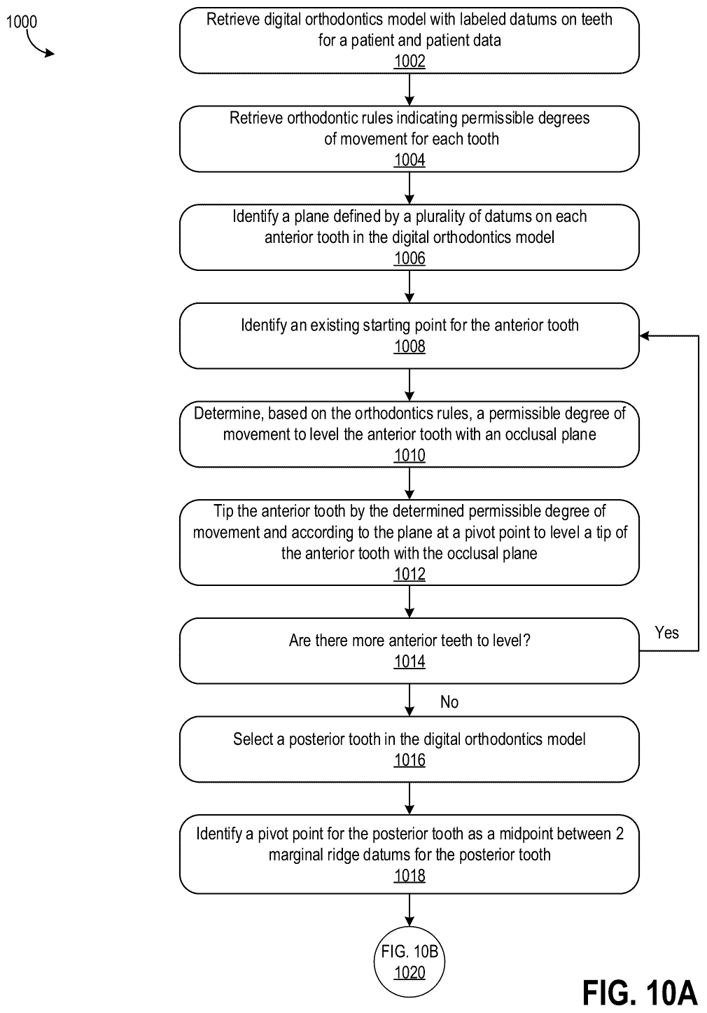

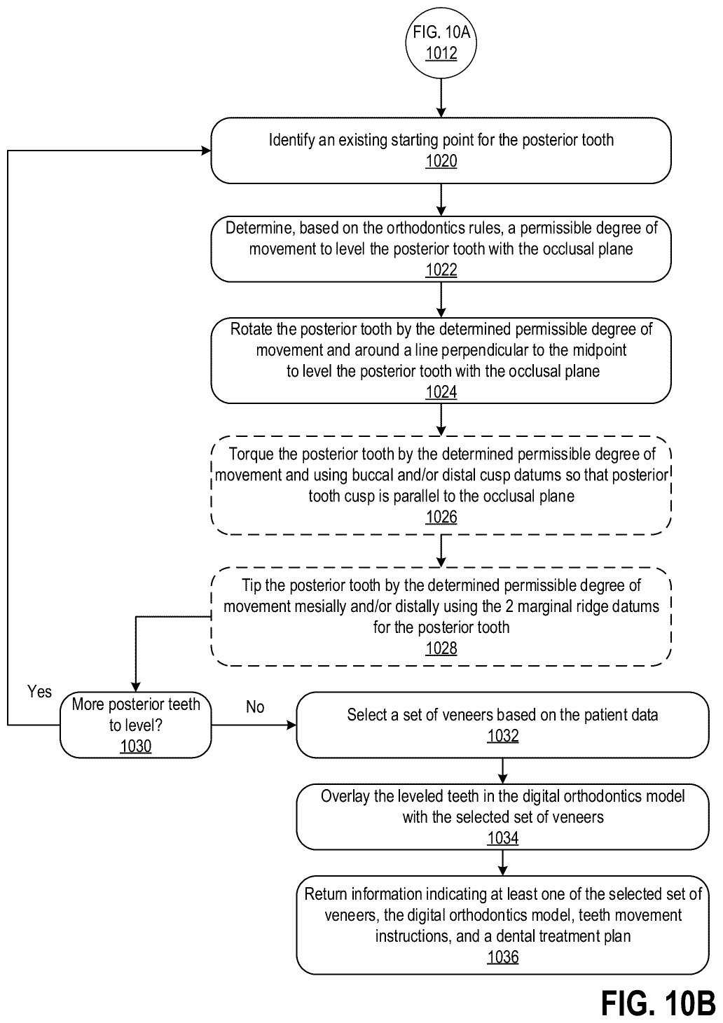

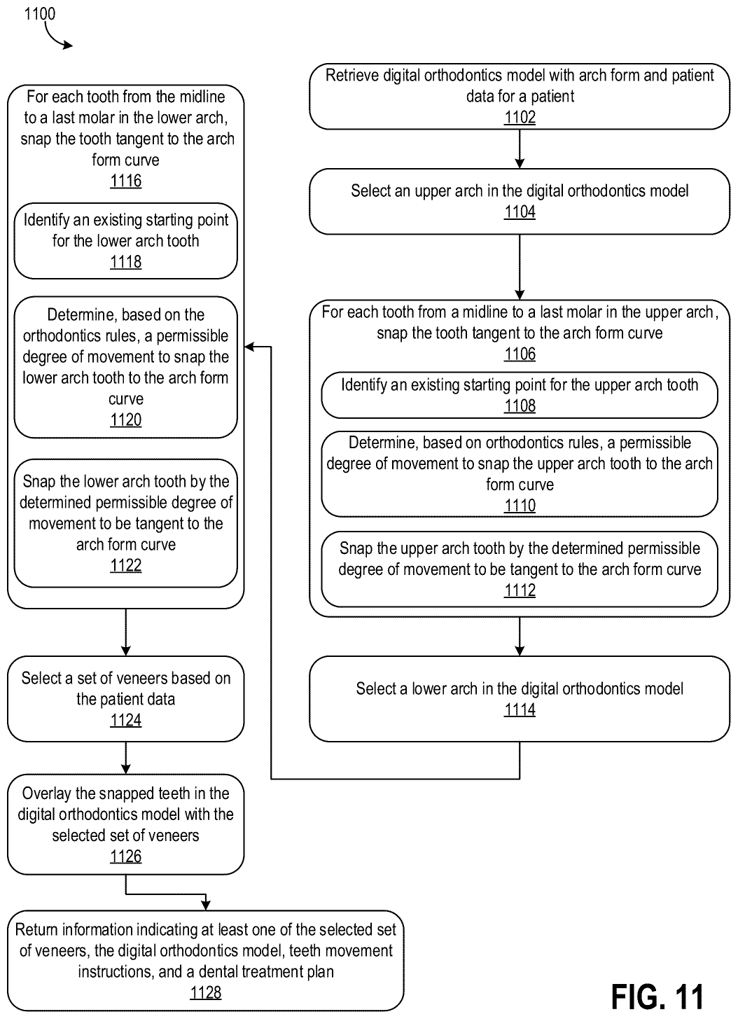

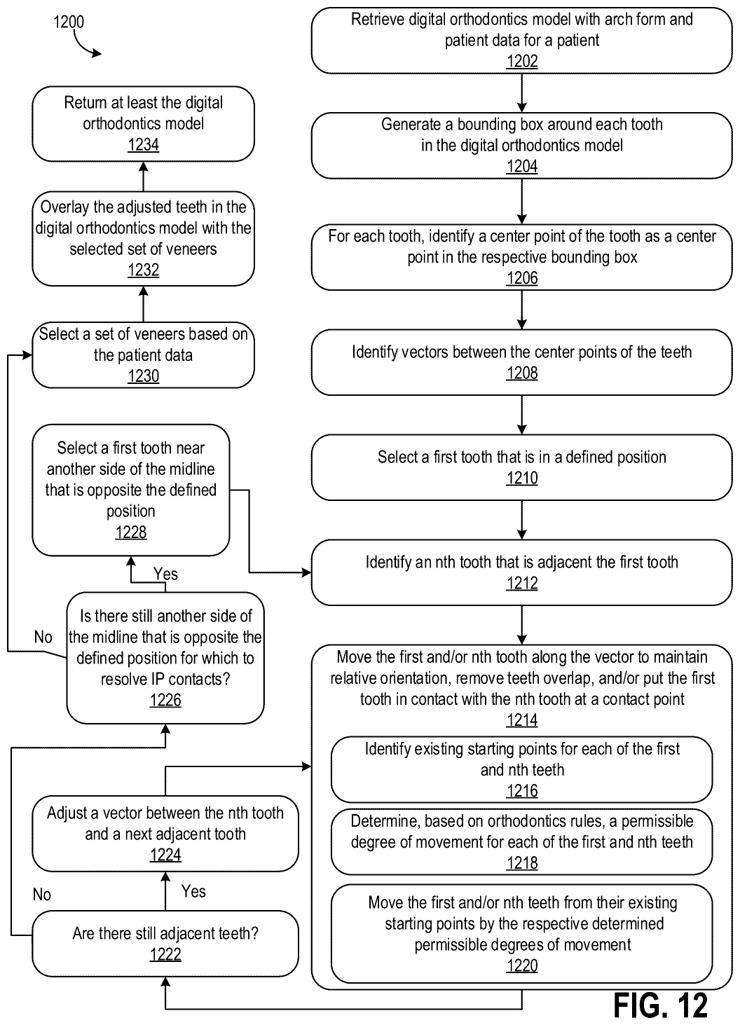

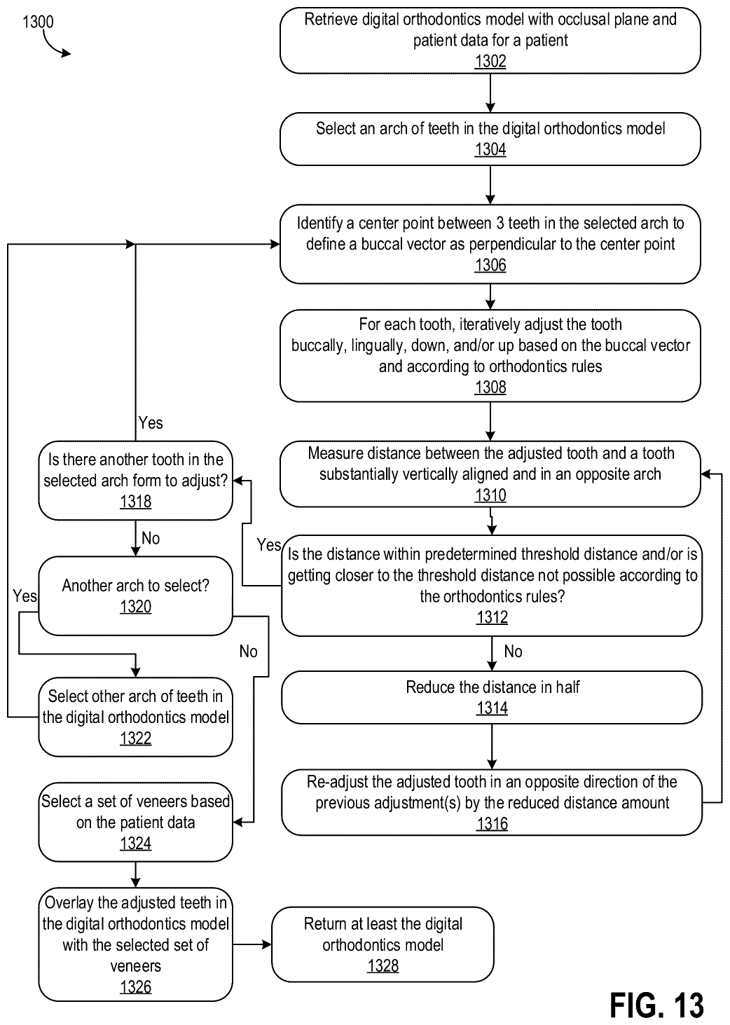

The computer system 102 can determine tooth movements along the arch form in the digital model to achieve the desired smile design (e.g., teeth arrangement) for the patient and based on the orthodontics rules (block G, 132). For example, the computer system 102 can level the teeth in upper and/or lower arches of the model based on identifying datums for each tooth and/or relative an occlusal plane. Individual teeth can be leveled by the computer system 102 so long as the leveling movements are within a threshold range of permissible leveling movement for the particular tooth, as defined by the orthodontics rules. Refer to FIG. 10 for further discussion about leveling the teeth. As another example, the computer system 102 can snap the teeth in the upper and/or lower arches of the model to the defined arch form, so long as the snapping movements are within a threshold range of permissible snapping movement for the particular tooth, as defined by the orthodontics rules. Refer to FIG. 11 for further discussion about snapping the teeth. As yet another example, the computer system 102 can resolve any interproximal (IP) contacts between adjacent in the model by moving the adjacent teeth relative each other in a 2D direction. The computer system 102 can perform these movements so long as they are within a threshold range of permissible contact movement for the particular tooth and/or adjacent teeth, as defined by the orthodontics rules. Refer to FIG. 12 for further discussion about resolving IP contacts. Sometimes, the computer system 102 can adjust vertical positioning of one or more teeth in the upper and/or lower arches of the model. The computer system 102 can adjust the vertical positioning so long as these movements are within a threshold range of permissible vertical movement for the particular tooth, as defined by the orthodontics rules. In some implementations, for example, teeth may not be repositioned up and down (e.g., vertically) when designing and selecting veneers. Refer to FIG. 13 for further discussion.

The computer system 102 can select a set of veneers from the data store 106 for designing the patient's smile, based on the tooth movements and/or the patient tooth data (block H, 134). The computer system 102 can select a set of veneers that match a desired tooth shape and/or appearance for the patient. Additionally or alternatively, the computer system 102 can select a set of veneers that have a shape and/or size that allows the veneers to be attached to the patient's teeth once the teeth are moved according to the determined tooth movements. Refer to blocks 508-514 in process 500 of FIG. 5 for further discussion about selecting veneers based on tooth shape information.

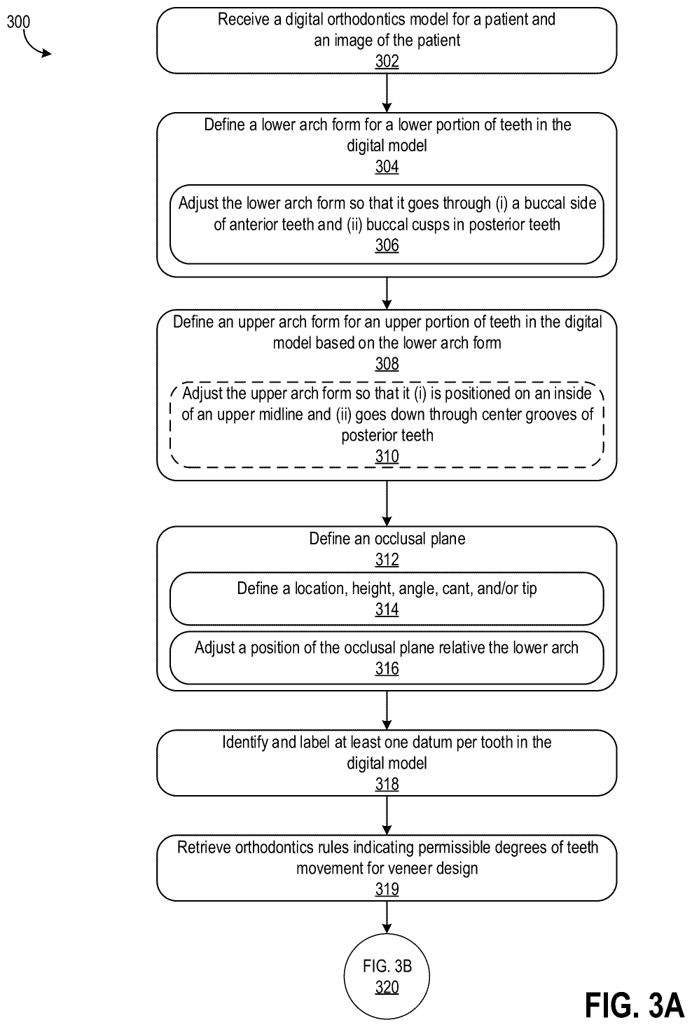

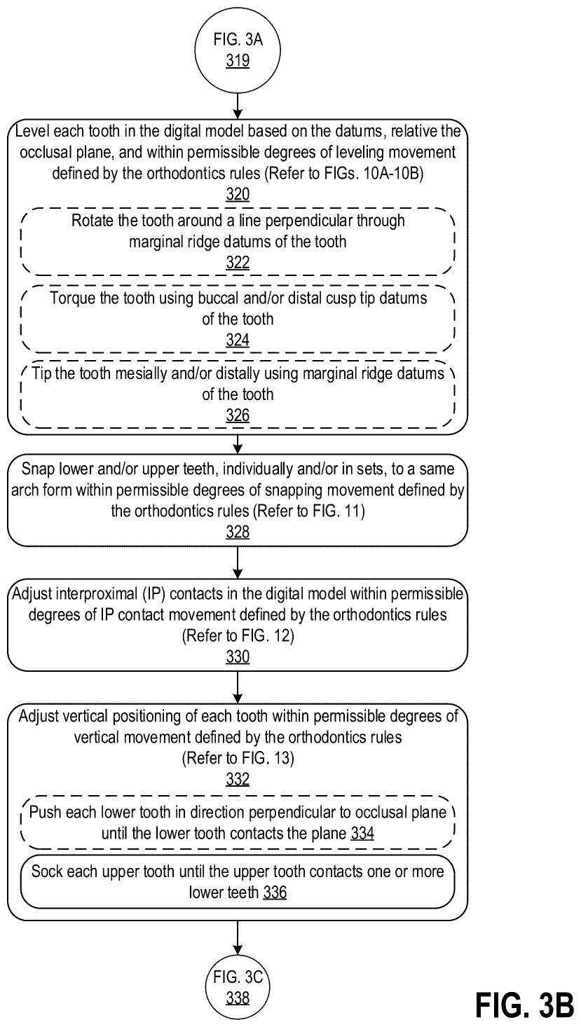

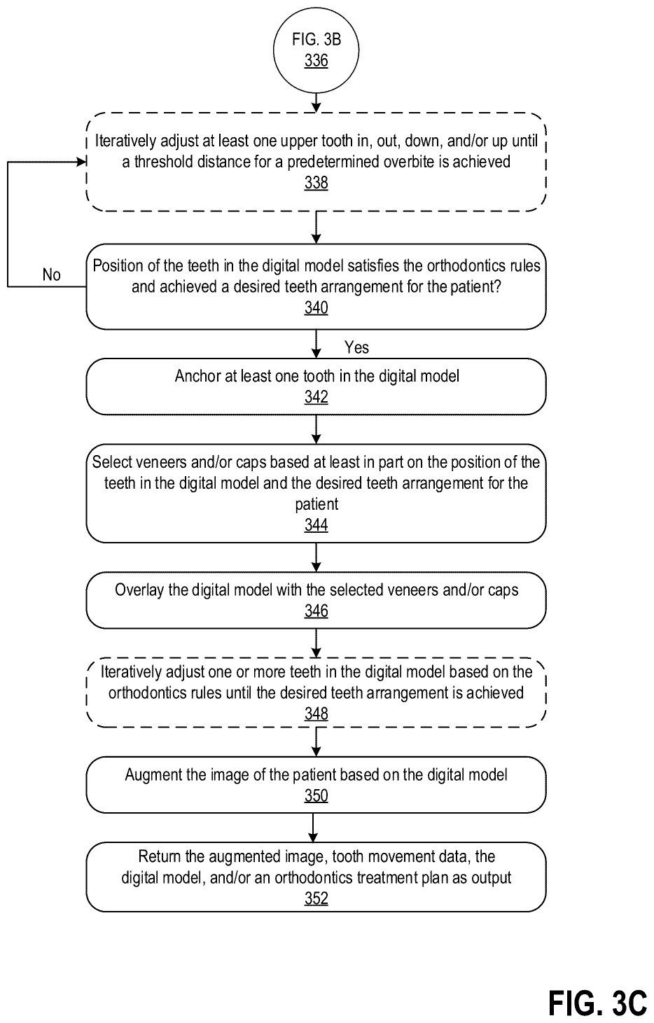

In some implementations, the computer system 102 can perform techniques such as measuring teeth in the patient tooth data and using those measurements to identify sets of veneers in the data store 106 having similar tooth measurements (or measurements within a threshold range of the measurements for the particular patient). The computer system 102 can select a candidate set of veneers amongst a plurality of sets of veneers based on identifying a best fitting set of veneers for the particular patient using machine learning models and/or artificial intelligence (AI) algorithms. The computer system 102 can score each of the sets of veneers and select a highest-scoring set as the candidate set of veneers. The computer system 102 can additionally or alternatively consider tooth shape, tooth lengths, tooth widths, and/or aggregate tooth measurements in selecting the candidate set of veneers to achieve the desired smile design for the patient. Refer to at least FIGS. 3A, 3B, and 3C for further discussion.

In block I (136), the computer system 102 can overlay the digital model with the selected set of veneers. The set of veneers can be visualized and displayed based on the determined tooth movements. For example, the veneers can overlay the moved teeth in the model.

Optionally, the computer system 102 can iteratively adjust one or more teeth in the upper and/or lower arches of the model based on the orthodontics rules and until the desired teeth arrangement is achieved for the patient (block J, 138). Block J can be performed as part of block G (132). In some implementations, block J can be performed in response to overlaying the digital model with the selected veneers in block I (136) and determining, by the computer system 102, that the teeth of the model and the selected veneers are not aligned. As another example, block J can be performed in response to determining, by the computer system 102, that the selected veneers work for the patient's desired teeth arrangement but that the patient's teeth need to be moved a little more to achieve the desired teeth arrangement.

The computer system 102 can augment the image 148 of the patient based on the digital model to generate an augmented image 149 (block K, 140). The computer system 102 can perform image processing techniques to generate the augmented image 149 from the image 148. For example, the computer system 102 can adapt the image 148 to include the veneers from the digital model, as the veneers are arranged relative to teeth movements in the patient's mouth. The augmented image 149 can advantageously provide a realistic visual of what the patient would look like if their teeth are moved according to the teeth movements and the selected set of veneers are applied to their teeth.

The computer system 102 can return the augmented image 149, tooth movement data, and/or an orthodontics treatment plan as output in block L (142). Returning any of these outputs can include storing the output(s) in the data store 106 for later retrieval and/or use by the computer system 102 (e.g., in generating or adjusting digital orthodontics models for the patient in the future, for iteratively training one or more machine learning models used for auto-designing smiles) and/or the user device 108 (e.g., in viewing and modifying the model for the patient, in approving the model and sending instructions to one or more relevant users for implementing the orthodontics treatment plan and/or veneer installation process). In the example of FIG. 1A, returning the output includes transmitting the output to the user device 108.

The user device 108 can present any of the returned output in one or more GUIs presented in a display of the user device 108 (block M, 144). The relevant user, such as a technician, may choose to adjust one or more of the teeth in the digital orthodontics model, select one or more different sets of veneers for the patient, modify the orthodontics treatment plan, etc. Any of these actions can be provided at the user device 108 as user input and transmitted to the computer system 102. The computer system 102 can auto-adjust the teeth in the model multiple times in response to and based on the user input (e.g., ensuring that when the user moves one tooth, adjacent teeth are also appropriately adjusted to avoid collision or interference while ensuring that the teeth are moved within the permissible degrees of movement defined by the orthodontics rules). Accordingly, the computer system 102 can iteratively perform one or more of the blocks F-K (132-140) in response to the user input.

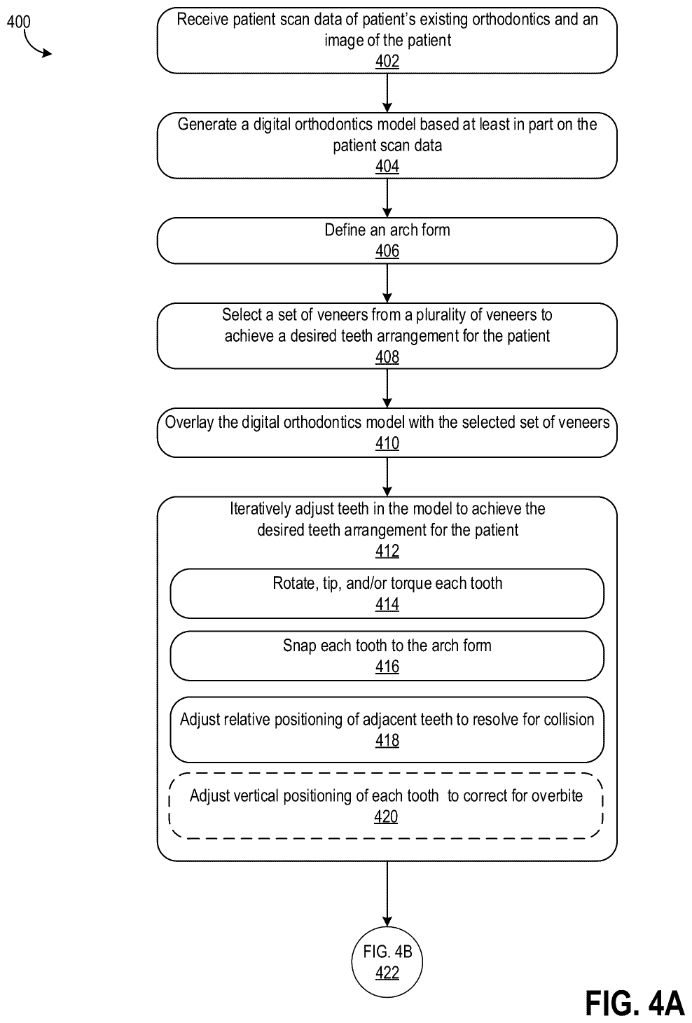

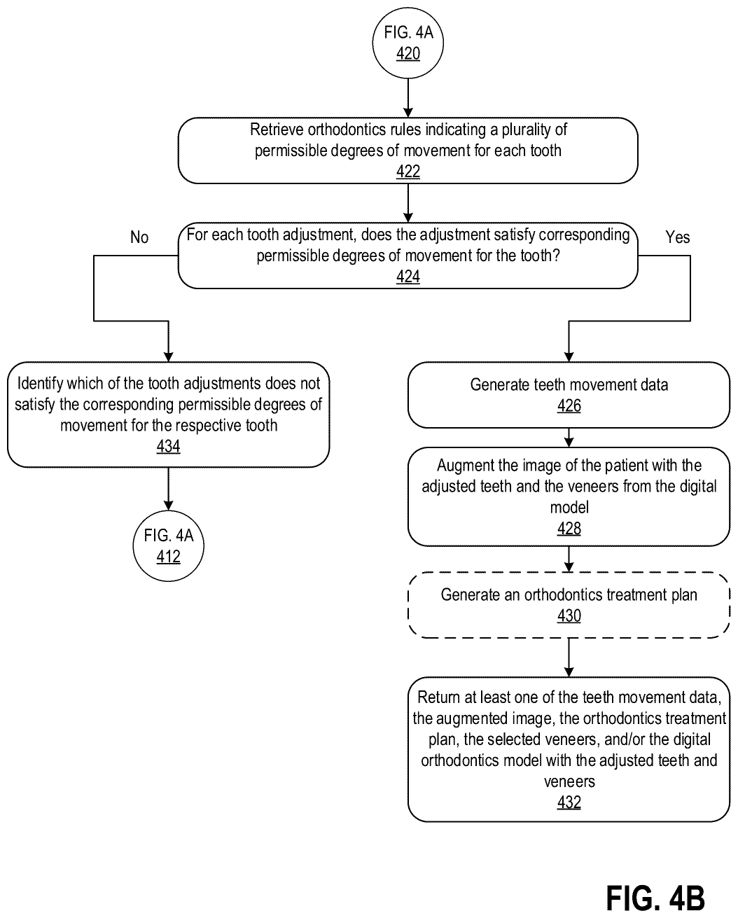

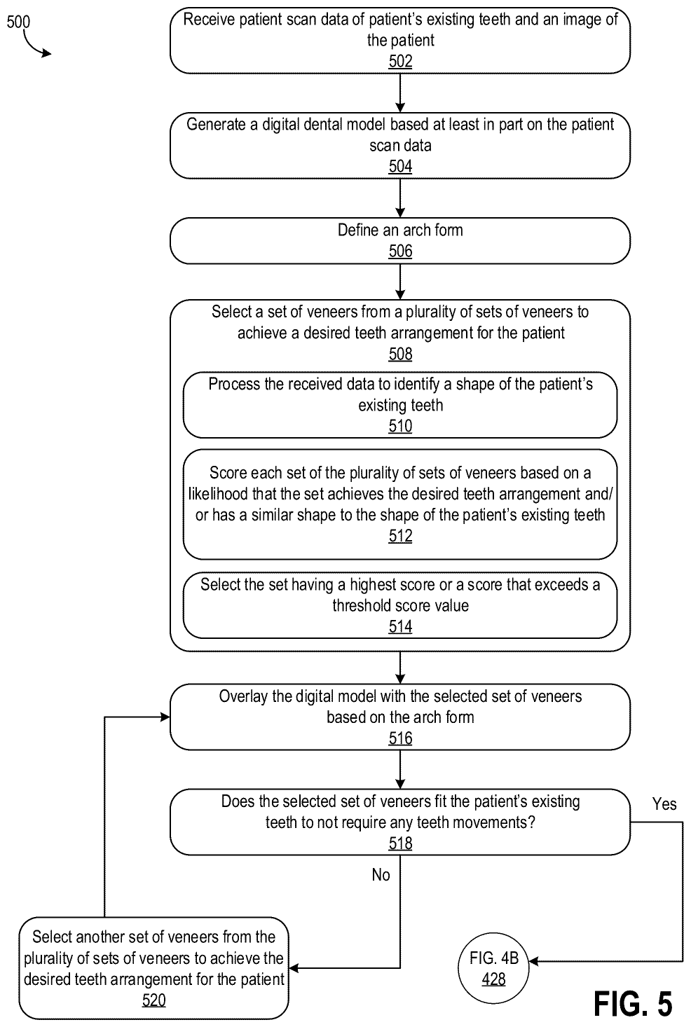

FIG. 1B is a conceptual diagram of the system 100 for computer-automated design of veneers for a patient based on determining whether permissible teeth movements can be made to achieve a look created by the veneers. Whereas FIG. 1A illustrates techniques for selecting veneers based on tooth movements that have been made to a digital orthodontics model for the patient, FIG. 1B illustrates techniques for selecting veneers, and then determining permissible tooth movements for achieving the look created by the selected veneers. The techniques described in FIG. 1B are further described in FIGS. 4A and 4B. In some implementations, the techniques described in reference to FIG. 1B can also be used to select veneers and determine whether a desired teeth arrangement for the patient is achieved with the selected veneers and without requiring the patient's teeth to be moved. If, for example, the selected veneers would require movement of the patient's teeth to achieve the desired teeth arrangement, then a different set of veneers can be selected and techniques described herein can be iteratively performed until a best set of veneers are identified for the patient.

Referring to FIG. 1B, the scanning devices 104A-N can scan patient tooth data in block A (150). The scanning devices 104A-N can transmit the patient tooth data 146 to the computer system 102 (block B, 152). The computer system 102 can also receive the patient image data 148 in block C (154). In block D, the computer system 102 can retrieve one or more orthodontics rules from the data store 106 (block D, 156). The computer system 102 can generate a digital orthodontics model based on the patient tooth data in block E (158). The computer system 102 can define an arch form in the model (block F, 160). Refer to blocks AF (120-130) in FIG. 1A for further discussion.

In block G, the computer system 102 can select a set of veneers based at least in part on the patent tooth data (162). The computer system 102 can, for example, identify a set of veneers that provide a best visual appearance to achieve the patient's desired tooth arrangement. The computer system 102 can identify a set of veneers that may best fit the patient's mouth and/or existing teeth surfaces. The computer system 102 can identify a set of veneers that may achieve the desired tooth arrangement for the patient with minimal tooth movements. One or more other selection criteria can be used by the computer system 102 to select a set of veneers. Refer at least to FIGS. 4A and 4B, and block H (134) in FIG. 1A for further discussion.

The computer system 102 can then overlay the digital model with the selected set of veneers in a desired teeth arrangement (block H, 164). For example, the selected set of veneers can be positioned over teeth in the model that have not yet been aligned or moved in order to achieve the desired teeth arrangement for the patient. Therefore, the computer system 102 can place the veneers in the desired tooth arrangement for the patient and then determine what tooth movements are required to achieve this desired arrangement and whether those tooth movements are in fact permissible in light of threshold ranges of teeth movement defined in the orthodontics rules.

The computer system 102 can determine one or more tooth movements along the arch form in the digital model to achieve the desired teeth arrangement for the patient and satisfy the orthodontics rules (block I, 166). As mentioned above, the computer system 102 can determine what movements ought to be made to achieve the placement of the veneers in the digital model. Refer to block G (132) in FIG. 1A for further discussion.

In block J, the computer system 102 can optionally iteratively adjust the teeth in the digital model based on the determined tooth movements (168). Block J (168) can be performed as part of block G (132). Refer to block J (138) in FIG. 1A for further discussion.

The computer system 102 can also augment the image 148 of the patient based on the digital model to generate the augmented image 149 (block K, 170). The computer system 102 can return the augmented image, tooth movement data, and/or an orthodontics treatment plan as output in block L (172). The user device 108 can, for example, present the output or a portion thereof in a display at the user device 108 (block M, 174). Refer to blocks K-M (140-144) in FIG. 1A for further discussion.

FIG. 1C is a block diagram of system components for computer-automated smile design and/or orthodontics treatment planning described herein. The digital smile design computer system 102 can include a perfect smile setup engine 2202, an image augmentation engine 180, and orthodontics rules 182A-N.

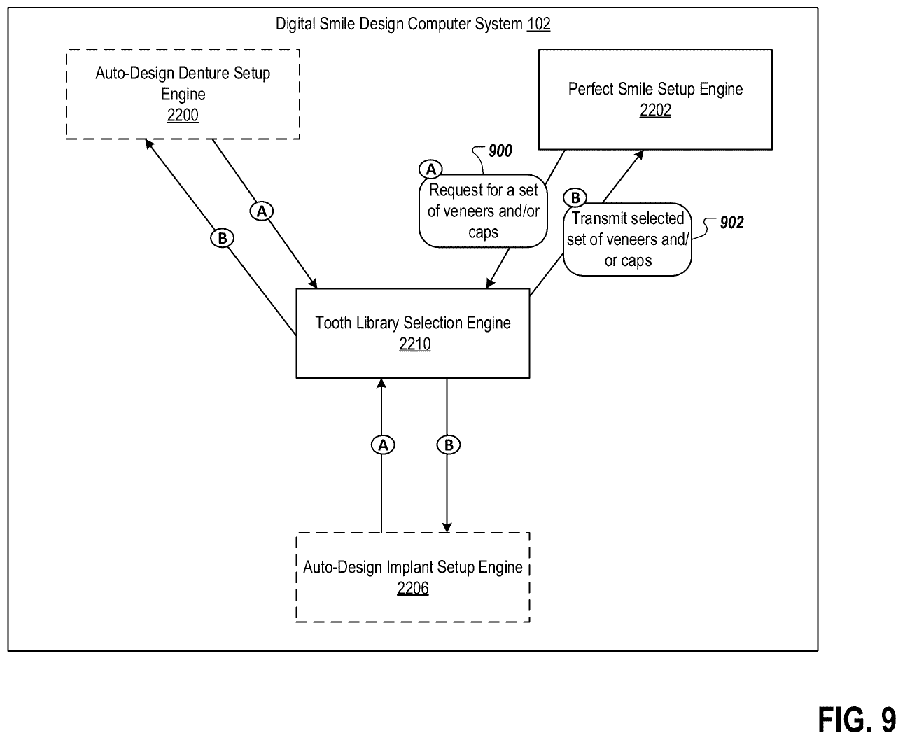

The perfect smile setup engine 2202, further referenced and described in FIG. 9, can be configured to generate a digital orthodontics model, select a set of veneers for a patient, and determine appropriate teeth movements in the model to achieve a desired teeth arrangement for the patient with the set of veneers. The engine 2202 can further design the patient's smile using the orthodontics rules 182A-N, described further in reference to FIG. 1A. The engine 2202 can receive the tooth scan data 146 described in FIG. 1A and generate the digital model with the set of veneers and tooth movements based on the tooth scan data 146. The engine 2202 can generate tooth movement data 186 once the auto-smile design process is complete (e.g., the desired teeth arrangement is achieved and any required tooth movements are within threshold ranges of permissible movement defined by the orthodontics rules 182A-N). The tooth movement data 186 can be transmitted to an orthodontics planning system 184.

The orthodontics planning system 184 can be any type of computing system described herein that can be configured to determine a treatment plan 188 for the patient. The treatment plan 188 can include instructions for directing a technician, orthodontist, or other relevant user to adjust the patient's teeth according to the treatment plan 188. For example, the treatment plan 188 can include instructions for designing and/or placing braces, retainers, aligners, or other orthodontic dental appliances in the patient's mouth. In some implementations, the orthodontics planning system 184 can be part of the computer system 102 and/or part of the user device 108.

The treatment plan 188 can also be transmitted to the user device 108. The user device 108, as described in reference to FIG. 1A, can provide a patient/doctor, patient/technician, or other relevant user interface for presenting information, such as the GUIs described herein. The user device 108 can, for example, output the treatment plan 188 in a GUI. The relevant user can review the treatment plan 188 and make one or more modifications. The modifications can be sent back to either of the systems 102 and 184. The modifications can be used to iteratively improve either of the systems 102 and 184 in generating subsequent treatment plans for the particular patient and/or for other patients. In some implementations, the user device 108 can generate the treatment plan 188, then transmit the plan 188 to the system 184 for execution.

Referring back to the perfect smile setup engine 2202, when the engine 2202 selects a set of veneers or other orthodontic dental appliances such as caps or crowns to be used to enhance the patient's smile, the engine 2202 can transmit a veneer/cap tooth selection 190 to the user device 108. The user device 108 can output the selection 190 in a GUI. The relevant user can review the selected veneers and decide whether to approve the veneers or select a different set of veneers. Any modifications made to the veneer selection by the user at the user device 108 can be transmitted back to either of the systems 102 and 184. The system 102 can use the user modifications to perform one or more iterative adjustments on teeth in the model to better place them for receiving the user-selected veneers. The system 184 can use the user modifications to adjust the treatment plan 188 accordingly.

The image augmentation engine 180 of the computer system 102 can be configured to apply image processing techniques to the image 148 of the patient and augment the image with the selected veneers 190. The engine 180 can generate the augmented image 149 of the patient, which is described further in reference to FIG. 1A. The augmented image 149 can be transmitted to the user device 108 and presented in a GUI. The relevant user and/or the patient can review the augmented image 149 and decide whether the desired teeth arrangement is achieved. If so, the users can approve the auto-smile setup and the users can proceed according to the treatment plan 188.

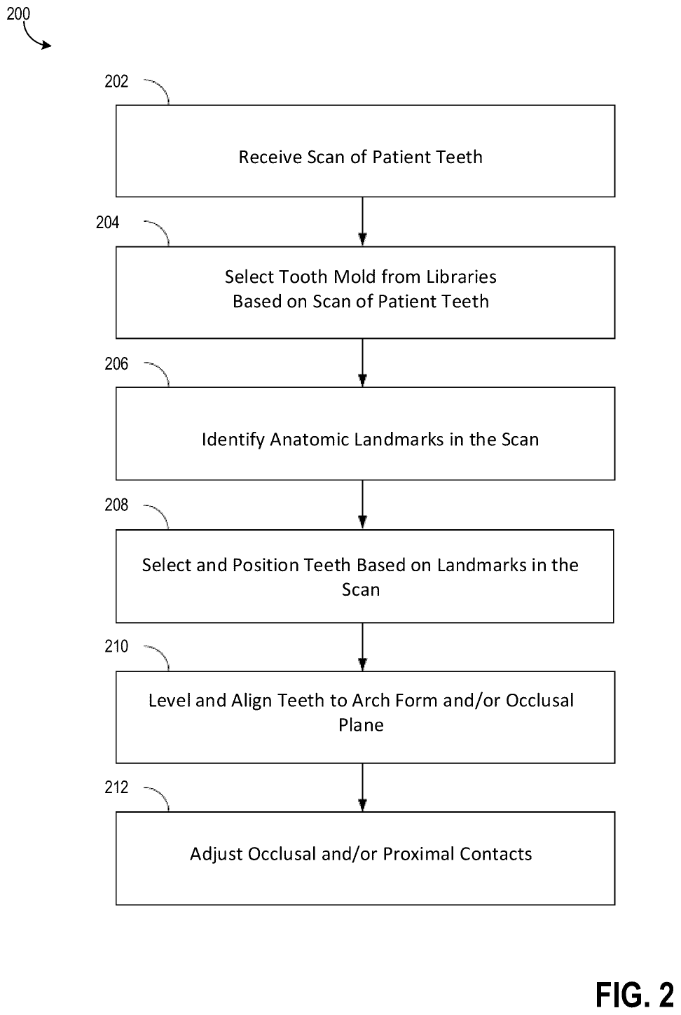

FIG. 2 is a flowchart of an example process 200 for automatic smile design. The process 200 can be performed by the digital smile design computer system 102 described in reference to at least FIG. 1A. The process 200 can also be performed by any other type of computer system, computing device, cloud-based system, and/or network of computing systems. For illustrative purposes, the process 200 is described from the perspective of a computer system.

Referring to the process 200 in FIG. 2, at block 202, a scan of a patient's teeth is received by the computer system. In some implementations, the scan can be produced using a CT scanning device, a CBCT scanning device, an intraoral scanner, or a desktop scanner. In some implementations, the scan is processed by the computer system upon receipt, for example by detecting and filling in voids in the scan, removing scan artifacts arising from metallic inclusions in dentures (if the patient is wearing dentures at the time of scanning and/or their dentures are scanned), correcting holes in the scan arising from positioning of the dentures in a jig or on a surface during the scan, thresholding the scan data to produce a surface scan, or converting a file type of the scan to another file type. For example, in some implementations, the scan data is converted from the native file type of the CT scanner (for example, a Digital Imaging and Communications in Medicine (DICOM) file) to a data model file type such as a stereolithography (STL) file type. In some implementations, the DICOM file or other native file of the CT scanner is converted to a polygon file format (PLY), a standard 3D image file format (OBJ), an additive manufacturing file format (AMF), a 3D manufacturing file format (3MF), or any other suitable file type.

In some implementations, additional digital patient data can also be received, for example motion data or color images of the patients teeth, or other image data of the patient as described in reference to FIG. 1A. Motion data can be captured using a motion capture system and can represent the patient's jaw moving through various jaw movements for use in designing the patient's smile. Color images can be obtained simultaneously with the scanning of the patient's teeth and can be used to overlay color on the smile design and/or determine color of veneers for the patient's smile design. Additional details related to the use of motion capture data and color images can be found in U.S. Provisional Patent Application Ser. No. 63/149,178 filed on Feb. 12, 2021 and entitled “Motion-Based Digital Denture Design,” U.S. Provisional Patent Application Ser. No. 63/274,798 filed on Nov. 2, 2021 and entitled “Digital Denture Design and Replacement,” the PCT Application filed Feb. 10, 2022 and entitled “Digital Denture Design and Replacement,” and in U.S. Provisional Patent Application Ser. No. 63/313,723 filed on Feb. 24, 2022 and titled “Color Digital Denture Design and Replacement,” the contents of each of which is incorporated herein by reference in its entirety.

At block 204, a tooth mold can be selected by the computer system from a tooth library and based on the scan of the patient's teeth. As described in reference to FIG. 1A, the tooth molds can include sets of veneers that have been predefined and stored in a static data store. The sets of veneers can be accessed and reviewed by the computer system to select a tooth mold having a best fit to achieve a desired teeth arrangement/smile design for the patient. The sets of veneers to choose from may vary functionally, aesthetically, and/or based on manufacturer. The software can use a number of mechanisms for automatically selecting and scoring the match between the patient's teeth and the tooth mold options in one or more libraries of teeth, including but not limited to a software scoring scheme that determines a perfect or preferred or best match or closest alternative from available libraries or set of libraries.

In some implementations, selection of the tooth mold is made by the computer system based on landmarks and anatomical dimensions of a subset of teeth of the scan and the selected tooth mold can be applied for all teeth for the patient. For example, a width of a particular tooth can be used as an initial selection criteria for determining a best matching tooth mold from the library. In some implementations, the width of one or more teeth can be used to identify a subset of candidate libraries, and an iterative fit algorithm or other mechanism can be used to determine a best or preferred library from the subset of candidate libraries. These operations can advantageously reduce an amount of processing power required for determining a best-fit library compared to using an iterative algorithm across a large number of potential libraries.

In some implementations, the selection of the tooth mold can be made for each individual tooth based on the landmark and anatomical dimensions of the particular tooth. In some implementations, the selection of the tooth mold is made based on a tooth or a sub set of teeth in upper teeth, and the selected tooth mold that is the perfect or closest match is then applied to lower teeth. In some implementations, the selection of the tooth mold can be independent for the upper and lower teeth. The selection of the tooth mold may also take into account the landmarks and dimensions of teeth on both the upper and lower teeth. As a result, the upper and lower teeth can be positioned relative to each other to provide improved overall appearance of the patient's smile.

Sometimes, additional information including but not limited to patient specific motion from motion capture scans and/or images and hinge axis information (e.g., from measuring jaw movement of the patient) can also be accounted for in the computer-automatic matching and selection of the tooth mold from one or more tooth libraries.

The selected tooth mold may be displayed visually so that a user may confirm or reject the selection. The closest or perfect match can be automatically selected and presented in a GUI, as described further below. Alternatively or additionally, top matches can be presented in the GUI for user selection. In some implementations, several candidate sets of veneers (e.g., three that each has a closest width to that of the patient's digital orthodontics model) can be selected and presented to a user. In some implementations, the closest match can be presented with one, two, three, or four next closest matches for user confirmation or selection. A user may use a GUI to select between these options. In some implementations, the closest match and other next closest matches can be presented separately and/or in an overlay presentation using a color map to illustrate differences across the different tooth molds, as described further below.

Enabling the automatic matching of tooth landmarks to an existing library tooth mold can efficiently identify best teeth for the patient's smile design. The speed with which the computer system can identify a match using an iterative fitting algorithm, such as iterative closest point (ICP) or another algorithm, is much greater than the speed with which a technician can determine an appropriate library of teeth. The disclosed technology can further generate statistics and visualizations to quantify a fit of the tooth mold from the library to the patient's teeth, and the information can be presented to a technician for approval or adjustment to patient specifications, desires, or dental requirements.

At block 206, anatomic landmarks can be identified in the scan. The identification of individual teeth can be determined, for example, molars, pre-molars, canines, and incisors. Additionally, a width of each tooth or of particular teeth can be determined by the computer system. The height, circumference, or cross-section can also be determined. Other landmarks such as cusps, pits, offsets, and grooves can be identified in the scan. The identified landmarks can be presented to the technician through the GUIs described herein.

The GUI can include a presentation field in which the digital orthodontics model can be displayed and manipulated by the user. The user can interact with the digital model by clicking and dragging, using arrow keys, using a joystick, or by using any other user selection interface. The GUIs can include a toolbox for displaying options for manipulation, selection, display, and adjustment of the digital model displayed in the user interface. In some implementations, the GUI can include a specialized process flow for guiding the user through the design process in a particular order.

In some implementations, the computer system can identify missing portions of a scan, for example if a tooth is broken off, the computer system identifies this as a discrepancy and can alert a user, such as the technician, through the user interface and/or suggest a tooth for positioning in the missing portion, based on selection of other teeth to fit the digital model and further based on the scan data.

Still referring to FIG. 2, at block 208, the teeth from the selected tooth mold can be selected and positioned on the scan based on the identified landmarks and other tooth morphology. For example, a veneer tooth from the library tooth mold can be selected by the computer system for a particular tooth of the scan and based on a shape matching of the teeth and/or based on a labeling of the library tooth mold teeth and labeling applied to the scan based on the identified landmarks. Refer to at least blocks 508-514 in the process 500 of FIG. 5 for further discussion about selecting veneers based on teeth shape. In some implementations, the digital teeth can be positioned based on a determined or selected occlusal guidance surface. In some implementations, the digital teeth may include labels for anatomical landmarks such as cusps, marginal ridges, incisal edges, fossa, grooves, base boundaries, and/or other anatomical landmarks. These labels may be used to automatically position the digital teeth with respect to one another and digital teeth on opposing arches (e.g., upper and lower arches).

The digital teeth may be initially positioned in alignment with an arch curve. The arch curve may be sized and shaped based on the digital orthodontics model. Refer to the process 300 in FIGS. 3A, 3B, and 3C for additional discussion. Each of the digital teeth may include one or more labels that specify one or more locations on the digital teeth to align to the arch curve. The digital teeth may also be associated with a tip and torque with respect to one or more of the arch curve and an occlusal plane. When initially positioned, the digital teeth may be positioned with respect to the arch curve based on the labels and automatically tipped and torqued with respect to the arch curve based on the associated values. As described herein, the digital teeth can be positioned, tipped, and/or torqued within threshold ranges of permissible movement as defined for each tooth in orthodontics rules (refer to FIG. 1A for further discussion about the orthodontics rules).

Once the digital teeth are in their initial positions, their positions may be further refined. At block 210, the teeth can be leveled and aligned to the arch form and to the occlusal plane by the computer system. Once selected, the tooth from the library tooth mold can be automatically positioned in the scan so as to most closely match the tooth placement in the existing patient's teeth. This can include leveling the teeth and aligning the teeth on the arch. For example, an iterative closest point (ICP) algorithm can be used to determine the appropriate position of the library tooth mold tooth to match the tooth in the existing patient's teeth. An ICP algorithm can determine not only a general “best-fit” but can determine the best-fit within microns. Refer to FIGS. 3A, 3B, 3C, 10, 11, 12, and 13 for further discussion about adjusting the teeth to achieve a desired teeth arrangement for the patient while working within constraints of permissible tooth movements as defined by the orthodontics rules.

After the teeth are automatically adjusted to fit the arch and the digital scan, the scan including the library tooth mold teeth can also be presented in the GUIs to enable the technician to manipulate and adjust the teeth further. At block 212, the occlusal and proximal contacts can be adjusted by the computer system. The GUI may receive user input to move a selected digital tooth. In some implementations, the user input can include a drag input such as a click-and-drag or touch-and-drag. Based on a direction of the drag, the digital tooth may move in a corresponding direction. In some implementations, the movement may be in a direction that is parallel to the occlusal plane. In some implementations, as the digital tooth moves based on the drag input, the digital tooth also can move in an occlusal-gingival direction to make contact with the opposing dentition. In some implementations, the digital tooth may be moved to contact with an occlusal guidance surface that is generated based on opposing teeth and motion data (e.g., by sweeping the opposing teeth through the motion of the motion data).

Using collision informed design mechanisms, the teeth can be automatically positioned by the computer system such that adjacent teeth avoid interference or overlap with one another. The teeth can behave as real teeth while they are adjusted to the digital scan. Using real-time collision detection algorithms, the surface of the tooth can be moved with respect to other teeth in the area by the computer system. The teeth can be moved in multiple directions, including vertically and horizontally, and can be twisted or tilted to adjust the contacts and positioning. The upper and lower teeth can also be manipulated relative to one another automatically by the computer system and/or manually by the technician or other relevant user. Individual teeth, groupings of teeth, or entire upper or lower teeth can be adjusted, so long as the adjustments are permissible according to the orthodontics rules described herein.

One or more teeth can be moved together by the computer system to adjust occlusal and/or proximal contacts. Using collision avoidance, the digital teeth behave like actual teeth when they are adjusted in that the teeth cannot pass through each other but are instead restricted in their movement by adjacent teeth. For example, when one tooth is selected and adjusted, it can be bounded by a tooth on either side and can only move to be adjacent the teeth but cannot move through the teeth or into the teeth. Similarly, when multiple of the upper teeth are selected for adjustment together, the selected teeth can maintain their original spacing but can move up or down to maintain contact with teeth of the lower teeth.

Following block 212, the smile design can be finalized by the computer system. In some implementations, the smile design is presented in the user interface for approval and can then be used to fabricate the replacement dentures.

Automating the design process as described herein can increase efficiency of smile design by reducing design aspects that a technician must complete from scratch, manually, and/or visually. Providing suggestions of sets of veneers (e.g., tooth libraries), landmark identifications, and tooth positions/movements can reduce the amount of work and time required from a technician, thereby accelerating the process and reducing costs associated with designing smiles and orthodontic treatment plans. Rather than begin the design from scratch and prepare the smile design based on visual inspection of the patient's scan, the computer system can make suggestions based on advanced and complex best-fit algorithms to produce a smile design that closely matches the patient's desired teeth arrangement in fit, function, and appearance. Such high-quality smile designs from automation of the design process can reduce the time to design and also reduce the cost of developing the smile design while minimizing differences in smiles designed by various technicians for improved patient experience.

FIGS. 3A, 3B, and 3C are a flowchart of a process 300 for computer-automated smile design for a patient. The process 300 is similar to the operations described in FIG. 1A. In other words, the process 300 includes operations for designing the patient's smile by selecting veneers for the patient based on performing permissible degrees of movement on the patient's teeth, as defined by orthodontics rules.

The process 300 can be performed by the digital smile design computer system 102 described in reference to at least FIG. 1A. The process 300 can also be performed by any other type of computer system, computing device, cloud-based system, and/or network of computing systems. For illustrative purposes, the process 300 is described from the perspective of a computer system.

Referring to the process 300 in FIGS. 3A, 3B, and 3C, the computer system can receive a digital orthodontics model for a patient and an image of the patient in block 302. The model can be generated as described at least in reference to FIG. 1A. In some implementations, the model can be generated as part of the process 300. Refer to FIG. 1A for further discussion.

The computer system can define a lower arch form for a lower portion of teeth in the digital model in block 304. For example, the computer system can adjust the lower arch form so that it goes through (i) a buccal side of lower anterior teeth and/or (ii) buccal cusps in lower posterior teeth (block 306). In some implementations, the computer system can receive user input from a user device of a relevant user, the user input indicating one or more adjustments to be made to the lower arch form. The arch form can be defined by the computer system and therefore used for which to align teeth of the digital model.

In block 308, the computer system can define an upper arch form for an upper portion of teeth in the digital model based on the lower arch form. The computer system may optionally adjust the upper arch form so that it (i) is positioned on an inside of an upper midline of upper anterior teeth and/or (ii) goes down through center grooves of upper posterior teeth (block 310). The upper arch form can be the same as the lower arch form. Accordingly, the computer system can mirror the lower arch form for the upper teeth. The computer system can also make adjustments to the upper arch form once the lower arch form is mirrored/replicated for the upper teeth. For example, the computer system can move the arch form so that it appropriately aligns with upper anterior teeth. In some implementations, the computer system can receive user input indicating one or modifications to shape and/or placement of the upper arch form.

The computer system can also define an occlusal plane in block 312. For example, the computer system can define a location, height, angle, cant, and/or tip of the occlusal plane relative the teeth in the digital model (block 314). As another example, the computer system can adjust a position of the occlusal plane relative to the lower arch (or optionally the upper arch) (block 316). The computer system can move the occlusal plane up and/or down until (for example) tips of the lower anterior teeth touch and/or slightly pass through the occlusal plane. Sometimes, a curve of Wilson (from an anterior view) and/or a curve of spee (from a side view) can be used by the computer system to curve the occlusal plane, especially for purposes of designing the patient's smile. For example, the computer system can define the occlusal plane to curve upward from the anterior view such that buccal cusp tips may be lower than lingual cusp tips. In some implementations, the computer system can receive user input indicating one or more adjustments to the occlusal plane.

In block 318, the computer system can identify and label at least one datum per tooth in the digital model. The computer system can apply one or more machine learning models to the digital model. A model can, for example, be trained to identify and label a plurality of different types of datums, landmarks, or other types of markers for different types of teeth in the digital model. The computer system can use an automated algorithm for identifying the plurality of datums in the digital model. The user can provide user input to adjust placement of one or more of the identified datums. The computer system can identify and label datums that include but may not be limited to edges of cusp tips, marginal ridge points, molars, low spots between cusps where adjacent teeth may touch, canine cusp tips, etc.

As an illustrative example, the computer system can apply an algorithm or a model that has been trained to identify datums on incisors in the model. The incisor datums can include one or more datums on each incisal edge (e.g., 2 datums on an incisal edge). A biting surface of the incisor can, for example, have 2 datums. As another example, the computer system can identify at least one datum on a cusp tip for each canine in the model. For bicuspids and molars, the computer system can identify at least one datum on each cusp tip and/or marginal ridge point per bicuspid and molar. Various other datums can be identified and labeled by the computer system, as described throughout this disclosure.

In block 319, the computer system can retrieve orthodontics rules indicating permissible degrees of teeth movement for veneer design. The rules can be retrieved from a data store. In some implementations, the rules can be locally stored in memory at the computer system and quickly accessed in block 319. Refer to FIG. 1A for further discussion about the orthodontics rules and the permissible degrees of teeth movement for veneer design.

The computer system can level each tooth in the digital model based on the datums and relative to the occlusal plane (block 320). The leveling per tooth can be performed within permissible degrees of leveling movement defined by the retrieved orthodontics rules for the particular tooth. For example, the computer system may level an incisor tooth by torqueing the tooth within a range of movement that is permissible (e.g., safe, allowed, accepted, appears aesthetically pleasing) for an incisor. The computer system may not torque the tooth beyond the range of permissible movement for the incisor. Refer to FIGS. 10A and 10B for further discussion about leveling the teeth.

In some implementations, the computer system can optionally rotate the tooth around a line perpendicular through marginal ridge datums of the tooth (block 322). Additionally or alternatively, the computer system can optionally torque the tooth using buccal and/or distal cusp tip datums of the tooth (block 324). Additionally or alternatively, the computer system can optionally tip the tooth mesially and/or distally using marginal ridge datums of the tooth (block 326). As mentioned above, each of these types of movements can be made by the computer system within corresponding permissible degrees of movement that are defined by the orthodontics rules for the respective tooth type.

As an illustrative example, the computer system can use 2 datums on each incisor to level the respective incisor within the permissible degrees of movement for the incisor. The computer system can rotate the incisor so that it is level to the occlusal plane. The computer system can move the incisor until a tip of the incisor is level with the occlusal plane. Typically, the computer system may tip the incisor but may not torque the incisor. The computer system can perform a similar process for leveling the posterior teeth with the occlusal plane. For example, the computer system can select one tooth at a time and rotate any of the posterior teeth from a facial view of the model such that one or more marginal ridges of the posterior teeth are level with the occlusal plane and/or come into contact with the occlusal plane. In some implementations, the computer system can level each tooth independently of other teeth to the occlusal plane by identifying a pivot point for the tooth.

Optionally, the computer system can torque (e.g., move inward and outward) at least one tooth based on the tooth's angle relative to the occlusal plane. To torque the tooth, the computer system can use buccal and/or distal cusp tip datums so that the cusp tips of the tooth are parallel to the occlusal plane (e.g., so that cusp tips of a molar are parallel to the plane). After the tooth is torqued, the computer system may also tip the tooth. For example, the computer system can tip a posterior tooth using the marginal ridge datums for the tooth. Tipping a tooth can cause a root of the tooth to move mesially and/or distally from a facial view of the tooth. In some implementations, by default, the posterior teeth can be automatically torqued and tipped. In some implementations, the user can select whether they desire for the posterior teeth (and other teeth in the model) to be torqued, tipped, and/or rotated.

The computer system can snap the lower and/or upper teeth, individually and/or in sets/groups, to a same arch form within permissible degrees of snapping movement as defined by the orthodontics rules in block 328. Refer to FIG. 11 for further discussion. As described herein, the same arch form can be used for both upper and lower teeth. In orthodontics, the computer system can start with snapping the lower teeth to the lower arch form because there can be limited space for movement in the lower arch compared to the upper arch (e.g., the lower arch can have less bone in a jaw to work with than the upper arch, which can be defined by the orthodontics rules, as described herein). In some implementations, it does not matter whether the lower teeth are snapped to the lower arch form before snapping the upper teeth to the upper arch form or vice-versa. Sometimes, when teeth are snapped to the arch form, the teeth may overlap or otherwise collide. Adjustments can be made by the computer system at a later time to resolve any of these collisions. The purpose of snapping the teeth to the arch form is to ensure that the teeth are appropriately tipped and torqued to achieve a desired smile design while being within permissible degrees of movement as defined by the orthodontics rules.

The computer system can adjust all or a set of IP contacts in the digital model to remove tooth overlap and/or maintain relative tooth orientation (block 330). Such adjustments may also be made within permissible degrees of IP contact movement defined by the orthodontics rules. Refer to FIG. 12 for further discussion. For example, the computer system can solve for IP contacts by starting at a midline of anterior teeth and working back towards molars on each side of a respective arch form. Resolving the IP contacts can provide for resolving any interference problems between neighboring teeth, thereby enhancing the smile design for the patient and reducing overlap or overcrowding appearances of the patient's existing teeth. Resolving the IP contacts can be performed in 2D. Therefore, the previously determined tooth tips and/or torques may be locked in and the computer system may not tip and/or torque the tooth in block 330. A movement direction for resolving the IP contacts can be defined by centers of at least 2 neighboring teeth and a vector that passes through the centers of the neighboring teeth. The computer system can accordingly move each tooth along the vector in order to maintain a relationship between the neighboring teeth.

The computer system can optionally adjust vertical positioning of each tooth in the upper and/or lower arches in block 332. The vertical positioning movements can be made by the computer system so long as they fall within permissible degrees of vertical movement defined by the orthodontics rules and for the particular tooth type. Vertical adjustments can be made in orthodontic treatments, such as intruding a tooth by applying vertical pressure onto the tooth to cause the tooth to sink into the bone. Vertical adjustments can also be made such as applying negative pressure to the tooth, which can cause the tooth to pop out of a socket and for the socket to automatically fill in around the tooth and adjust its shape/position relative to the tooth. In some implementations, the orthodontics rules can prevent or otherwise not recommend moving teeth up and down (e.g., vertically) beyond predetermined permissible degrees of vertical movement when designing and selecting veneers. Refer to FIG. 13 for further discussion.

For example, the computer system can push/raise each lower tooth in a direction perpendicular to the occlusal plane until the lower tooth contacts the plane (block 334). Regarding the lower tooth, the computer system can keep moving the tooth until an occlusal point of the tooth reaches the occlusal plane (e.g., a point of each tooth that's closest to the occlusal plane gets pushed into contact with the occlusal plane when the occlusal plane is normal, such as when a Z coordinate of the occlusal plane is completely flat).

Additionally or alternatively, the computer system can sock each upper tooth until the upper tooth contacts one or more of the lower teeth (block 336). As described further in reference to FIG. 13, the computer system can push the upper teeth down until they come into contact with the lower teeth. Some teeth, such as canines of the upper teeth, may poke through the occlusal plane.

In block 338, the computer system can optionally iteratively adjust at least one upper tooth in, out, down, and/or up until a threshold distance for a predetermined overbite is achieved. The computer system can iteratively adjust each tooth independently of other teeth (e.g., perform the iterative adjustments on a tooth-by-tooth basis). The computer system can perform block 338 as part of performing one or more of the blocks 320-336. In some implementations, the computer system can perform block 338 after performing each of the blocks 320-336 and/or after performing any combination of the blocks 320-336. The threshold distance can vary based on the tooth/teeth being adjusted. For example, the threshold distance can be approximately 2 mm of overbite for central teeth and incisor teeth. As another example, the threshold distance can be approximately 1.5 mm for lateral teeth. The threshold distance can be set and/or determined by the computer system. In some implementations, the user can provide user input indicating one or more desired threshold distances, which can then be used by the computer system in block 338.

In block 338, the computer system can perform one or more iterative adjustments to nest and/or sock the upper posterior teeth. For example, for each upper posterior tooth, the computer system can move the tooth in and out, buccal-lingually, until a lowest point that the tooth can go is achieved (or some predetermined threshold distance is achieved). If, as an illustrative example, the tooth is moved in a buccal direction that causes the tooth to also move up, then the computer system can determine a distance from the tooth to one or more lower teeth (and/or the occlusal plane), divide the distance in half, and move the tooth in the opposite direction (e.g., lingually) by an amount the corresponds to half the distance. If the tooth is moved in the buccal direction and the tooth continues to move down, then the computer system can continue to move the tooth in the buccal direction until the desired lowest point/predetermined threshold distance is achieved.

The computer system can determine, in block 340, whether positioning of the teeth in the digital model satisfies the orthodontics rules and achieves a desired teeth arrangement for the patient. If the orthodontics rules are not achieved and the desired teeth arrangement/smile design is not achieved, the computer system can return to block 338 in the process 300 and iteratively adjust the teeth until the orthodontics rules are achieved and the desired teeth arrangement is achieved. For example, the computer system can iteratively adjust IP and/or vertical contacts between the upper and lower teeth until an amount of movement of the teeth satisfies a threshold amount of movement (e.g., approximately 10 microns of movement, or another threshold permissible degrees of movement defined by the orthodontics rules). In other words, iterative adjustments can be made to the upper and lower teeth until such adjustments cause no more than the threshold amount of movement to other teeth in the digital model.

If the orthodontics rules are satisfied and the desired teeth arrangement is achieved, the computer system can proceed to block 342, in which the computer system can anchor at least one tooth in the digital model. For example, the computer system can anchor molars in the model. Molars can be used as anchors, especially in orthodontics, since they are hard to move through bone. When performing an auto-setup process as described herein, teeth are set up from a midline back to the molars. While aligning teeth along an arch form, the teeth may shift, thereby causing the molars to move as well. Anchoring the molars can include distalizing the molars, which means pushing the molars forward into the mouth in their original positions. Distalizing the molars also may cause the entire arch form to slide forward so that the molars are returned to their original positions. As a result, upper anterior teeth may, for example, be pushed forward, thereby causing a more significant overbite than desired for the patient. This overbite can be corrected automatically by the computer system and/or manually by the relevant user. The user can view the overbite once the molars are anchored and determine appropriate treatment options to correct the overbite. As an illustrative example, the user and/or the computer system can perform interproximal reduction, in which some of the teeth are shaved off to allow for the molars to remain in their original positions while reducing the overbite. As another illustrative example, the user and/or the computer system can remove one or more teeth, such as first or second bicuspids, to maintain the molars in their original positions while reducing the overbite.

The computer system can then select veneers, caps, or other orthodontics dental appliances based at least in part on the position of the teeth in the digital model and the desired teeth arrangement for the patient (block 344). The veneers can be selected based on comparing tooth shapes, widths, and/or lengths to corresponding characteristics of the teeth in the digital model and/or the desired teeth arrangement for the patient. For example, a model can be trained to identify different tooth shapes in patient image data, then output the identified tooth shapes and/or confidence values/scores for those tooth shapes. The computer system can then identify and select veneers that have most similar or same tooth shapes and/or confidence values/scores. In some implementations, the models described herein can be trained to output a selected tooth library based on the determined measurements and/or shapes for the patient. A model can be trained to measure widths of the patient's teeth, then output the measured widths and/or selection of one or more veneers having same or similar tooth widths. A model can be trained to measure lengths of the patient's teeth, then output the measured lengths and/or selection of one or more veneers having same or similar tooth lengths. The computer system can use any combination of tooth shape, width, and/or length to select veneers to achieve the desired teeth arrangement for the patient. Refer to U.S. patent application Ser. No. 18/328,461 entitled “Systems and Methods for Library-Based Tooth Selection in Digital Dental Appliance Design” and U.S. patent application Ser. No. 18/328,584 entitled “Auto-Denture Design Setup Systems”, both of which are incorporated by reference in their entirety, for further discussion.

In some implementations, the computer system can process the image data of the patient to determine measurements of the patient's face and triangulation of the patient's facial features. The computer system can identify the patient's eye sockets and measure, for example, a distance between the eye sockets. This measurement can then be used to determine an appropriate size of veneers so that, when installed in the patient's mouth, the veneers appear natural and fitting for the patient's facial proportions. The computer system can implement a machine learning model to measure the patient's facial features from the image data, score sets of veneers as potential candidates for matching the patient's facial feature measurements, and/or select a set of veneers that best fits the patient's facial features (e.g., by selecting a set of veneers having a highest score and/or a score greater than a threshold score value).

The computer system can use one or more artificial intelligence (AI) algorithms to process the 2D image data of the patient's face, recognize lines, triangulate facial features, identify facial features and/or shape of the patient's face more generally, and/or identify and match a set of veneers with the patient's face.

The computer system can access, as another illustrative example, a table stored in the data store that includes correlations of veneer sizes/measurements to different sized/shaped facial features of patients. This lookup table can be used by the computer system to select a set of veneers that likely best matches the patient's facial features.

The computer system can also use the determined teeth movements and positioning from blocks 320-338 as indicators for determining which veneers may fit in the patient's mouth and/or comfortably overlay and adhere to the patient's existing teeth.

In block 346, the computer system can overlay the digital model with the selected veneers, caps, or other orthodontic dental appliances. For example, the computer system can position the selected veneers over the existing teeth in the model that have been adjusted and moved as described in reference to blocks 320-338. This can help the relevant user visualize how the veneers would look inside the patient's mouth, how the veneers would be fitted into and adhered onto the patient's existing teeth, and provide a comparison of the patient's existing teeth to the patient's mouth once their teeth are moved accordingly and the veneers are applied to their teeth.

Optionally, in block 348, the computer system may iteratively adjust one or more teeth in the digital model based on the orthodontics rule until the desired teeth arrangement is achieved. Once the veneers overlay the digital model, the computer system can identify that the veneers do not fit as preferred on top of the moved teeth. Therefore, the computer system can perform one or more iterative adjustments like the adjustments described in reference to blocks 320-338 to determine whether and how one or more of the teeth can be moved differently in order for the veneers to fit according to the desired teeth arrangement for the patient. In some implementations, the computer system can select and try one or more other sets of veneers on top of the arranged teeth in the digital model in block 348. As a result, the computer system can identify one or more other sets of veneers that may provide a better fit and/or appearance to achieve the desired teeth arrangement for the patient.

The computer system can augment the image of the patient based on the digital model in block 350. Block 350 can be performed once the computer system determines that the set of veneers and the teeth movements achieve the desired teeth arrangement for the patient. Augmenting the image can include processing the image to place the veneers of the digital orthodontics model over the patient's teeth in the image. Augmenting the image can also include adjusting a shape, size, and/or overall appearance of the patient's smile and other facial features based on the placement of the veneers and the teeth movements for the patient. The resulting augmented image can accurately and realistically depict what the patient's smile may look like when their teeth are moved according to the digital model and the selected set of veneers are applied to their moved teeth.

In block 352, the computer system can return the augmented image, tooth movement data, the digital model, and/or an orthodontics treatment plan as output. For example, the output can include images of the patient's smile with the veneers as they would fit in the patient's mouth. The output can include the digital model of the patient's mouth with the veneers. The output can include types of veneers for the patient, including but not limited to selection, shape, instructions about placement for the veneers. The output can include orthodontics instructions for moving the teeth in the patient's mouth, including directions of movement, types of movement, degrees of movement, recommended orthodontic dental appliances for moving the teeth, length of time for moving the teeth, etc.

The output can be transmitted to the user device and presented in a GUI at the user device. The output can be stored in the data store and retrieved at a later time for additional processing, analysis, and/or presentation. The output can be transmitted to other relevant computing systems, such as an orthodontics planning system. Refer to FIGS. 1A, 1B, and 1C for further discussion about returning the output and how the returned output can be used to help the patient achieve their desired teeth arrangement.

In some implementations, as described herein, the computer system can receive user input from the user device indicating one or more adjustments/modifications to teeth movements/positioning, veneers selection, orthodontics treatment plan, etc. The computer system can automatically adjust the digital model based at least in part on the user input.

FIGS. 4A and 4B are a flowchart of another process 400 for computer-automated smile design for a patient. Similar to operations described in reference to FIG. 1B, the process 400 can include operations for selecting veneers and then determining whether and how much the patient's existing teeth can be moved to achieve a desired teeth arrangement for the patient with the selected veneers.

The process 400 can be performed by the digital smile design computer system 102 described in reference to at least FIG. 1A. The process 400 can also be performed by any other type of computer system, computing device, cloud-based system, and/or network of computing systems. For illustrative purposes, the process 400 is described from the perspective of a computer system.

Referring to the process 400 in both FIGS. 4A and 4B, the computer system can receive patient scan data of the patient's existing orthodontics and an image of the patient (block 402). Refer to at least FIG. 1A for further discussion.

In block 404, the computer system can generate a digital orthodontics model for the patient based at least in part on the patient scan data. Refer to FIGS. 1A and 2 for further discussion.

The computer system can define an arch form in block 406. Refer to FIG. 1A and blocks 304-306 in FIG. 3A for further discussion.

The computer system can select a set of veneers from a plurality of sets of veneers to achieve a desired teeth arrangement for the patient (block 408). Refer to FIGS. 1A, 1B, 1C, and block 344 in FIG. 3C for further discussion about selecting the set of veneers.

The computer system can then overlay the digital orthodontics model with the selected set of veneers in block 410. Refer to at least FIGS. 1A, 1B, and block 346 in FIG. 3C for further discussion.

In block 412, the computer system can iteratively adjust one or more teeth in the model to achieve the desired teeth arrangement for the patient. Refer to at least block 348 in FIG. 3C for further discussion.

For example, the computer system can rotate, tip, and/or torque each tooth or one or more teeth (block 414). The computer system can snap one or more of the teeth to the arch form (block 416). The computer system can adjust relative positioning of adjacent teeth to resolve for collision (block 418). The computer system can optionally adjust vertical positioning of one or more teeth to correct for overbite (block 420). Refer to at least blocks 320-336 in FIG. 3B, and FIGS. 10A, 10B, 11, 12, and 13 for further discussion.

In some implementations, the computer system can adjust all the patient's teeth to achieve a desired orthodontics treatment plan for the patient. Sometimes, the computer system may adjust only some of the patient's teeth. For example, the computer system can determine which of the patient's teeth do not receive or fit well with the selected set of veneers. The computer system can then determine movements for those teeth of the patient. The computer system may also determine how movement of those teeth may cause one or more adjacent teeth to also be moved and adjusted. As an illustrative example, the computer system may adjust only the patient's upper anterior teeth to better fit placement of the selected set of veneers but may not adjust upper molars and/or a lower arch of teeth (e.g., where the selected set of veneers may fit as expected or within an expected threshold range for fit and placement, where veneers will not be fitted/installed).

In block 422, the computer system can retrieve orthodontics rules indicating a plurality of permissible degrees of movement for each tooth. Refer to FIG. 1A for further discussion. In some implementations, the computer system can retrieve only the orthodontics rules that correspond to the teeth that have been moved and/or the types of tooth movements that have been made. For example, if the computer system only torques incisors, the computer system can retrieve orthodontics rules corresponding to torque movements for incisors.

The computer system can determine, for each tooth adjustment, whether the adjustment satisfies corresponding permissible degrees of movement for the tooth (block 424). In contrast to the process 300 in FIGS. 3A, 3B, and 3C, in the process 400, the computer system can select the veneers and then determine whether the patient's teeth can be moved enough to achieve the desired teeth arrangement with the selected veneers, all while the teeth movements are acceptable according to the orthodontics rules. As described herein, the teeth movements and determination of block 424 can be iteratively performed until the desired teeth arrangement is achieved for the patient.

If the permissible degrees of movement are not satisfied (e.g., the tooth is not moved enough to achieve the desired tooth arrangement or the tooth is moved too much to fall outside a range of the permissible degrees of movement), then the computer system can identify which of the tooth adjustments does not satisfy the corresponding permissible degrees of movement for the respective tooth (block 434). The computer system can then return to block 412 and iteratively adjust the tooth corresponding to the identified tooth adjustment until the adjustment satisfies the permissible degrees of movement in block 424.

If the permissible degrees of movement are satisfied (e.g., the tooth is moved enough to achieve the desired tooth arrangement and the tooth is moved within the range of the permissible degrees of movement) in block 424, then the computer system proceeds to block 426, in which the computer system can generate teeth movement data. Generating teeth movement data can include generating instructions or steps to be used or followed by a relevant user in an orthodontics treatment plan for the patient. For example, the teeth movement data can indicate how many degrees of movement can and should be applied to each particular tooth in the patient's mouth to achieve the patient's desired teeth arrangement. The data can also indicate which teeth should be moved. In some implementations, the data can indicate what orthodontics treatment plans can be applied to the patient in order to achieve the teeth movements determined in the process 400 (e.g., whether the patient should use aligners, braces, retainers, etc.).

The computer system can also augment the image of the patient with the adjusted teeth and the veneers from the digital orthodontics model (block 428). Refer to FIG. 1A and block 350 in FIG. 3C for further discussion. In some implementations, block 428 can be performed before block 426. Sometimes, blocks 428 and 426 can be performed simultaneously/at a same time.

Optionally, the computer system can generate an orthodontics treatment plan in block 430. Block 430 can be performed as part of performing block 426. Refer to FIG. 1A and block 352 in FIG. 3C for further discussion.

In block 432, the computer system can return at least one of the teeth movement data, the augmented image, the orthodontics, treatment plan, the selected veneers, and/or the digital orthodontics model with the adjusted teeth and the veneers. Refer to FIG. 1A and block 352 in FIG. 3C for further discussion.

FIG. 5 is a flowchart of a process 500 for computer-automated selection of veneers for smile design without requiring movement of a patient's teeth. Unlike the processes 300 and 400 in FIGS. 3A, 3B, 3C, and 4A and 4B, the process 500 can be performed to select a set of veneers that best fit the patient's existing mouth and teeth placement so that the patient's teeth do not need to be moved. An orthodontics treatment plan may not be determined nor used for the patient.

The process 500 can be performed by the digital smile design computer system 102 described in reference to at least FIG. 1A. The process 500 can also be performed by any other type of computer system, computing device, cloud-based system, and/or network of computing systems. For illustrative purposes, the process 500 is described from the perspective of a computer system.

Referring to the process 500, the computer system can receive patient scan data of a patient's existing teeth and an image of the patient (block 502). Refer to block 402 in the process 400 of FIG. 4A for further discussion.

In block 504, the computer system can generate a digital dental model based at least in part on the patient scan data. Refer to block 404 in FIG. 4A for further discussion.

In block 506, the computer system can define an arch form. Refer to blocks 304-310 in the process 300 of FIG. 3A for further discussion.

The computer system can select a set of veneers from a plurality of sets of veneers to achieve a desired teeth arrangement for the patient (block 508). As described herein, the computer system can retrieve at least one set of veneers (e.g., a tooth library) from a data store. The computer system can retrieve/select the set of veneers that satisfy one or more selection criteria. For example, the computer system can select the set of veneers that includes metadata or other information indicating what patients can use the particular set (e.g., children, elderly people, various age groups, gender, etc.). The computer system can process the digital dental model to determine features about the patient's existing teeth, such as tooth measurements (e.g., width, length) and/or tooth shape. The determined features can then be compared to similar features identified for the plurality of sets of veneers to select the set of veneers having the most similar features, same features, or features within a threshold range of values to the determined features about the patient's existing teeth.

In some implementations, the computer system can select a first set of veneers from the plurality of sets for an upper arch form of the patient and a second set of veneers from the plurality of sets for a lower arch form of the patient. In yet some implementations, the computer system can select a set of veneers from the plurality of sets that includes teeth for both upper and lower arch forms, or a subset or portion of one or more of the upper and lower arch forms. For example, the computers system can select a first set of veneers that includes teeth for anterior teeth of both the upper and lower arch forms. The computer system can also select a second set of veneers that includes teeth for posterior teeth of the upper arch form. The computer system can select a third set of veneers that includes teeth for posterior teeth of the lower arch form. In some implementations, the computer system may only select sets of veneers for portions of the patient's teeth. For example, the computer system may only select at least one set of veneers to be installed on the patient's anterior teeth of the upper arch form.

As an example for selecting the set of veneers in block 508, the computer system can process the received data to identify a shape of the patient's existing teeth in block 510. Although identifying and selected veneers based on teeth shape is described in the context of the process 500, the computer system can also identify teeth shape as part of selecting veneers in any other of the processes described herein, such as the process 300 in FIGS. 3A, 3B, and 3C, and the process 400 in FIGS. 4A and 4B.

To process the data and identify the shape of the patient's existing teeth, the computer system can apply a machine learning model to the patient scan data, the image of the patient's teeth, and/or the digital dental model. The model can be trained to identify and classify different shapes of teeth for different types of teeth in different types of data (e.g., 3D data such as the patient scan data and/or the digital dental model, and 2D data, such as the image of the patient's teeth).

Training the model can include feeding the model with training data that includes 2D and 3D images of various different teeth. The 2D and 3D images can be labeled and/or annotated (e.g., by the computer system automatically, manually by relevant users such as dentists and technicians) with different markers, datums, or other identifiers on the teeth. The 2D and 3D images can be labeled and/or annotated with various types of teeth measurements. The 2D and 3D images can be labeled and/or annotated with various types of tooth shapes. The model can then be trained to identify the various markers, datums, identifiers, and/or measurements, which can then be used by the model to classify each tooth in 2D and 3D data as a particular shape. The model can also be trained to assign confidence values, scores, or other values (e.g., integers, numeric, Boolean, string) to each classification indicating a likelihood that a particular tooth has the shape that the model identified.

The model can be iteratively trained over time as the model makes runtime shape classifications and/or a relevant user overrides model decisions or provides input indicated other shape information than that determined and provided as output by the model. In some implementations, a model can be trained to identify upper arch teeth shapes and another model can be trained to identify lower arch teeth shapes. A model can be trained to identify all teeth shapes. Various models can also be generated and trained, where each model can be trained to identify a different type of teeth shape and/or teeth shapes for particular groups or sets of teeth.

The computer system can score each set of the plurality of sets of veneers (or a selected subset of the plurality of sets of veneers) based on a likelihood that the set achieves the desired teeth arrangement, has a similar shape to the shape of the patient's existing teeth, and/or is shaped to be received by the patient's teeth (e.g., curvature and/or size of the veneer teeth allow the veneer teeth to be attached comfortably, securely, safely, and/or correctly to the patient's existing teeth (block 512). The score can be a numeric value (e.g., integer) on a predetermined scale. The score can be assigned on a scale of 0 to 5, 0 to 10, 0 to 100, 1 to 5, 1 to 10, 1 to 100, etc. The score can also be assigned string and/or Boolean values. A higher score can indicate a higher likelihood of matching shape to achieve a desired teeth arrangement for the patient. A lower score can indicate a lesser likelihood of matching shape to achieve a desired teeth arrangement for the patient.

As an illustrative example, the computer system can select a subset of the plurality of sets of veneers having shape information that matches or is most similar to the identified shapes of the patient's existing teeth. The computer system can then score each of the sets of veneers in the subset based on similarity of the shape information for the set with the identified shapes of the patient's existing teeth. In some implementations, the computer system can score each set of the plurality of sets of veneers, instead of first selecting the subset.

The computer system can score the sets of veneers on an individual tooth basis, an overall arch form, and/or sets of teeth. For example, the computer system can score each tooth in a set of veneers based on the tooth's similarity in shape to a corresponding tooth shape for the patient (e.g., compare an upper arch left incisor in the set of veneers to an upper arch left incisor in the patient's teeth). As another example, the computer system can score the shape(s) of an entire arch of teeth in the set of veneers (e.g., an upper arch form) relative to a corresponding arch form's shape(s) for the patient. As yet another example, the computer system can score the shape(s) of a set of teeth in the set of veneers (e.g., upper arch anterior teeth, upper arch posterior teeth, upper arch molars, upper arch incisors, lower arch anterior teeth, lower arch posterior teeth, lower arch molars, lower arch incisors, etc.) in comparison to a corresponding set of teeth for the patient.

Sometimes, the computer system can score each tooth individually based on shape, then aggregate scores for at least a subset of the teeth in the set of veneers. For example, the computer system can score each individual tooth's shape, then aggregate the scores for all the upper arch teeth into a first score and aggregate the scores for all the lower arch teeth into a second score. As another example, the computer system can score each individual tooth's shape, then aggregate the scores for all upper anterior teeth into a first score and aggregate the scores for all lower anterior teeth into a second score. In some implementations, the computer system can determine and/or combine scores for both upper and lower arch teeth into an aggregate score. This can be beneficial in scenarios in which the computer system selects a set of veneers that applies to both upper and lower arches. In some implementations, the computer system can select and score sets of veneers for the upper arch teeth separately from selecting and scoring sets of veneers for the lower arch teeth (in such scenarios, the computer system may also select sets of veneers separately for the upper and lower arch forms).

The computer system can select a set of veneers having a highest score or a score that exceeds a threshold score value in block 514. In the example where the computer system selects the subset of sets, the computer system can score each set in the subset, then select the set having the highest score. The computer system can determine multiple scores for each set of veneers and then select the set amongst the plurality of sets of veneers having a particular score that is the highest. For example, the computer system can select a set of veneers having a highest score for upper arch anterior teeth, then select a different set of veneers having a highest score for lower arch anterior teeth. The computer system can aggregate any combination of scores for each set of veneers and then select the set of veneers having the highest score.

Sometimes, the computer system can select one or more sets of veneers having respective score(s) that exceed(s) the threshold score value (e.g., top 3 scoring sets of veneers). The computer system can then present the one or more sets of veneers to the relevant user at their user device and the user can select which of the sets of veneers to try for the patient (e.g., trying a set of veneers can include presenting the set of veneers as overlaying the digital dental model for the patient in a GUI at the user device).

As described above, the model that identifies shapes of teeth in the sets of veneers can also determine confidence values for each identified shape. The computer system can use the confidence values as the scores for each set of veneers and then select the set of veneers in block 514 that has a highest confidence value for a particular tooth shape and/or a highest aggregate confidence value for one or more tooth shapes.

In block 516, the computer system can overlay the digital model with the selected set of veneers using the defined arch form. The computer system can align the teeth of the selected veneers with the patient's existing teeth in the model. By overlaying the model with the teeth of the selected veneers, the computer system can visually depict how the set of veneers may look for the patient. The model with the overlaid teeth of the selected veneers can be presented in a GUI at the user device, as described herein.

In some implementations, the computer system can overlay the digital model with the teeth of the selected veneers as part of selecting the set of veneers in blocks 508-514. For example, the computer system can overlay the model with each set of veneers, then score the overlaid set of veneers based on how well the veneers fit with the patient's existing teeth in the model. The computer system can use one or more criteria to determine whether the veneers fit with the patient's existing teeth, as described further below in reference to block 518. The one or more criteria can be used by the computer system to score the veneers fit. The computer system can then iterate through overlaying the digital model with each set of veneers to score the respective set. As another example, the computer system can score each set of veneers as described in reference to block 512. As part of block 514, selecting the set having the highest score, the computer system can overlay the digital model with each set of veneers having respective scores that exceed the threshold score value, and then apply the one or more criteria to determine which of the overlaid sets of veneers has the best fit and/or appearance with the patient's existing teeth in the model.

Once the model is overlaid with the teeth of the selected set of veneers, the computer system can determine whether the selected set of veneers fits the patient's existing teeth to not require any teeth movements (block 518). The computer system can retrieve one or more criteria, such as rules, from the data store described herein. The computer system can then use the rules to determine whether the selected set of veneers fits the patient's existing teeth and thus would not require the patient's teeth to be moved. Sometimes, the computer system can determine that a portion of the selected set of veneers fits some of the patient's teeth but not others. The computer system can return information, in such scenarios, indicating that the portion of the selected set of veneers should be used but that another set of veneers should be tested with the digital dental model for fitting the other portion(s) of the patient's teeth.

The one or more criteria or rules that are used in block 518 can be used by the computer system to check proportions and appearance of the selected set of veneers with the patient's facial features. For example, the computer system can process the image data of the patient to identify locations of the patient's eyes and/or shapes of different facial features, as described herein. The computer system can use triangulation techniques and/or other processing techniques to determine proportions of the patient's face and an appropriate size for the patient's smile and/or teeth relative to their proportions. Then, the computer system can determine whether the selected set of veneers, when overlaying the model of the patient's existing teeth, would fit with one or more of the proportions and/or shapes of the patient's face.

The one or more criteria or rules can additionally or alternatively be used by the computer system to check whether the veneers fit to a curve, shape, and/or size of the patient's existing teeth and/or mouth (e.g., when the patient smiles, which can be depicted in the image data of the patient). The one or more criteria or rules can additionally or alternatively be used by the computer system to determine whether the selected set of veneers fits, complies with, or otherwise matches the desired teeth arrangement for the patient.

If the selected set of veneers would require teeth movements (e.g., a minimum threshold amount of movement, any amount of movement), the computer system can select another set of veneers from the plurality of sets of veneers to test to achieve the desired teeth arrangement for the patient (block 520). To select another set, the computer system can select the set with the next highest score (e.g., next highest individual score, next highest aggregate score). The computer system can then proceed to block 516 and repeat blocks 516-520 until the desired teeth arrangement is achieved with veneers that would not require moving the patient's teeth.

If the selected set of veneers fits the patient's existing teeth without requiring moving the patient's teeth in block 518, the computer system can proceed to block 428 in the process 400 in FIG. 4B and perform blocks 428 and 432 (e.g., augment the patient image data and return output).

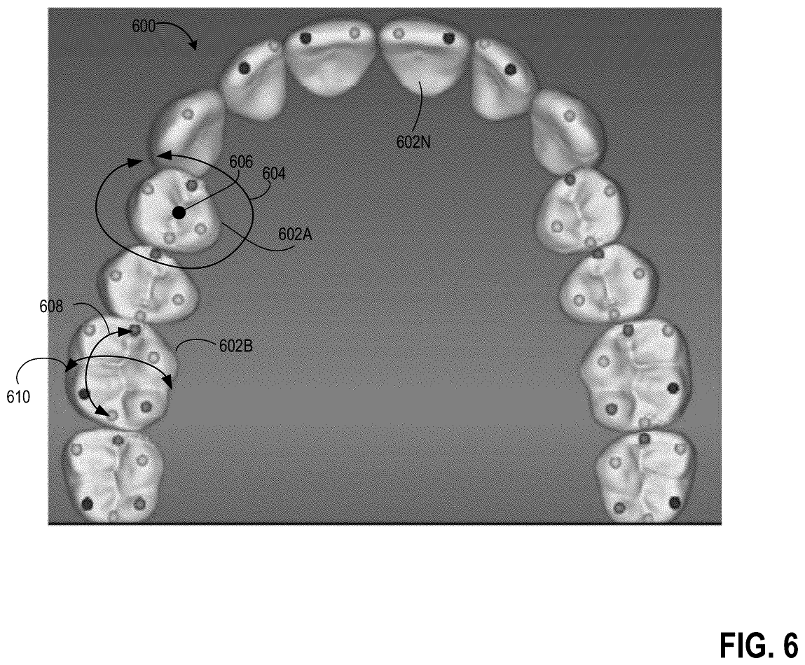

FIG. 6 illustrates example adjustments of rotating 604, tipping 608, and/or torqueing 610 one or more teeth 602A-N in a digital orthodontics model 600. The rotating 604, tipping 608, and/or torqueing 610 can be performed automatically by the computer system described herein and/or manually by a relevant user at their respective user device. Refer to blocks 320-326 in the process 300 in FIG. 3B for further discussion. Although rotating, tipping, and torqueing are described and depicted in reference to lower teeth in the model 600, the same or similar techniques can also be applied to upper teeth in the model 600.

In the example of FIG. 6, a tooth 602A is rotated 604 around a pivot point 606. The pivot point 606 can be defined as a midpoint between 2 datums of the tooth 602A. The pivot point can be identified as a valley between one or more marginal ridges in the tooth 602A, in some implementations.

Tooth 602B can be tipped 608 and/or torqued 610. When the tooth 602B is tipped, the tooth 602B can be rotated laterally (e.g., around a pivot point) when viewing the tooth 602B from a front facing view. In some implementations, the tooth 602B can be tipped relative to its contact points with one or more adjacent teeth in the model 600. In some implementations, only anterior teeth in the model 600, such as the tooth 602N, may be tipped.

When the tooth 602B is torqued, a root of the tooth 602B can be rotated in and out (e.g., in and out of a mouth, or buccal-lingually). The torque adjustments can impact how far embedded the root of the tooth 602B is inside the patient. Posterior teeth in the model 600, such as molars, can be torqued. In some implementations, one or more of the anterior teeth, such as the tooth 602N, can be torqued.

The computer system can move any of the teeth 602A-N as described herein so long as the movements are within predefined permissible degrees of movement defined by orthodontics rules. Sometimes, the orthodontics rules can indicate that one or more of the teeth 602A-N cannot be moved or adjusted in certain directions, types of movements, and/or degrees.

In scenarios where the user selects one of the teeth 602A-N in a GUI at their user device to move the tooth, the user may move the selected tooth until they are unable to move the tooth anymore. For example, the computer system can visually limit the amount of movement available so that when the user clicks and drags the tooth in a particular direction and the movement limit is reached, the tooth will not move more in that particular direction, even if the user continues to click and drag on the tooth. Thus, the user can see just how far they can move the tooth before movement is no longer accepted/permissible. As described herein, the permissible movements can be limited to a particular type of tooth, a particular type of treatment plan, and/or patient-related information (e.g., a shape of the patient's mouth, an amount of space in the patient's jaw for teeth movement, etc.).

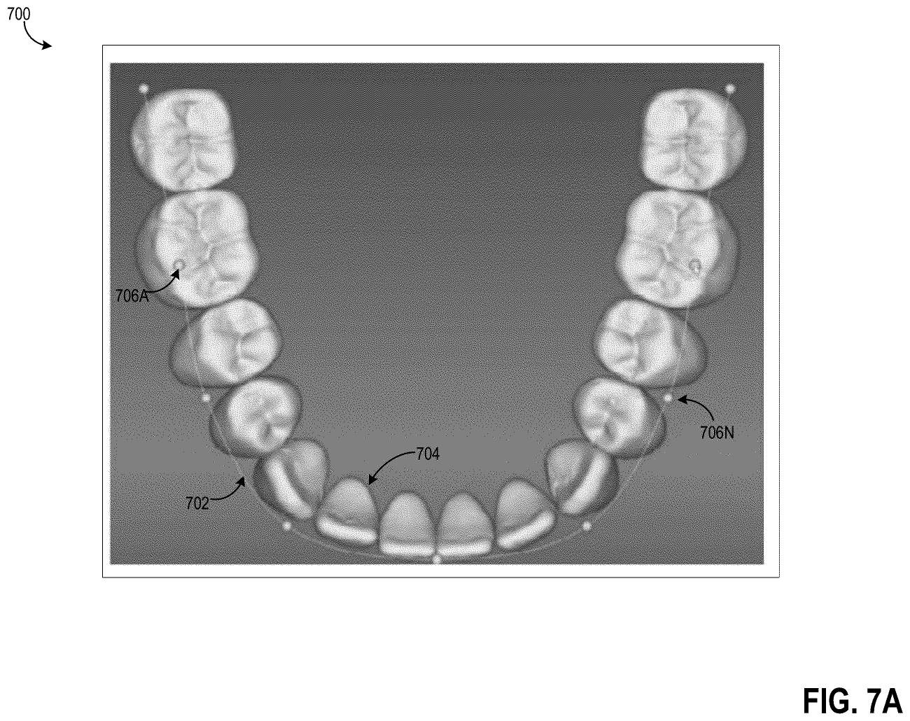

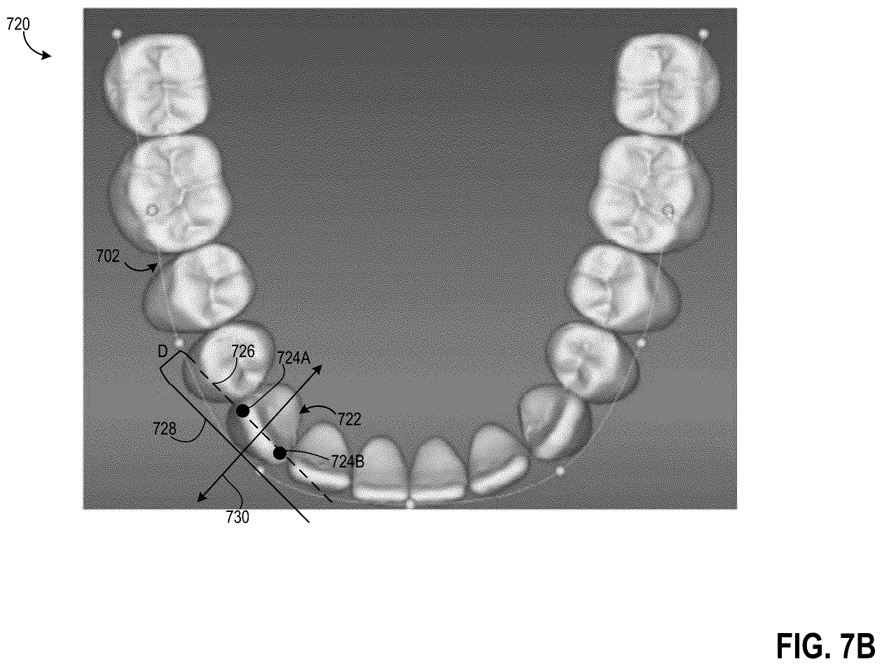

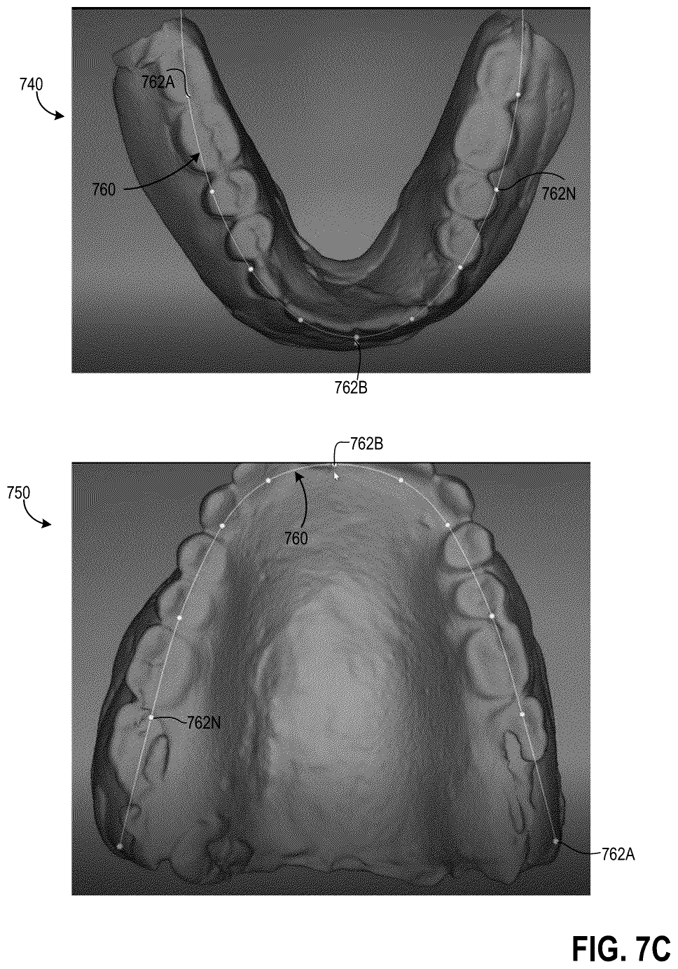

FIG. 7A is an example GUI 700 that may be generated by the digital smile design system described herein for identifying an arch 702 of a digital orthodontics model 704. The GUI 700 shows the example arch curve 702, which can be used for adjusting the patient's existing teeth for an orthodontics treatment plan. The arch 702 can also be used to overlay sets of veneers in the model 704 and select a best fitting or preferred set of veneers to achieve a desired teeth arrangement for a particular patient. As described herein, the arch 702 may be used to initially position teeth in the digital orthodontics model 704 and/or move the patient's teeth to achieve the desired teeth arrangement for the patient.

Here, the arch curve 702 is a spline curve shown with control points 706A-N. The control points 706A-N can be shown as spheres. The control points 706A-N can also be visually depicted in any other variety of ways. In some implementations, the GUI 700 accepts inputs from a user to change positions of one or more of the control points 706A-N (e.g., a drag may reposition a selected control point) and causes the computer system described herein to adjust a shape of the arch 702 accordingly. In some implementations, the teeth in the model 704 can be repositioned as the arch 702 changes. In some implementations, the arch 702 may be adjusted independently of the teeth. The GUI 700 may be configured to accept one or more inputs (e.g., a button or menu actuation) to cause the teeth to re-align to the arch 702. In some implementations, adjusting control points 706A-N on one side of the arch 702 can cause the computer system described herein to mimic the control point adjustments on an opposite side of the arch 702 to provide a uniform arch form for the model 704. In some implementations, adjustments can be made to only one side of the arch 702.

FIG. 7B illustrates snapping one or more lower teeth to the arch 702 of a digital orthodontics model. In the example of FIG. 7B, lower teeth 720 are shown with the arch 702 overlaying a portion of the lower teeth 720. A lower tooth 722 of the lower teeth 720 can be snapped to the arch 702 by the computer system using the disclosed techniques. For example, 2 points 724A and 724B on the tooth 722 can be identified by the computer system. The points 724A and 724B can be datums that were previously identified by the computer system, as described at least in reference to the process 300 in FIGS. 3A, 3B, and 3C. The points 724A and 724B can be opposing cusp tips or edges on the tooth 722. A reference line 726 can be identified to pass through the points 724A and 724B of the tooth 722. The tooth 722 can then be snapped by the computer system by rotating the tooth 722 until the reference line 726 is parallel with a tangent line 728. The line 728 can be tangent to a curve of the arch 702. Additionally or alternatively, snapping the tooth 722 to the arch 702 can include moving the tooth 722 in and out (e.g., buccal-lingually) in a direction illustrated by an arrow 730. The tooth 722 can be moved in and out until a desired distance D is achieved between the reference line 726 and the tangent line 728.

The operations described herein to snap the tooth 722 to the arch 702 can also be performed to snap the other teeth in the digital model to the arch 702. In other words, the computer system can snap each tooth to the arch 702 on an individual, tooth-by-tooth basis. In some implementations, the computer system can snap a group or set of teeth to the arch 702 at a time. The computer system can also snap upper teeth separately from the lower teeth 720. For example, the computer system can snap the lower teeth 720 by moving the lower teeth 720 to be on, or otherwise have a buccal or facial surface of the lower teeth 720, touching the tangent line 728. The computer system can snap the upper teeth by moving the upper teeth to be positioned a predetermined distance beyond the tangent line 728. In other words, the upper teeth can be positioned so that a buccal or facial surface of the upper teeth pass the tangent line 728 and are positioned closer to the patient's cheek rather than the patient's tongue. Such snapping adjustments can be made to achieve a desired overbite for the patient. As described herein, any of the snapping adjustments that are made to the teeth along the arch 702 can be made within predefined permissible degrees of movement defined by orthodontics rules.

FIG. 7C illustrates an arch form 760 for both upper and lower teeth 750 and 740, respectively, of a digital orthodontics model. As described herein, the same arch form 760 can be used for both the upper teeth 750 and the lower teeth 740. A user, as described herein, can click, select, and/or drag any points 762A-N to adjust the arch form 760. When the user moves one of the points 762A-N in the arch form 760 of the lower teeth 740, a same adjustment can be replicated, by the computer system, in the arch form 760 of the upper teeth 750. In some implementations, the user can select an option to stop mirroring the arch form 760 for the upper and lower teeth 750 and 740. Therefore, adjustments made to the lower teeth 740 by moving the points 762A-N along the arch form 760 for the lower teeth 740 may not be replicated by the computer system for the upper teeth 750.

In some implementations, although the same arch form 760 can be used for the upper and lower teeth 750 and 740, the user can change a placement of the entire arch form 760 for one or both of the upper and lower teeth 750 and 740. For example, the computer system can define the arch form 760 for the lower teeth 740. The computer system can then replicate or mirror the arch form 760 for the upper teeth 750 and present the upper teeth 750 with the arch form 760 in a GUI at the user's device. The user may decide that the arch form 760 is closer to a lingual surface of some of the upper teeth 750 rather than a buccal surface of the upper teeth 750. Therefore, the user may select the arch form 760 for the upper teeth 750 and move the arch form 760 to better align with the buccal surface of the upper teeth 750. The computer system may not make this adjustment to the lower teeth 740, especially if the computer system had originally defined the arch form 760 for the lower teeth 740 in alignment with a buccal surface of the lower teeth 740.

As described herein, any adjustments made to the arch form 760 can be made within permissible degrees of movement as defined by orthodontics rules. In some implementations, permissible degrees of movement for the upper teeth 750 can be different than permissible degrees of movement for the lower teeth 740.

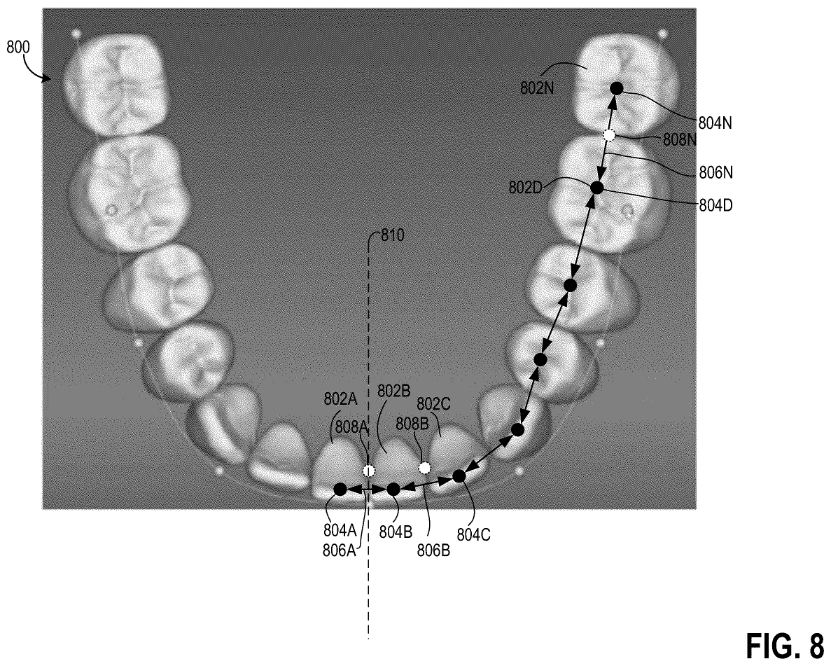

FIG. 8 illustrates adjusting teeth 802A-N in a digital orthodontics model within permissible degrees of movement to resolve IP contacts. The teeth 802A-N can be part of a lower arch 800, as shown in an occlusal view in FIG. 8. The computer system described herein can start by selecting a tooth at a midline 810 of the lower arch 800. The computer system can select the tooth 802A and work from the midline 810 to the last tooth 802N on one side of the lower arch 800. Once adjusting the teeth on the one side is complete, the computer system can return to the midline 810 and adjust the teeth back to a last tooth on the opposite side of the lower arch 800.

The computer system can resolve any interference or collisions between the teeth 802A-N. The tooth 802A can be a central tooth having a contact point 808A with the adjacent tooth 802B. The computer system can identify a center point 804A and 804B, respectively, for each of the tooth 802A and 802B. The computer system can then define a vector 806A that goes through the center points 804A and 804B. The computer system can resolve interference of the teeth 802A and 802B by moving one or both of the teeth 802A and 802B along the vector 806A. Movements along the vector 806A can also be limited by an amount of movement that is permissible for the teeth 802A and 802B, as defined by orthodontics rules.

The same operations can be performed to identify a contact point 808B between the tooth 802B and the tooth 802C, identify respective center points 804B and 804C, and define a vector 806B along which to move one or both of the teeth 802B and 802C to resolve any interference. A permissible range of movement along the vector 806B can also be defined by orthodontics rules for the particular teeth 802B and 802C. The same operations can be performed to identify a contact point 808N between the tooth 802D and the tooth 802N, identify respective center points 804D and 804N, and define a vector 806N along which to move one or both of the teeth 802D and 802N to resolve any interference, where a permissible range of movement along the vector 806N can be defined by orthodontics rules for the particular teeth 802D and 802N.

In some implementations, as described further in reference to FIG. 12, the computer system can first identify all center points 804A-N and all contact points 808A-N. The computer system can then define all vectors 806A-N. The computer system can retrieve orthodontics rules indicating permissible amounts of movement between each set of teeth along the vectors 806A-N. Then, the computer system can work tooth by tooth, from the midline 810 to the last tooth 802N on one side of the lower arch 800 to move the teeth 802A-N along the vectors 806A-N until the teeth 802A-N are in appropriate contact at the respective contact points 808A-N and so long as the movements fall within the permissible amounts of movement defined by the orthodontics rules.

FIG. 9 is a conceptual diagram of system components for selecting veneers. The digital smile design computer system 102 described herein can include a tooth library selection engine 2210, an optional auto-design denture setup engine 2200, a perfect smile setup engine 2202, and/or an optional auto-design implant setup engine 2206. The computer system 102 can include additional or fewer engines. The tooth library selection engine 2210 can communicate (e.g., wired and/or wirelessly via networks described herein) with one or more of the engines 2200, 2202, and 2206.

The perfect smile setup engine 2202 can transmit a request (block A, 900) to the tooth library selection engine 2210 for a subset of sets of veneers and/or caps to be used in designing a patient's smile.

Upon receiving the request, the tooth library selection engine 2210 can perform the techniques described herein to select the sets of veneers. The engine 2210 can select sets of veneers that can be overlaid with a digital orthodontics model of the patient to identify a best fitting set of veneers for the patient's face. As another example, the engine 2210 can select a set of veneers to be used for the patient's smile design. Refer to FIG. 1A and at least block 344 in the process 300 of FIG. 3C for further discussion.

The engine 2210 can then transmit the selected set or sets of veneers to the perfect smile setup engine 2202 (block B, 902). The engine 2202 can then auto-design the patient's smile using the selected set of veneers as described throughout this disclosure.

Similarly, the auto-design denture setup engine 2200 can transmit a request (block A, 900) to the tooth library selection engine 2210 for a subset of tooth libraries and/or at least one candidate tooth library to be used in designing dentures for a particular patient. The engine 2210 can use different selection criteria for the engine 2200 request versus the perfect smile setup engine 2202 request.

As another example, the auto-design implant setup engine 2206 can transmit a request (block A, 900) to the tooth library selection engine 2210 for a subset of tooth libraries and/or at least one candidate tooth library to be used in designing dental implants or inserts (e.g., crowns, caps) for a particular patient. The engine 2210 can use different selection criteria for the auto-design implant setup engine 2206 request versus the auto-design denture setup engine 2200 request and/or the perfect smile setup engine 2202 request. Designing implants and inserts may have smaller degrees of freedom regarding movement and position than designing dentures or even veneers because the implants or inserts have to fit into surface area of the patient's existing teeth and/or bone in the patient's upper and/or lower jaws. Refer to U.S. application Ser. No. 18/328,461 entitled “Systems and Methods for Library-Based Tooth Selection in Digital Dental Appliance Design,” filed on Jun. 2, 2023, the disclosure of which is incorporated by reference in its entirety, for further discussion.

FIGS. 10A and 10B are a flowchart of a process 1000 for automatically leveling teeth in a digital orthodontics model for auto-smile design. Any time that a tooth is tipped, torqued, and/or rotated, such changes can be checked against respective permissible degrees of movement that correspond to the tooth type. The permissible degrees of movement can be define so that the tooth may be moved up to a point before a root of the tooth loses connectivity and fails. As a result, the tooth may only be tipped, torqued, and/or rotated as safe, acceptable, and/or fitting for an orthodontics treatment plan. As described throughout this disclosure, the computer system can retrieve and use orthodontics rules identifying the permissible degrees of movement per tooth type and/or per movement type. As another example, the computer system can determine an offset between adjacent teeth and tip or otherwise move the teeth by the determined offset, then check the offset against the permissible degrees of movement defined by the orthodontics rules. The process 1000 can be used to determine how to move the patient's teeth for aligners, braces, or other types of orthodontic treatment plans, regardless of whether such orthodontic treatment plans include veneers.

The process 1000 can be performed by the digital smile design computer system 102 described in reference to at least FIG. 1A. The process 1000 can also be performed by any other type of computer system, computing device, cloud-based system, and/or network of computing systems. For illustrative purposes, the process 1000 is described from the perspective of a computer system.

Referring to the process 1000 in FIGS. 10A and 10B, the computer system can retrieve a digital orthodontics model with labeled datums on teeth for a patient and patient data (block 1002). The patient data can include, as described herein, images of the patient (e.g., an image of the patient smiling, 2D and/or 3D images of the patient's existing teeth). The patient data can include information about a desired teeth arrangement for the patient, such as how the patient would like their smile to look, dentist and/or technician notes about what the patient desires to have and/or what the dentist and/or technician believes can be the desired teeth arrangement for the patient. Refer to block 302 in the process 300 of FIG. 3A for further discussion.

The computer system can retrieve orthodontics rules indicating permissible degrees of movement for each tooth in block 1004. Refer to FIG. 1A and block 319 in the process 300 in FIG. 3A. In some implementations, the orthodontics rules can be retrieved throughout the process 1000, such as when the computer system is selecting and identifying a particular tooth to be moved and once the computer system moves the particular tooth. For example, the computer system can select a tooth to be leveled. The computer system can perform one or more leveling movements described herein on the tooth, then retrieve one or more orthodontics rules from a data store having permissible degrees of movement for moving that tooth. Using the retrieved rules, the computer system can determine whether the movements are appropriate for the tooth. If so, the computer system can move on to adjusting/leveling another tooth, and can repeat the operations of retrieving rules corresponding to the another tooth and comparing the rules to the adjustments made/leveling done to the another tooth.

In block 1006, the computer system can identify a plane defined by a plurality of datums on each anterior tooth in the digital orthodontics model. The computer system can define the plane as passing through 2 incisor tip edges or datums of a tooth. The plane can be used for leveling the tooth.

In block 1008, the computer system can identify an existing starting point for the anterior tooth. For example, the computer system can identify or otherwise determine measurements, angle, and/or degrees of a current position of the anterior tooth relative the plane. The starting point can be measured from a midpoint of the tooth (e.g., the computer system can identify a halfway point between 2 opposing datums on lateral edges of the tooth). The starting point can be measured from a tip of the tooth, which can be identified by at least one datum.

The computer system can then determine, based on the orthodontics rules, a permissible degree of movement to level the anterior tooth with an occlusal plane (block 1010). The computer system can measure an offset (e.g., in degrees, mm, microns) between the starting point of the anterior tooth and a maximum permissible movement or positioning of the tooth. As another example, the computer system can process the patient data to identify and measure a desired position of the particular tooth in the desired teeth arrangement. The computer system can then determine an offset or difference between the starting point of the tooth and the desired position of the tooth. The computer system can determine whether the determined offset or difference is within permissible degrees of movement for the tooth that are defined by the orthodontics rules. If so, then the computer system can proceed with operations described below, in which the computer system can make adjustments to the tooth based on the determined offset or difference.

In some illustrative examples, if the movement needed to adjust the teeth is not within the permissible degrees of movement for the tooth, the computer system can return a notification or instructions indicating that other treatment options can or should be pursued. The computer system can recommend surgery, as an example, in which bone grafting and/or other procedures can be performed to reposition bone in the patient's mouth. Once the bone is repositioned surgically, one or more orthodontics treatment plans can be applied to achieve the desired teeth arrangement for the patient. One or more teeth movements can also be made, followed by cosmetics touchups.

The computer system can tip the anterior tooth by the determined permissible degree of movement and according to the plane at a pivot point to level a tip of the anterior tooth with the occlusal plane (block 1012). The pivot point can be defined by the plane. The pivot point can be defined by at least one of the datums for the anterior tooth. In some implementations, the pivot point can be the midpoint or center point of the tooth and/or the plane that is defined by the datums of the tooth. The computer system can tip the anterior tooth until its tip, for example, is parallel with the occlusal plane. In some implementations, the anterior tooth can be tipped until the plane defined by the plurality of datums is parallel with an occlusal plane. Although tipping the anterior tooth is described here, the computer system can also perform one or more other movements of the tooth, such as rotating the tooth and/or torqueing the tooth. Sometimes, the computer system may be limited in performing one or more types of movements as defined by the orthodontics rules. As an illustrative example, the rules can indicate that for a patient that is 35 years old, their central incisors may be tipped no more than 4 mm before gum or jaw damage may result. For this patient, the computer system may only perform tipping movements to the patient's central incisors that max out at 4 mm of total movement from starting points of the central incisors.

The computer system can then determine whether there are more anterior teeth to level in block 1014. If there are more anterior teeth to level, the computer system can return to block 1008 and iterate through blocks 1008-1012 for each remaining tooth until there are no more teeth to level. If there are no more anterior teeth to level, the computer system can proceed to block 1016, described below.

Sometimes, the computer system may not adjust or level all the anterior teeth. The computer system can analyze the digital orthodontics model and determine which of the anterior teeth may not be straight, level, or otherwise in a permissible position to receive veneers (the permissible position can be defined by rules such as the orthodontics rules described herein). The computer system can then adjust only those teeth. In some implementations, the computer system can start the process 1000 with adjusting the patient's central incisors. Then the computer system can determine whether (i) any other anterior tooth are not straight, level, etc. and/or (ii) the adjustments to the central incisors cause collisions, interference, or other positioning issues with adjacent teeth. If (i) any other anterior tooth is not straight, level, etc. and/or (ii) the adjustments cause issues with adjacent teeth, the computer system can return to block 1008 for each of the anterior teeth to be adjusted.

In block 1016, the computer system can select a posterior tooth in the digital orthodontics model. The computer system can start, for example, with a posterior tooth adjacent one of the anterior teeth. The computer system may start with a posterior tooth near one of the anterior teeth that was adjusted as described above. In yet some implementations, the computer system can start with a posterior tooth that the computer system determines needs to or should be adjusted to achieve the desired teeth arrangement (regardless of adjustments that have or have not been made to the anterior teeth).

The computer system can identify a pivot point for the posterior tooth as a midpoint between 2 marginal ridge datums for the posterior tooth (block 1018). In some implementations, the pivot point can already be identified in the model. The computer system can simply select the pivot point in block 1018, as a result.

The computer system can identify an existing starting point for the posterior tooth in block 1020. Refer to block 1008 for further discussion about identifying the existing starting point for the particular tooth.

In block 1022, the computer system can determine, based on the orthodontics rules, a permissible degree of movement to level the posterior tooth with the occlusal plane. Refer to block 1010 for further discussion about determining the permissible degree(s) of movement for the particular tooth. As described throughout this disclosure, the computer system can retrieve different orthodontics rules for each type of tooth. The orthodontics rules can define different permissible degrees of movement and/or types of movement for each tooth. The computer system can determine an offset between the starting point for the posterior tooth and a maximum permissible position of the posterior tooth defined by the orthodontics rules. The computer system can determine an offset between the starting point for the posterior tooth and a desired position of the posterior tooth for the desired teeth arrangement. The offset can be the permissible degree(s) of movement determined in block 1022 for the particular tooth.

The computer system can rotate the posterior tooth by the permissible degree of movement and around a line perpendicular to the midpoint to level the posterior tooth with the occlusal plane (block 1024). Rotating the tooth around that perpendicular line can allow for the 2 or more marginal ridges to remain at a same distance away from the occlusal plane. Refer to block 1012 for further discussion about adjusting a position of the tooth based on and within the permissible degree of movement.

Optionally, the computer system may additionally or alternatively torque the posterior tooth by the determined permissible degree of movement and using buccal and/or distal cusp datums so that a cusp of the posterior tooth is parallel to the occlusal plane (block 1026). Similar to blocks 1022 and 1024, the computer system can determine a permissible degree of torqueing movement for the particular posterior tooth. The permissible degree of torqueing movement for the particular posterior tooth can be different than the permissible degree of rotating movement described in reference to blocks 1022 and 1024.

Optionally, the computer system may additionally or alternatively tip the posterior tooth by the determined permissible degree of movement mesially and/or distally using the 2 marginal ridge datums for the posterior tooth (block 1028). Similar to blocks 1022, 1024, and 1026, the computer system can determine a permissible degree of tipping movement for the particular posterior tooth. The permissible degree of tipping movement for the particular posterior tooth can be different than the permissible degree of rotating movement and the permissible degree of torqueing movement described above.

Once the computer system completes leveling the tooth, the computer system can determine whether there are more posterior teeth to level in block 1030. If there are more teeth to level, the computer system can return to block 1020 and iterate through blocks 1020-1028 until all the posterior teeth have been leveled.

As described above in reference to leveling the anterior teeth, the computer system may level all the posterior teeth or a portion thereof. For example, the computer system may only iterate through the blocks 1020-1028 for posterior teeth that are adjacent to adjusted anterior teeth. The computer system may iterate through the blocks 1020-1028 for posterior teeth that are adjacent to adjusted posterior teeth. The computer system may iterate through the blocks 1020-1028 for posterior teeth that appear to have collisions or interference with other adjusted teeth. The computer system may iterate through the blocks 1020-1028 for posterior teeth that are receiving veneers.

If there are no more posterior teeth to level, the computer system can proceed to block 1032, in which the computer system selects a set of veneers based on the patient data. Refer to FIG. 1A and at least block 344 in the process 300 of FIG. 3C for further discussion about selecting the set of veneers.

The computer system can overlay the leveled teeth in the digital orthodontics model with the selected set of veneers in block 1034. Refer to FIG. 1A and at least block 346 in the process 300 of FIG. 3C for further discussion.

The computer system can then return information indicating at least one of the selected set of veneers, the digital orthodontics model, teeth movement instructions, and a dental treatment plan (block 1036). Refer to FIG. 1A and at least block 352 in the process 300 of FIG. 3C for further discussion.

In some implementations, the process 1000 can be performed to move only some teeth. For example, the computer system may perform the process 1000 only for teeth that may receive veneers. The computer system may perform the process 1000 for teeth receiving the veneers so that the teeth are better aligned to receive the veneers in the desired teeth arrangement for the patient. The computer system may perform the process 1000 only for teeth that may not receive veneers. As an illustrative example, only upper anterior teeth may receive the veneers. The veneers may be fitted to current positioning of the patient's upper anterior teeth. Therefore, the upper anterior teeth may not be adjusted according to the process 1000. However, the computer system may determine that the patient's lower anterior teeth should be adjusted to look straighter and more uniform/aligned with the veneers for the upper anterior teeth. The computer system can then perform the process 1000 for only the lower anterior teeth.

The process 1000 can be performed for one or more both of upper and lower arches. In some implementations, the process 1000 can be performed for any combination of teeth in the upper and lower arches. For example, the process 1000 can be performed for upper anterior teeth (e.g., upper central incisors) and lower posterior teeth (e.g., molars). The process 1000 can be performed for any other combination of teeth in the upper and/or lower arches.

The process 1000 can be performed as part of one or more other processes described herein. For example, the process 1000 can be performed as part of the process 400 in FIGS. 4A and 4B. The computer system can first select at least one set of veneers to achieve the desired teeth arrangement for the patient, and then perform the operations for adjusting the teeth as described in the process 1000.

FIG. 11 is a flowchart of a process 1100 for automatically snapping teeth to an arch form of a digital orthodontics model for auto-smile design. The process 1100 can be used to determine how to move or snap the patient's teeth for aligners, braces, or other types of orthodontic treatment plans, regardless of whether such orthodontic treatment plans include veneers.

The process 1100 can be performed by the digital smile design computer system 102 described in reference to at least FIG. 1A. The process 1100 can also be performed by any other type of computer system, computing device, cloud-based system, and/or network of computing systems. For illustrative purposes, the process 1100 is described from the perspective of a computer system.

Referring to the process 1100 in FIG. 11, the computer system can retrieve or receive a digital orthodontics model with an arch form and patient data for a patient in block 1102. Refer to at least block 1002 in the process 1000 of FIG. 10A.

The computer system can select an upper arch in the digital orthodontics model in block 1104. Although the process 1100 is described as snapping teeth of the upper arch before snapping teeth of a lower arch, the process 1100 can also be performed in which the teeth of the lower arch are snapped to the arch form before the teeth of the upper arch are snapped to the arch form.

In block 1106, the computer system can, for each tooth from a midline to a last molar in the upper arch, snap the tooth tangent to the arch form curve. The computer system can snap central incisors to the midline and tangent to the arch form curve. The computer system can snap lateral teeth. The lateral teeth can be snapped to the arch form similarly to the central incisors. The computer system can snap canines so that cusp tips of the canines are positioned relative the tangent line on the arch form curve and/or a threshold amount outside of the arch form curve. The computer system can, for each molar and upper arch bicuspid tooth, rotate the tooth so that respective marginal ridge datums are tangent to the arch form curve and position the tooth in and out so that the marginal ridge datums are aligned on the arch form curve.

To snap the tooth to the arch form curve, the computer system can identify an existing starting point for the upper arch tooth in block 1108. Refer to block 1008 in the process 1000 of FIG. 10A for further discussion.

The computer system can determine, based on orthodontics rules, a permissible degree of movement to snap the upper arch tooth to the arch form curve in block 1110. The retrieved orthodontics rules and/or the permissible degree of movement can be specific to the particular tooth being moved and/or a particular type of movement being made to the tooth. The permissible degree of movement can further be based on the existing starting point for the tooth and a difference (e.g., offset) between the existing starting point and a desired positioned of the tooth and/or a maximum permissible degree of movement for the tooth. Refer to block 1010 in the process 1000 of FIG. 10A for further discussion.

The computer system can snap the upper arch tooth by the determined permissible degree of movement to be tangent to the arch form curve in block 1112. Snapping the tooth can include moving the tooth in and out, or buccal-lingually. Snapping the tooth can also include rotating the tooth so that a tangent line faces an arch form and/or the tooth achieves a predetermined or desired relationship with the arch form. Snapping the tooth can therefore include moving the tooth relative to the arch form and positioning the tooth on or a certain distance away from the arch form to achieve a predetermined, desired distance from the arch form.

Blocks 1106-1112 can be repeated for each tooth in the upper arch. Blocks 1106-1112 can be repeated for an opposite side of the upper arch. For example, if the blocks 1106-1112 are performed for teeth from the midline to a last molar on a right side of the upper arch, the blocks 1106-1112 can then be repeated for teeth from the midline to a last molar on a left side of the upper arch. Blocks 1106-1112 can be performed for a subset of the teeth in the upper arch. For example, blocks 1106-1112 may be performed only for teeth that are receiving veneers and/or only for teeth that are visible and should be adjusted to improve the patient's appearance and achieve the desired teeth arrangement for the patient.

Sometimes, the process 1100 may stop after adjusting the upper arch. For example, the process 1100 may stop if only the upper arch teeth need to be adjusted to improve the patient's appearance and achieve the desired teeth arrangement. Sometimes, the process 1100 may continue after adjusting the upper arch to block 1114 to then adjust lower arch teeth.

The computer system can select a lower arch in the digital orthodontics model in block 1114.

For each tooth from the midline to a last molar in the lower arch, the computer system can snap the tooth tangent to the arch form curve (block 1116). For example, the computer system can snap lower incisors to a midline and inside the tangent line so that the arch form curve touches a buccal side of the lower incisors. Whereas the computer system may move the upper incisors such that the tangent line is on an inside or lingual side of the upper incisors, the computer system can move the lower incisors such that the tangent line is touching a facial or buccal side of the lower incisors. The computer system can, for each molar and lower posterior tooth, translate the tooth lingually so that a respective buccal cusp tip is positioned on the arch form curve. The lower arch teeth can be rotated similarly to the upper arch teeth rotations described above so that a reference line going through marginal ridge datums in each lower tooth is tangent to the curve.

To snap each tooth, the computer system can identify an existing starting point for the lower arch tooth in block 1118. Block 1118 can be performed similarly to block 1108 in the process 1100.

In block 1120, the computer system can determine, based on the orthodontics rules, a permissible degree of movement to snap the lower arch tooth to the arch form curve. Block 1120 can be performed similarly to block 1110 in the process 1100. As described throughout this disclosure, the permissible degree of movement for the lower arch tooth can be different than the permissible degree of movement for other lower arch teeth and/or upper arch teeth.

The computer system can then snap the lower arch tooth by the determined permissible degree of movement to be tangent to the arch form curve in block 1122. Block 1122 can be performed similarly to block 1112 in the process 100.

Once the teeth are snapped in one or both of the upper and lower arches, the computer system can select a set of veneers based on the patient data in block 1124. Refer to at least block 1032 in the process 1000 of FIG. 10B for further discussion about veneer selection.

The computer system can overlay the snapped teeth in the digital orthodontics model with the selected set of veneers in block 1126. Refer to block 1034 in the process 1000 of FIG. 10B for further discussion.

In block 1128, the computer system can return information indicating at least one of the selected set of veneers, the digital orthodontics model, teeth movement instructions, and/or a dental treatment plan. Refer to at least FIG. 1A and block 1036 in the process 1000 of FIG. 10B for further discussion.

Sometimes, the process 1100 can be performed for the lower arch teeth before the upper arch teeth. Sometimes, the process 1100 can be performed for only one arch of teeth. For example, the computer system may perform the process 1100 only for teeth that may receive veneers. The computer system may perform the process 1100 for teeth receiving the veneers so that the teeth are better aligned to receive the veneers in the desired teeth arrangement for the patient. The computer system may perform the process 1100 only for teeth that may not receive veneers. As an illustrative example, only upper anterior teeth may receive the veneers. The veneers may be fitted to current positioning of the patient's upper anterior teeth. Although these upper anterior teeth may not be adjusted according to the process 1100, the computer system may determine that the patient's lower anterior teeth should be adjusted to look straighter and more uniform/aligned with the veneers for the upper anterior teeth. The computer system can then perform the process 1100 for only the lower anterior teeth.

The process 1100 can be performed as part of one or more other processes described herein. For example, the process 1100 can be performed as part of the process 400 in FIGS. 4A and 4B. The computer system can first select at least one set of veneers to achieve the desired teeth arrangement for the patient, and then perform the operations for adjusting the teeth as described in the process 1100.

FIG. 12 is a flowchart of a process 1200 for automatically resolving IP contacts in a digital orthodontics model for auto-smile design. The process 1200 can be used to determine how to move the patient's teeth for aligners, braces, or other types of orthodontic treatment plans, regardless of whether such orthodontic treatment plans include veneers.

The process 1200 can be performed by the digital smile design computer system 102 described in reference to at least FIG. 1A. The process 1200 can also be performed by any other type of computer system, computing device, cloud-based system, and/or network of computing systems. For illustrative purposes, the process 1200 is described from the perspective of a computer system.

Referring to the process 1200 in FIG. 12, the computer system can retrieve or receive a digital orthodontics model with an arch form and patient data for a patient in block 1202. Refer to at least block 1002 in the process 1000 of FIG. 10A for further discussion.

The computer system can generate a bounding box around each tooth in the digital orthodontics model in block 1204. The computer system can process the model using a machine learning-trained model. The machine learning-trained model can be trained to identify shapes of teeth in the digital orthodontics model and generate a bounding box around each identified shape of a tooth. The computer system can also apply other processing techniques to identify bounding boxes around each tooth in the digital orthodontics model.

In block 1206, for each tooth, the computer system can identify a center point of the tooth as a center point in the respective bounding box. The computer system can determine the center point in the bounding box, then identify that center point as the center of the tooth in the bounding box. In some implementations, the computer system may not perform blocks 1204 and 1206. Instead, each tooth in the digital orthodontics model may already have a coordinate system and identified center point/midpoint.

The computer system can identify vectors between the center points of the teeth in block 1208. The vectors can define directions to move the adjacent teeth in order to achieve a desired IP contact between the adjacent teeth. The vectors for all the teeth can be identified and locked in before adjusting the teeth so that once the computer system moves the teeth along the vectors, a corresponding length of each vector does not change as well. As a result, relative orientation can be maintained between the adjacent teeth while removing any potential interference, overlap, and/or collisions between the adjacent teeth.

The computer system can select a first tooth that is in a defined position (block 1210). The defined position can be near a first side of a midline of the arch form. Thus, the computer system can select the first tooth that is positioned at the midline of the arch. The computer system can then work along one side of the arch starting from the first tooth to a last tooth on the side of the arch. As described further below, once the computer system completes adjusting the teeth on the one side of the arch form, the computer system can select teeth on another side of the arch form that is opposite the first tooth at the midline.

In block 1212, the computer system can identify an nth tooth that is adjacent the first tooth. The nth tooth can be a second tooth that is directly to a right side or a left side of the first tooth.

The computer system can move the first and/or nth tooth along the vector to maintain relative orientation, remove teeth overlap, and/or put the first tooth in contact with the nth tooth at a contact point (block 1214). As part of moving either of the teeth, the computer system can identify existing starting points for each of the first and nth teeth (block 1216). Refer to at least block 1008 in the process 1000 in FIG. 10A for further discussion about identifying the existing starting points.

The computer system can determine, based on orthodontics rules, a permissible degree of movement for each of the first and nth teeth (block 1218). Refer to at least block 1010 in the process 1000 in FIG. 10A for further discussion about determining the permissible degrees of movement per tooth.

The computer system can move the first and/or nth teeth from their existing starting points by the respective determined permissible degrees of movement (block 1220). Refer to at least block 1012 in the process 1000 in FIG. 10A for further discussion about moving the teeth. As described herein, either of the first and nth teeth can be moved along the vector within the permissible degrees of movement. In some implementations, a length of the vector can be determined to correspond to the permissible degrees of movement. In other words, a maximum amount of movement that can be made between the first and nth teeth can be defined as the length of the vector. As a result, the computer system is limited by the length of the vector in how much either of the first and nth teeth are moved relative to each other.

The computer system can then determine in block 1222 whether there are still adjacent teeth to check. For example, the computer system can determine whether another tooth is adjacent the nth tooth, the another tooth being different than the first tooth.

If there are more adjacent teeth to check, the computer system can adjust a vector between the nth tooth and a next adjacent tooth in block 1224. The computer system can then return to block 1214 and iterate through blocks 1214-1220 until all the adjacent teeth have been corrected. Adjusting the vector can include checking the vector length against the orthodontics rules and determining whether the vector length is equal to or within permissible degrees of movement between the nth and next adjacent teeth. If the vector length is not equal or within the permissible degrees of movement, then the computer system can adjust the length to be equal to or within the permissible degrees of movement.

If there are no more adjacent teeth to check in block 1222, the computer system can determine whether there is still another side of the midline that is opposite the defined position for which to resolve IP contacts (block 1226). In other words, if the computer system resolved IP contacts on all teeth right of the midline, the computer system can determine that teeth to a left of the midline should be checked and resolved for IP contacts.

Accordingly, if there is another side of the midline to check, the computer system can select a first tooth near another side of the midline that is opposite the defined position in block 1228. The computer system can then return to block 1212, in which the computer system identifies an nth tooth that is adjacent the first tooth. The computer system can repeat blocks 12-14-1220 until all IP contacts have been resolved on the opposite side of the midline.

If there is not another side of the midline to check in block 1226, the computer system is done checking the teeth to resolve for IP contacts and can select a set of veneers based on the patient data (block 1230). Refer to at least block 1032 in the process 1000 of FIG. 10B for further discussion.

The computer system can overlay the adjusted teeth in the digital orthodontics model with the selected set of veneers in block 1232. Refer to at least block 1034 in the process 1000 of FIG. for further discussion.

The computer system can then return at least the digital orthodontics model in block 1234. Refer to at least block 1036 in the process 1000 of FIG. 10B for further discussion.

In some implementations, the process 1200 can be performed to adjust IP contacts between only some teeth. For example, the computer system may perform the process 1200 only for teeth that may receive veneers. The computer system may perform the process 1200 for teeth receiving the veneers so that the teeth are better aligned and not colliding or otherwise interfering to then receive the veneers in the desired teeth arrangement for the patient. The computer system may perform the process 1200 only for teeth that may not receive veneers. As an illustrative example, only upper anterior teeth may receive the veneers. The veneers may be fitted to current positioning of the patient's upper anterior teeth. The computer system may determine that the patient's lower anterior teeth should be adjusted to look straighter and remove collisions between the lower anterior teeth so that they align better with the veneers. The computer system can then perform the process 1200 for only the lower anterior teeth.

The process 1200 can be performed for one or more both of upper and lower arches. In some implementations, the process 1200 can be performed for any combination of teeth in the upper and lower arches.

The process 1200 can be performed as part of one or more other processes described herein. For example, the process 1200 can be performed as part of the process 400 in FIGS. 4A and 4B. The computer system can first select at least one set of veneers to achieve the desired teeth arrangement for the patient, and then perform the operations for adjusting the teeth as described in the process 1200.

FIG. 13 is a flowchart of a process 1300 for automatically socking teeth in a digital orthodontics model for auto-smile design. The process 1300 can be used to determine how to move the patient's teeth for aligners, braces, or other types of orthodontic treatment plans, regardless of whether such orthodontic treatment plans include veneers. The process 1300 can be performed to sock upper anterior teeth and upper posterior teeth. In some implementations, the process 1300 can first be performed to sock the upper anterior teeth. The process 1300 can then be performed a second time to sock the upper posterior teeth. A similar or same process can also be performed to adjust lower anterior and/or posterior teeth.

The process 1300 can be performed by the digital smile design computer system 102 described in reference to at least FIG. 1A. The process 1300 can also be performed by any other type of computer system, computing device, cloud-based system, and/or network of computing systems. For illustrative purposes, the process 1300 is described from the perspective of a computer system.

Referring to the process 1300 in FIG. 13, the computer system can retrieve a digital orthodontics model with an occlusal plane and patient data for a patient in block 1302. Refer to at least block 1002 in the process 1000 of FIG. 10A for further discussion.

The computer system can select an upper arch of teeth in the digital orthodontics model in block 1304. Although the process 1300 is described in reference to the upper arch, the process 1300 can also be performed for a lower arch.

In block 1306, the computer system can identify a center point between 3 teeth in the selected arch to define a buccal vector as perpendicular to the center point. The buccal vector can be defined for a tooth between 2 of the 3 teeth. The computer system can perform block 1306 for each tooth so that a buccal vector can be defined per tooth. The buccal vectors for the teeth can be defined before proceeding to the next operations in the process 1300, in some implementations. For a last molar, the computer system can use a second-to-last buccal vector that was defined for the upper arch as the last molar's buccal vector. In some implementations, the center point can be defined for a tooth as a midpoint of the tooth. As a result, the computer system may not identify the center point using 3 adjacent teeth.

The computer system can, for each tooth, iteratively adjust the tooth bucally, lingually, down, and/or up based on the buccal vector and respective orthodontics rules (block 1308). In some implementations, the tooth may not be able to be moved up or down, as defined by respective orthodontics rules. Refer to at least blocks 1004-1012 in the process 1000 of FIG. 10A for further discussion about retrieving the orthodontics rules, determining permissible degrees of movement from the retrieved rules, and adjusting the tooth within the permissible degrees of movement.

In block 1310, the computer system can measure a distance (e.g., vertical distance) between the adjusted tooth and a tooth substantially vertically aligned and in an opposite arch. The distance can be taken from tip to tip of the teeth. The distance can be taken from midpoint to midpoint of the teeth. One or more other portions of the teeth may be used for determining the distance.

The computer system can determine whether the distance is within a predetermined threshold distance or whether getting closer to the threshold distance is not possible according to orthodontics rules (block 1312). The predetermined threshold distance can be defined by the orthodontics rules described herein. The predetermined threshold distance can indicate a maximum (or minimum) permissible vertical distance between the teeth. Getting closer to the threshold distance may not be possible if, for example, moving one or more of the teeth any more would cause root damage or other damage to the teeth, gums, and/or jaw. The predetermined threshold distance can vary based on types of the teeth for which the distance is being measured and/or patient data, such as the patient's age, tooth condition, desired teeth arrangement, etc. If the distance is within the predetermined threshold distance, then a permissible amount of movement has been made by the computer system for one or more of the teeth.

If the distance is not within the threshold distance and/or getting closer to the threshold distance is possible, the computer system can reduce the distance in half, in block 1314. In other words, one or more of the teeth can be moved more within the permissible degrees of movement for the teeth. To not overshoot an amount of movement that can be performed again for the teeth, the computer system can reduce the distance of the previous movement in half (e.g., divide by 2). Instead of reducing the distance in half, the computer system can determine a difference between the predetermined threshold distance and the distance that the teeth were previously moved. The computer system can then move the teeth again according to the determined difference.

The computer system can re-adjust the adjusted tooth in an opposite direction of the previous adjustment(s) by the reduced distance amount (block 1316). As described herein, the readjustments can be performed within the permissible degrees of movement or predetermined threshold distance defined by the orthodontics rules.

The computer system can then return to block 1310 and repeat blocks 1310-1316 until the computer system determines that the distance is within the predetermined threshold distance and/or the orthodontics rules do not permit moving the teeth any more to get closer to the threshold distance.

If, in block 1312, the distance is within the predetermined threshold distance and/or getting closer to the threshold distance is not possible according to the orthodontics rules, the computer system can proceed to block 1318, in which the computer system determines whether there is another tooth in the selected arch form to adjust. In other words, the computer system determines that the teeth can no longer be adjusted safely or otherwise according to the orthodontics rules. If the teeth are adjusted anymore, then the adjustments may cause damage to the teeth, roots, gums, and/or jaw of the patient.

If there is another tooth to adjust in the selected arch form, the computer system returns to block 1306 and repeats blocks 1306-1318 until no more teeth need to be adjusted in the selected arch. Sometimes, the patient data can indicate which of the teeth in the selected arch form should be adjusted. The computer system can adjust those teeth as indicated in the patient data. Sometimes, the computer system may only adjust teeth that are in contact with other adjusted teeth. The computer system may adjust teeth that are receiving veneers, in some implementations. Sometimes, the computer system may adjust the teeth that are not receiving veneers so that those teeth fit appropriately with other teeth that are receiving veneers. Other selections of teeth for adjustments are also possible.

If there are no more teeth to adjust in the selected arch, then the computer system can determine whether there is another arch form to select in block 1320. Sometimes, the computer system may only have to adjust teeth in one arch form but not both. Sometimes, the computer system may adjust teeth in both arches. The determination of whether and which arches for which to adjust teeth can depend on a desired teeth arrangement for the patient, positioning of the patient's existing teeth, and the patient data.

If there is another arch form to select, the computer system can select another arch of teeth in the digital orthodontics model (block 1322), then return to block 1306 and iterate through blocks 1306-1320 until there are no other arch forms to select. If there are no other arch forms to select in block 1320, the computer system can proceed to block 1324, in which the computer system selects a set of veneers based on the patient data. Refer to at least block 1032 in the process 1000 of FIG. 10B for further discussion about selecting veneers. In some implementations, if an orthodontics treatment plan is being determined for the patient without installing veneers, then the process 1200 can stop after determining there is no other arch form to select.

The computer system can overlay the adjusted teeth in the digital orthodontics model with the selected set of veneers in block 1326. Refer to at least block 1034 in the process 1000 of FIG. for further discussion.

In block 1328, the computer system can return at least the digital orthodontics model. Refer to at least block 1036 in the process 1000 of FIG. 10B for further discussion.

In some implementations, the process 1300 can be performed to move only some teeth as described herein. The process 1300 can be performed as part of one or more other processes described herein. For example, the process 1300 can be performed as part of the process 400 in FIGS. 4A and 4B. The computer system can first select at least one set of veneers to achieve the desired teeth arrangement for the patient, and then perform the operations for adjusting the teeth as described in the process 1300.

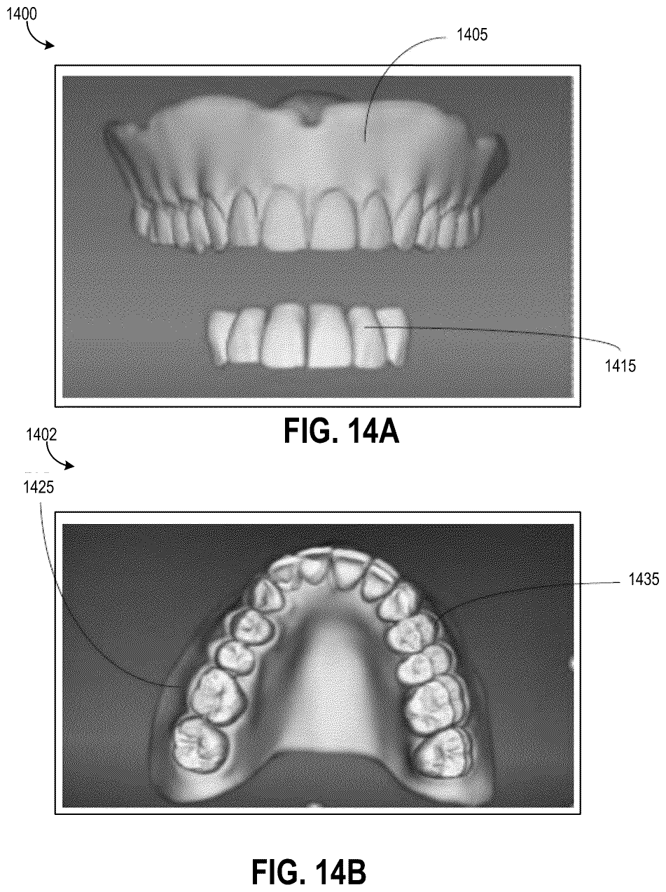

FIGS. 14A and 14B illustrate example GUIs 1400 and 1402, respectively, that may be generated during an automated process of positioning selected veneers in a digital orthodontics model and automatically aligning and leveling each tooth in the model according to an orthodontics treatment plan.

FIG. 14A shows a digital dental model 1405 and a selected set of veneers 1415 (e.g., a selected tooth model). The selected set of veneers 1415 is automatically positioned by the computer system described herein on the digital dental model 1405 to align each tooth with a determined arch and to position each tooth so that it is in a position to achieve a desired teeth arrangement for the patient. In some implementations, an ICP algorithm can be used by the computer system to determine a best fit. The application of the ICP algorithm can be displayed in the GUI 1400 to illustrate to a technician positioning of the teeth and degree of difference that may remain.

In some implementations, an overlay of the set of veneers 1415 and the digital model 1405 from the denture scan can be displayed to the user in the GUI 1400 with a color map or heat map to illustrate differences between the teeth of the set of veneers and the existing scan. In some implementations, the colors of the color map indicate varying degrees of difference between the teeth of the set of veneers and the existing scan. For example, green can indicate that the selected set of veneers and the existing scan are very close, yellow may indicate that the positioning can be improved, and red can indicate that there is a large degree of difference between the selected set of veneers and the existing scan. In some implementations, the colors of the color map can be used to show changes of movement/positioning of the teeth in the model 1405 to achieve the desired teeth arrangement for the patient.

In FIG. 14B, the teeth of the selected set of veneers 1405 are positioned in the digital dental model 1415 in a best-fit position 1425, and differences between the teeth of the digital model 1415 and the teeth of the selected set of veneers 1405 are illustrated to a user by a heat map or color map. This information may be beneficial to identify where and which of the patient's teeth can be adjusted in position to achieve the desired teeth arrangement for the patient. In some implementations, this information can be beneficial to identify whether a different set of veneers should be selected for the patient (e.g., in a scenario where moving the patient's teeth can fall outside permissible degrees of movement defined by orthodontics rules for the particular teeth).

In FIG. 14B, areas where the teeth of the selected set of veneers 1405 extend beyond boundaries of the teeth of the digital dental model 1415 can be colored green (for example position 1435). The technician can use such a display to make adjustments to the digital dental model teeth, so long as those adjustments fall within permissible degrees of movement for the corresponding teeth as defined by the orthodontics rules described herein.

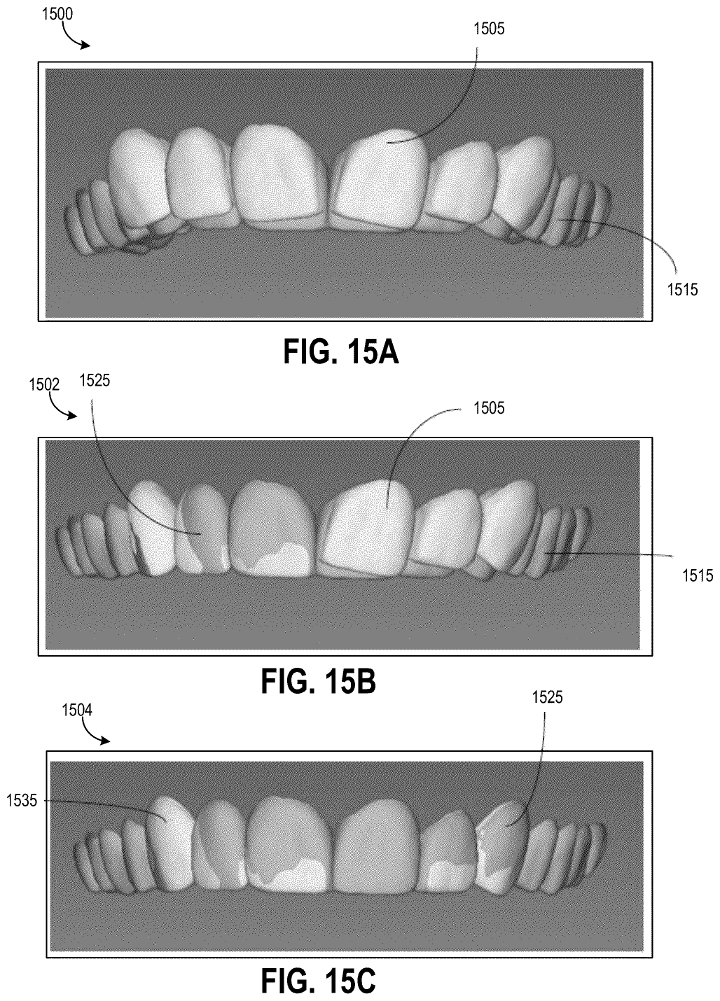

FIGS. 15A, 15B, and 15C illustrate example GUIs 1500, 1502, and 1504, respectively, that may be generated during a process of automatically aligning and leveling each tooth in a digital orthodontics model for auto-smile design.

FIG. 15A shows a selected set of veneers 1505 in an initial position with regard to a digital dental model 1515 in the GUI 1500. Each tooth from the selected set of veneers 1505 is then fit to a corresponding tooth of the digital dental model 1515 by adjusting positions and/or movements of the model 1515 teeth within permissible degrees of movement defined by orthodontics rules. For example, the computer system described herein and/or a user can align one or more of the teeth of the digital model 1515 and/or teeth of the selected set of veneers 1505 with an arch of the digital dental model 1515. As another example, the teeth can be leveled with respect to an occlusal plane.

FIG. 15B shows, in the GUI 1502, a partial completion of the process of FIG. 15A, where teeth on the left side of the digital dental model 1515 have been leveled and aligned to a best-fit position 1525, and teeth of the selected set of veneers 1505 on a right side of the digital model 1515 are not yet fit.

FIG. 15C shows, in the GUI 1504, the completed process of FIG. 15A, where all teeth are in a best-fit position 1525, and areas that the teeth of the selected set of veneers 1505 extend beyond the boundaries of the teeth of the digital model 1515 are colored according to a heat map for presentation to the technician (for example position 1535).

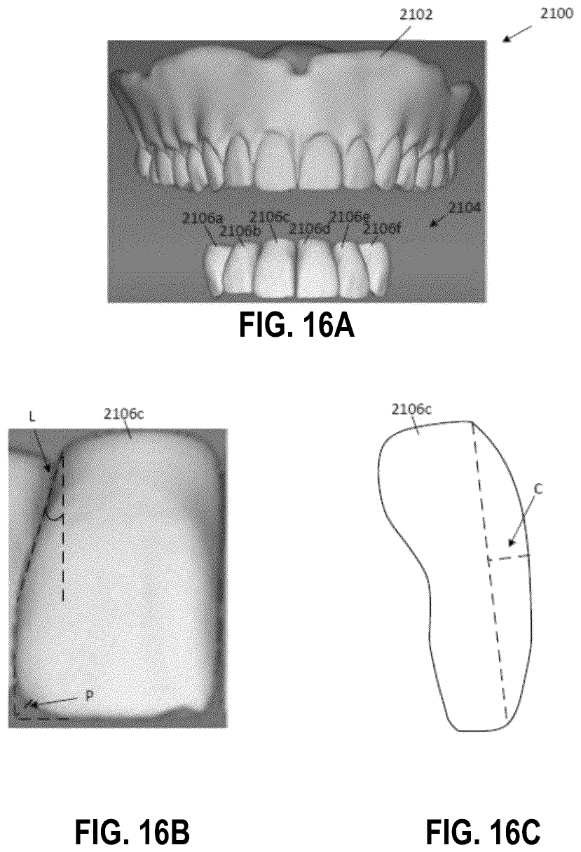

FIGS. 16A, 16B, and 16C illustrate schematic diagrams of an example digital orthodontics model 2102 and example teeth 2106a-f for auto-smile design. FIG. 16A displays a digital reference orthodontics model 2102 along with a portion 2104 of a selected set of veneers. In this example, the portion 2104 includes veneers tooth 2106a, veneers tooth 2106b, veneers tooth 2106c, veneers tooth 2106d, veneers tooth 2106e, and veneers tooth 2106f. The portion 2104 may be displayed for a selected set of veneers (e.g., as selected by the computer system described herein and/or as selected by a relevant user, such as a technician or dentist). A user may review and approve selection of the set of veneers using a user-actuatable control (not shown) displayed on a GUI 2100. In some implementations, the GUI 2100 may include one or more additional user-actuatable controls to load or scroll through different sets of veneers.

The model may be aligned using, for example, an iterative alignment process, such as an iterative closest point (ICP) alignment performed by the computer system described herein. The alignment process may continue for a specific number of iterations or until the transformation calculated/applied by the computer system during an iteration is below a specific threshold. After a first tooth 2106a-n is aligned to the digital model 2102, an adjacent tooth may be positioned next to it by the computer system (e.g., the first tooth 2016a and the adjacent tooth 2106b). After the adjacent tooth is initially positioned next to the aligned tooth of the selected set of veneers, the adjacent tooth may then be aligned with the digital remodel 2102 (e.g., using an alignment technique such as ICP). This process may continue to be performed by the computer system, working from an anterior tooth back to a posterior tooth, one tooth at a time until all of the teeth have been aligned to the digital model 2102.

Teeth from multiple different sets of veneers can be aligned and compared by the computer system. The set of veneers containing the most similar teeth (e.g., based on a calculated similarity values) for achieving a desired teeth arrangement may be selected. In some implementations, teeth from a subset of the different sets of veneers can be used by the computer system. An initial filter (or selection) process may be used to reduce a number of different sets of veneers that are considered. The initial filter process may be based on a width value of one or more anterior teeth, as an illustrative example. The initial filter process may be based on biographic information corresponding to the patient, in yet some implementations.

The initial filter process may also be performed by the computer system and based on extracting a shape from the selected portion of the digital model 2102. For example, multiple horizontal slices of the portion may be generated by the computer system (e.g., by computing the intersection of a horizontal plane with the portion) and compared to each other to determine a general shape of the tooth. This process may be performed by the computer system to determine that the portion of the digital model 2102 has teeth with a square, ovoid, or tapering shape. A subset of sets of veneers may then be identified based on the determined shape. This subset may be aligned and compared to the portion of the digital model 2102 to calculate a similarity value.

The initial filter process may also be based on other properties of the digital model 2102 that are manually or automatically determined. For example, one or more of a point angle, line angle, or labial convexity value may be determined for an anterior tooth portion of the digital model 2102. As shown in FIGS. 16B and 16C, the veneers tooth 2106c is shown with an indicator L of a line angle, an indicator P of a point angle, and indictor C of labial convexity. The indicator L of the line angle shows an angle value that may be determined for the set of veneers by determining the angle of a distal edge of a tooth. The indicator P of the point angle shows a value that corresponds to the roundness of an incisal corner of the tooth. The indicator C shows a value that corresponds to the convexity of the labial surface of the tooth. In some implementations, more than one point angle, line angle, or labial convexity value is determined for the patient's dentition as the tooth may be asymmetric. These and other properties of a patient's teeth may be determined and used to guide the selection of a set of veneers in at least some implementations. Refer to U.S. application Ser. No. 18/328,461, entitled “Auto-Denture Design Setup Systems,” filed on Jun. 2, 2023, the disclosure of which is incorporated by reference in its entirety, for further discussion.

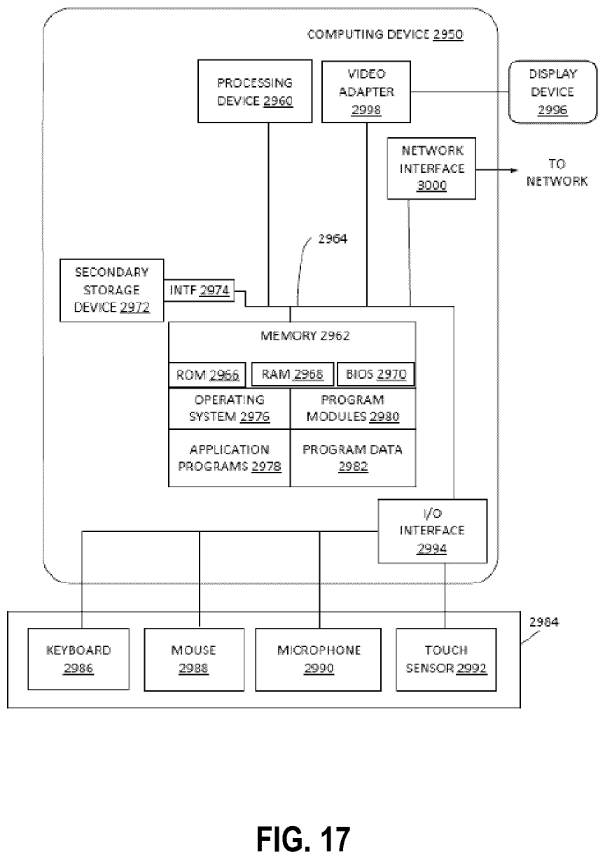

FIG. 17 is an example architecture of a computing device 2950, which can be used to implement aspects according to the present disclosure. The computing device 2950 can be used to execute an operating system, application programs, and software modules to perform the denture design processes described herein. The computing device 2950 includes, in some embodiments, at least one processing device 2960, such as a central processing unit (CPU). Examples of computing devices suitable for the computing device 2950 include a desktop computer, a laptop computer, a tablet computer, a mobile computing device (such as a smartphone, an iPod® or iPad® mobile digital device, or other mobile devices), or other devices configured to process digital instructions. The system memory 2962 includes read only memory 2966 and random-access memory 2968. A basic input/output system 2970 containing the basic routines that act to transfer information within computing device 2950, such as during start up, is typically stored in the read only memory 2966.

The computing device 2950 also includes a secondary storage device 2972 in some embodiments, such as a hard disk drive, for storing digital data. The secondary storage device 2972 is connected to the system bus 2964 by a secondary storage interface 2974. The secondary storage devices 2972 and their associated computer readable media provide nonvolatile storage of computer readable instructions (including application programs and program modules), data structures, and other data for the computing device 2950. A number of program modules can be stored in secondary storage device 2972 or system memory 2962, including an operating system 2976, one or more application programs 2978, other program modules 2980 and program data 2982.

In some embodiments, a user provides inputs to the computing device 2950 through one or more input devices 2984. Examples of input devices 2984 include a keyboard 2986, mouse 2988, microphone 2990, and touch sensor 2992 (such as a touchpad or touch sensitive display). Other embodiments include other input devices 2984. The input devices are often connected to the processing device 2960 through an input/output interface 2994 that is coupled to the system bus 2964. These input devices 2984 can be connected by any number of input/output interfaces, such as a parallel port, serial port, game port, or a universal serial bus. Wireless communication between input devices and the interface 2994 is possible as well, and includes infrared, BLUETOOTH® wireless technology, 802.11a/b/g/n, cellular, ultra-wideband (UWB), ZigBee, or other radio frequency communication systems in some possible embodiments.

In this example embodiment, a display device 2996, such as a monitor, liquid crystal display device, projector, or touch sensitive display device, is also connected to the system bus 2964 via an interface, such as a video adapter 2998. In addition to the display device 2996, the computing device 2950 can include various other peripheral devices (not shown), such as speakers or a printer.

When used in a local area networking environment or a wide area networking environment (such as the Internet), the computing device 2950 is typically connected to the network through a network interface 3000, such as an Ethernet interface or WiFi interface. Other possible embodiments use other communication devices. For example, some embodiments of the computing device 2950 include a modem for communicating across the network.

The computing device 2950 typically includes at least some form of computer readable media. Computer readable media includes any available media that can be accessed by the computing device 2950. By way of example, computer readable media include computer readable storage media and computer readable communication media. Computer readable storage media includes volatile and nonvolatile, removable and non-removable media implemented in any device configured to store information such as computer readable instructions, data structures, program modules or other data. Computer readable storage media includes, but is not limited to, random access memory, read only memory, electrically erasable programmable read only memory, flash memory or other memory technology, compact disc read only memory, digital versatile disks or other optical storage, magnetic cassettes, magnetic tape, magnetic disk storage or other magnetic storage devices, or any other medium that can be used to store the desired information and that can be accessed by the computing device 2950. Computer readable communication media typically embodies computer readable instructions, data structures, program modules or other data in a modulated data signal such as a carrier wave or other transport mechanism and includes any information delivery media. The term “modulated data signal” refers to a signal that has one or more of its characteristics set or changed in such a manner as to encode information in the signal. By way of example, computer readable communication media includes wired media such as a wired network or direct-wired connection, and wireless media such as acoustic, radio frequency, infrared, and other wireless media. Combinations of any of the above are also included within the scope of computer readable media.

The computing device 2950 can also be an example of programmable electronics, which may include one or more such computing devices, and when multiple computing devices are included, such computing devices can be coupled together with a suitable data communication network so as to collectively perform the various functions, methods, or operations disclosed herein.

Although a few implementations have been described in detail above, other modifications are possible. Moreover, other mechanisms for performing the systems and methods described in this document may be used. In addition, the logic flows depicted in the figures do not require the particular order shown, or sequential order, to achieve desirable results. Other steps may be provided, or steps may be eliminated, from the described flows, and other components may be added to, or removed from, the described systems. Accordingly, other implementations are within the scope of the following claims.

What is claimed is:

1. A method for performing an auto-setup of a smile design for a patient using a digital orthodontics model, the method comprising:
accessing, by a computer system, (i) a digital orthodontics model for a patient comprising upper teeth and lower teeth and (ii) an image of the patient, wherein the image comprises at least the patient's existing teeth;
accessing, by the computer system and from a data store, orthodontics rules comprising permissible degrees of movement for each tooth;
determining, by the computer system and based on the orthodontics rules, tooth movements along an arch form defined in the digital orthodontics model to achieve a desired teeth arrangement for the patient, wherein the tooth movements are made within the permissible degrees of movement for each tooth;
selecting, by the computer system and from the data store, a set of veneers from amongst a plurality of sets of veneers to achieve the desired teeth arrangement for the patient, the set of veneers being selected based at least in part on the determined tooth movements;
overlaying, by the computer system, the digital orthodontics model with teeth of the selected set of veneers;
augmenting, by the computer system, the image of the patient's existing teeth with the digital orthodontics model having the teeth of the selected set of veneers, wherein the augmented image comprises a realistic visual depiction of the patient with the teeth of the selected set of veneers; and
returning, by the computer system, at least the augmented image.

2. The method of claim 1, further comprising generating, by the computer system, tooth movement instructions based on the determined tooth movements and the digital orthodontics model having the teeth of the selected set of veneers.

3. The method of claim 1, further comprising generating, by the computer system, an orthodontics treatment plan based on the determined tooth movements and the digital orthodontics model having the teeth of the selected set of veneers.

4. The method of claim 1, further comprising generating and returning veneers information based on the digital orthodontics model having the teeth of the selected set of veneers, the veneers information including a type of the selected set of veneers and placement of the selected set of veneers in the patient's mouth.

5. The method of claim 1, further comprising iteratively adjusting, by the computer system, positions of one or more teeth in the digital orthodontics model based on the orthodontics rules and until the desired teeth arrangement for the patient is achieved by the iterative adjustments.

6. The method of claim 1, wherein determining, by the computer system and based on the orthodontics rules, tooth movements along an arch form defined in the digital orthodontics model to achieve a desired teeth arrangement for the patient comprises:
leveling each tooth relative to an occlusal plane within respective permissible degrees of leveling movement defined by the orthodontics rules for the tooth.

7. The method of claim 1, wherein determining, by the computer system and based on the orthodontics rules, tooth movements along an arch form defined in the digital orthodontics model to achieve a desired teeth arrangement for the patient comprises:
rotating at least one tooth around a reference line perpendicular through marginal ridges of the at least one tooth, wherein the rotating is within permissible degrees of rotating movement defined by the orthodontics rules for the at least one tooth.

8. The method of claim 1, wherein determining, by the computer system and based on the orthodontics rules, tooth movements along an arch form defined in the digital orthodontics model to achieve a desired teeth arrangement for the patient comprises:
torqueing at least one tooth using at least one of buccal cusp tips and distal cusp tips of the at least one tooth, wherein the torqueing is within permissible degrees of torqueing movement defined by the orthodontics rules for the at least one tooth.

9. The method of claim 1, wherein determining, by the computer system and based on the orthodontics rules, tooth movements along an arch form defined in the digital orthodontics model to achieve a desired teeth arrangement for the patient comprises:
tipping at least one tooth at least one of mesially and distally using marginal ridges of the at least one tooth, wherein the tipping is within permissible degrees of tipping movement defined by the orthodontics rules for the at least one tooth.

10. The method of claim 1, wherein determining, by the computer system and based on the orthodontics rules, tooth movements along an arch form defined in the digital orthodontics model to achieve a desired teeth arrangement for the patient comprises:
snapping at least one tooth to the arch form, wherein the snapping is within permissible degrees of snapping movement defined by the orthodontics rules for the at least one tooth.

11. The method of claim 1, wherein determining, by the computer system and based on the orthodontics rules, tooth movements along an arch form defined in the digital orthodontics model to achieve a desired teeth arrangement for the patient comprises:
adjusting interproximal (IP) contacts between adjacent teeth, wherein the adjusting is within permissible degrees of IP-contact movement defined by the orthodontics rules for the at least one tooth.

12. The method of claim 11, wherein adjusting IP contacts comprises:
for each tooth of the adjacent teeth, generating a bounding box;
identifying a center point of the tooth as a center point in the bounding box;
identifying a vector between center points of the adjacent teeth, wherein a length of the vector is equal to or within respective permissible degrees of IP-contact movement for the adjacent teeth; and
moving at least one tooth of the adjacent teeth along the vector and within the respective permissible degrees of IP-contact movement for the adjacent teeth to maintain relative orientation, remove overlap, and put the adjacent teeth in contact at a predefined contact point.

13. The method of claim 1, wherein selecting, by the computer system and from the data store, a set of veneers from amongst a plurality of sets of veneers to achieve the desired teeth arrangement for the patient comprises:
identifying a shape of the patient's existing teeth in the digital orthodontics model;
scoring each set of the plurality of sets of veneers based on at least one of (i) a likelihood that the set achieves the desired teeth arrangement and (ii) the set has a shape similar to the shape of the patient's existing teeth; and
selecting the set of veneers from the plurality of sets of veneers having a highest score.

14. The method of claim 13, wherein selecting the set of veneers comprises selecting a subset of the plurality of sets of veneers, wherein each set of veneers in the subset has a score that exceeds a threshold score value.

15. The method of claim 1, wherein determining, by the computer system and based on the orthodontics rules, tooth movements along an arch form defined in the digital orthodontics model to achieve a desired teeth arrangement for the patient comprises, for each tooth:
determining a starting point of the tooth;
identifying, based on the orthodontics rules, a maximum permissible degree of movement for the tooth;
determining an offset between the starting point of the tooth and the maximum permissible degree of movement for the tooth; and
moving the tooth according to the determined offset.

16. The method of claim 1, further comprising transmitting the augmented image to a user device for presentation in a graphical user interface (GUI) at the user device.

17. The method of claim 1, further comprising:
transmitting, by the computer system, the digital orthodontics model with the teeth of the selected set of veneers to a user device for presentation in a GUI at the user device;
receiving, by the computer system and from the user device, user input indicating approval of the selected set of veneers; and
generating, by the computer system and based on receiving the user input, an orthodontics treatment plan for (i) adjusting the patient's existing teeth according to the determined tooth movements and (ii) applying the teeth of the selected set of veneers to the patient's existing teeth.

18. The method of claim 16, further comprising:
receiving, by the computer system and from the user device, user input indicating a request for other sets of veneers from the plurality of sets of veneers, wherein the other sets of veneers do not include the selected set of veneers;
selecting, by the computer system and from amongst the plurality of sets of veneers, the other sets of veneers; and
transmitting, by the computer system to the user device, the other sets of veneers for presentation in the GUI at the user device, wherein the presented other sets of veneers are user-selectable.

19. A method for performing an auto-setup of a smile design for a patient using a digital orthodontics model, the method comprising:
accessing, by a computer system, patient data comprising (i) a digital orthodontics model for a patient, the digital orthodontics model including upper teeth and lower teeth and (ii) an image of the patient, wherein the image comprises at least the patient's existing teeth;
selecting, by the computer system and from a data store, a set of veneers from amongst a plurality of sets of veneers to achieve a desired teeth arrangement for the patient, the set of veneers being selected based at least in part on the patient data;
overlaying, by the computer system, the digital orthodontics model with teeth of the selected set of veneers;
retrieving, by the computer system and from the data store, orthodontics rules comprising permissible degrees of movement for each tooth;
determining, by the computer system and based on the orthodontics rules, tooth movements along an arch form defined in the digital orthodontics model to achieve the desired teeth arrangement for the patient of the digital orthodontics model with the teeth of the selected set of veneers, wherein the tooth movements are made within the permissible degrees of movement for each tooth;
for each tooth movement, determining, by the computer system, whether the tooth movement satisfies corresponding permissible degrees of movement for the tooth;
generating, by the computer system and based on a determination that the tooth movement satisfies the corresponding permissible degrees of movement for the tooth, tooth movement data;
augmenting, by the computer system, the image of the patient's existing teeth with the digital orthodontics model having the teeth of the selected set of veneers and the determined tooth movements, wherein the augmented image comprises a realistic visual depiction of the patient with the teeth of the selected set of veneers; and
returning, by the computer system, at least the tooth movement data and the augmented image.

20. The method of claim 19, further comprising:
generating, by the computer system and based on the tooth movement data, an orthodontics treatment plan; and
returning, by the computer system, the orthodontics treatment plan for adjusting the patient's existing teeth according to the determined tooth movements.
